(19) United States
(12) Patent Application Publication
(10) Pub. No.: US 2007/0269375 A1
(43) Pub. Date: Nov. 22, 2007

(54) Stable Radiopharmaceutical Compositions and Methods for Preparation

(75) Inventors: Jianqing Chen, Bordentown, NJ (US); Karen E. Linder, Kingston, NJ (US); Edmund R. Marinelli, Lawrenceville, NJ (US); Edmund Metcalfe, Kingston, NJ (US); Adrian D. Nunn, Lamberville, NJ (US); Rolf E. Swenson, Princeton, NJ (US); Michael F. Tweedle, Princeton, NJ (US)

Correspondence Address: KRAMER LEVIN NAFTALIS & FRANKEL LLP, INTELLECTUAL PROPERTY DEPARTMENT, 1177 AVENUE OF THE AMERICAS, NEW YORK, NY, 10036, US

(73) Assignee: Bracco Imaging S.p.A., Milan (IT)

(21) Appl. No.: 10/566,112

(22) PCT Filed: Jul. 23, 2004
(86) PCT No.: PCT/US04/23930

§ 371 Date: Jul. 9, 2007

Related U.S. Application Data
(60) Provisional application No. 60/489,850, filed on Jul. 24, 2003.

(51) Int. Cl. A61K 51/08; A61K 103/10; A61K 103/30; A61K 103/36; A61K 103/40; A61K 31/198

(52) U.S. Cl. 424/1.69; 424/1.65; 514/561

(57) ABSTRACT

Stabilized radiopharmaceutical formulations are disclosed. Methods of making and using stabilized radiopharmaceutical formulations are also disclosed. The invention relates to stabilizers that improve the radiostability of radiotherapeutic and radiodiagnostic compounds, and formulations containing them. In particular, it relates to stabilizers useful in the preparation and stabilization of targeted radiodiagnostic and radiotherapeutic compounds, and, in a preferred embodiment, to the preparation and stabilization of radiodiagnostic and radiotherapeutic compounds that are targeted to the Gastrin Releasing Peptide Receptor (GRP-Receptor).

### CROSS REFERENCE TO RELATED APPLICATIONS

This application claims benefit of U.S. Provisional Application No. 60/489,850 filed Jul. 24,2003, which is hereby incorporated by reference in its entirety.

### FIELD OF THE INVENTION

This invention relates to stabilizers that improve the radiostability of radiotherapeutic and radiodiagnostic compounds, and formulations containing them. In particular, it relates to stabilizers useful in the preparation and stabilization of targeted radiodiagnostic and radiotherapautic compounds, and, in a preferred embodiment, to the preparation and stabilization of radiodiagnostic and radiotherapeutic compounds that are targeted to the Gastin Releasing Peptide Receptor (GRP-Receptor).

### BACKGROUND OF THE INVENTION

Radiolabeled compounds designed for use as radiodiagnostic agents are generally prepared with a gamma-emitting isotope as the radiolabel. These gamma photons penetrate water and body tissues readily and can have a range in tissue or air of many centimeters, In general, such radiodiagnostic compounds do not cause significant damage to the organ systems that are imaged using these agents. This is because the gamma photons given off have no mass or charge and the amount of radioactive material that is injected is limited to the quantity required to obtain a diagnostic image, generally in the range of about 3 to 50 mCi, depending on the isotope and imaging agent used. This quantity is small enough to obtain useful images without significant radiation dose to the patient. Radionuclides such as ^{99m}Tc, ¹¹¹In ¹²³I, ⁵⁷Ga and ⁶⁴Cu have been used for this purpose.

In contrast, radiolabeled compounds designed for use as radiotherapeutic agents are generally labeled with an Auger-, beta- or an alpha-emitting isotope, which may optionally also give off gamma photons. Radionuclides such as ⁹⁰Y, ¹⁷⁷Lu, ¹⁴⁹Pm, ¹⁵³Sm, ¹⁰⁹Pd, ⁶⁷Cu, ¹⁶⁶Ho, ¹³¹I, ³²P, ^{186/188}Re, ¹⁰⁵Rh, ²¹¹At, ²²⁵Ac, ⁴⁷Sc, ²¹³Bi, and others, are potentially useful for radiotherapy. The +3 metal ions of the lanthanide isotopes are of particular interest, and include ¹⁷⁷Lu (relatively low energy β-emitter), ¹⁴⁹Pm, ¹⁵³Sm (medium energy) and ¹⁶⁶Ho (high energy). ⁹⁰Y also forms a +3 metal ion, and has coordination chemistry that is similar to that of the lanthanides. The coordination chemistry of the lanthanides is well developed and well known to those skilled in the art.

The ionizing radiation given off from compounds labeled with these radioisotopes is of an appropriate energy to damage cells and tissue in sites where the radiolabelled compound has localized. The radiation emitted can either damage cellular components in the target tissue directly, or can cause water in tissues to form free radicals. These radicals are very reactive and can damage proteins and DNA.

Some of the immediate products that form from the radiolysis of water are outlined below.

H₂O→H₂O⁺+e⁻

H₂O⁺→H⁺+OH*

H2O+e⁻→H₂O⁻→H*+OH⁻

Of the products that form, (e.g. H⁺, OH⁻, H*, and OH*), the hydroxyl radical [OH*] is particularly destructive. This radical can also combine with itself to form hydrogen peroxide, which is a strong oxidizer.

OH*+OH*→H₂O₂ (strong oxidizer)

In addition, interaction of ionizing radiation with dissolved oxygen can generate very reactive species such as superoxide radicals. These radicals are very reactive towards organic molecules (see, e.g. Garrison, W. M., Chem. Rev. 1987, 87, 381-398).

Production of such reactive species at the site or sites that the radiotherapeutic or radiodiagnostic compound is targeted to (e.g., a tumor, bone metastasis, blood cells or other targeted organ or organ system) will, if produced in sufficient quantity, have a cytostatic or cytotoxic effect. The key factor for successful radiotherapy is the delivery of enough radiation dose to the targeted tissue (e.g., tumor cells, etc.) to cause a cytotoxic or tumoricidal effect, without causing significant or intolerable side effects. Similarly, for a radiodiagnostic, the key factor is delivery of sufficient radiation to the target tissue to image it without causing significant or intolerable side effects.

Alpha particles dissipate a large amount of energy within one or two cell diameters, as their range of penetration in tissues is only ˜50 μm. This can cause intense local damage, especially if the radiolabeled compound has been internalized into the nucleus of the cell. Likewise, radiotherapeutic compounds labeled with Auger-electron emitters such as ¹¹¹In have a very short range and can have potent biological- effects at the desired site of action. The emissions from therapeutic beta-emitting isotopes such as ¹⁷⁷Lu or ⁹⁰Y have somewhat longer ranges in tissue, but again, most of the damage produced occurs within a few millimeters or centimeters from the site of localization.

However, the potentially destructive properties of the emissions of a radiotherapeutic isotope are not limited to their cellular targets. For radiotherapeutic and radiodiagnostic compounds, radiolytic damage to the radiolabeled compound itself can be a serious problem during the preparation, purification, storage and/or shipping of a radiolabeled radiotherapeutic or radiodiagnostic compound, prior to its intended use.

Such radiolytic damage can cause, for example, release of the radioisotope [e.g., by dehalogenation of radioiodinated antibodies or decomposition of the chelating moiety designed to hold the radiometal], or it can damage the targeting molecule that is required to deliver the targeted agent to its intended target. Both types of damage are highly undesirable as they can potentially cause the release of unbound isotope, e.g., free radioiodine or unchelated radiometal to the thyroid, bone and other organs, or cause a decrease or abolishment of targeting ability as a result of radiolytic damage to the targeting molecule, such as a receptor-binding region of a targeting peptide or radiolabeled antibody. Radioactivity that does not become associated with its target tissue may be responsible for unwanted side effects.

For example, DOTA-Gly-ACA-Gln-Trp-Ala-Val-Gly-His-Leu-Met-NH₂ (ACA=3-Amino-3-deoxycholic acid) and DOTA-Gly-Abz4-Gln-Trp-Ala-Val-Gly-His-Leu-Met-NH₂ (Abz4=4-aminobenzoic acid) the two chelating ligands shown in FIGS. 1 and 2, respectively, have been shown to specifically target the Gastrin Releasing Peptide (GRP) Receptors. In the examples that follow, these have been described as Compounds A and Compound B respectively. Other GRP receptor-binding ligands are described in U.S. Pat. No. 6,200,546, to Hoffmnan et al., published U.S. application U.S. 2002/0054855, and in copending application Ser. No. 10/341,577, filed Jan. 13, 2003, the entire contents of which are incorporated by reference.

When radiolabeled with diagnostic and radiotherapeutic radionuclides such as ¹¹¹In and ¹⁷⁷Lu, Compounds A and B have been shown to have high affinity for GRP receptors, both in vitro and in vivo. However, these compounds can undergo significant radiolytic damage that is induced by the radioactive label if these radiolabeled complexes are prepared without concomitant or subsequent addition of one or more radiostabilizers (compounds that protect against radiolytic damage). This result is not surprising, as the hydroxyl and superoxide radicals generated by the interaction of β-particles with water are highly oxidizing. Radiolytic damage to the methionine (Met) residue in these peptides is the most facile mode of decomposition, possibly resulting in a methionine sulfoxide derivative.

Cell binding results show that the resulting radiolytically damaged derivatives are devoid of GRP-receptor binding activity (IC₅₀ values greater than micromolar). Hence, it is critical to find inhibitors of radiolysis that can be used to prevent both methionine oxidation and other radiolytic decomposition routes in radiodiagnostic and radiotherapeutic compounds.

Preventing such radiolytic damage is a major challenge in the formulation of radiodiagnostic and radiotherapeutic compounds. For this purpose, compounds known as radical scavengers or antioxidants are typically used. These are compounds that react rapidly with, e.g., hydroxyl radicals and superoxide, thus preventing them from reaction with the radiopharmaceutical of interest or reagents for its preparation.

There has been extensive research in this area. Most of it has focused on the prevention of radiolytic damage in radiodiagnostic formulations, and several radical scavengers have been proposed for such use. However, it has been found in the studies described herein that the stabilizers reported to be effective by others, provide insufficient radiostabilization to protect ¹⁷⁷Lu-A and ¹⁷⁷Lu-B, the Lutetium complexes of Compounds A and B, respectively, from radiolytic damage, especially when high concentrations and large amounts of radioactivity are used.

For example, Cyr and Pearson [Stabilization of radiopharmaceutical compositions using hydrophilic thioethers and hydrophilic 6-hydroxy chromans. Cyr, John E.; Pearson, Daniel A. (Diatide, Inc., USA). PCT Int. Appl. (2002), WO 200260491 A2 20020808] state that diagnostic and therapeutic radiopharmaceutical compositions radiolabeled with ¹²⁵I, ¹³¹I, ²¹¹At, ⁴⁷Sc, ⁶⁷Cu, ⁷²Ga, ⁹⁰Y, ¹⁵³Sm, ¹⁵⁹Gd, ¹⁶⁵Dy, ¹⁶⁶Ho, ¹⁷⁵Yb, ¹⁷⁷Lu, ²¹²Bi, ²¹³Bi, ⁶⁸Ga, ^{99m}Tc, ¹¹¹In and ¹²³I can be stabilized by the addition of a hydrophilic thioether, and that the amino acid methionine, a hydrophilic thioether, is especially useful for this purpose.

A study was therefore performed wherein L-methionine (5 mg/mL) was added to ¹⁷⁷Lu-A, to evaluate its ability to serve as a radical scavenger. As will be described in more detail below, reverse phase HPLC shows that after five days, almost complete decomposition of ¹⁷⁷Lu-A had occurred, indicating that the radiostabilizer used was insufficient to prevent radiolytic damage. In vitro binding results indicate that such decomposition can dramatically decrease the potency and targeting ability, and hence the radiotherapeutic efficacy, of the compound thus damaged. To attain the desired radiotherapeutic effect, one would need to inject more radioactivity, thus increasing the potential for toxicity to normal organs.

In order to identify suitable antioxidant radical scavengers that might be useful for the radiostabilization of GRP-receptor binding radiodiagnostic and radiotherapeutic compounds, several studies were performed. One or more potential radiostabilizers was added after complex formation (a two-vial formulation) or they were added directly to the reaction mixture prior to complexation with a radiometal (or both). Ideally, the radiostabilizer should be able to be added directly to the formulation without significantly decreasing the radiochemical purity (RCP) of the product, as such a formulation has the potential to be a single-vial kit.

The radical scavengers identified as a result of these studies have general utility in formulations for the preparation of compounds used for a variety of radiodiagnostic and radiotherapeutic applications, and may be useful to stabilize compounds radiolabeled with a variety of isotopes, e.g., ^{99m}Tc, ^{186/188}Re, ¹¹¹In, ⁹⁰y, ¹⁷⁷Lu, ²¹³Bi, ²²⁵Ac, ¹⁶⁶Ho and others. The primary focus of the examples in this application is the radiostabilization of GRP-binding peptides, and in particular, the radioprotection of methionine residues in these molecules. However, the stabilizers identified should have applicability to a wide range of radiolabeled peptides, peptoids, small molecules, proteins, antibodies, and antibody fragments and the like. They are useful for the radioprotection of any compound that has a residue or residues that are particularly sensitive to radiolytic damage, such as, for example, tryptophan (oxidation of the indole ring), tyrosine (oxidative dimerization, or other oxidation), histidine, cysteine (oxidation of thiol group) and to a lesser extent serine, threonine, glutamic acid, and aspartic acid. Unusual amino acids commonly used in peptides or drugs that contain sensitive functional groups (indoles, imidazoles, thiazoles, furans, thiophenes and other heterocycles) could also be protected.

### SUMMARY OF THE INVENTION

It is the aim of this invention to provide stabilizers and stabilizer combinations that slow or prevent radiolytic damage to targeted radiotherapeutic and radiodiagnostic radiolabeled compounds, especially compounds labeled with radiometals, and thus preserve the targeting ability and specificity of the compounds. It is also an aim to present formulations containing these stabilizers. As described by the examples below, many stabilizers have been identified that, alone or in combination, inhibit radiolytic damage to radiolabeled compounds. At this time, four approaches are particularly preferred. In the first approach, a radiolysis stabilizing solution containing a mixture of the following ingredients is added to the radiolabeled compound immediately following the radiolabeling reaction: gentisic acid, ascorbic acid, human serum albumin, benzyl alcohol, a physiologically acceptable buffer or salt solution at a pH of about 4.5 to about 8.5, and one or more amino acids selected from methionine, selenomethionine, selenocysteine, or cysteine).

The physiologically acceptable buffer or salt solution is preferably selected from phosphate, citrate, or acetate buffers or physiologically acceptable sodium chloride solutions or a mixture thereof, at a molarity of from about 0.02M to about 0.2M. The reagent benzyl alcohol is a key component in this formulation and serves two purposes. For compounds that have limited solubility, one of its purposes is to solubilize the radiodiagnostic or radiotherapeutic targeted compound in the reaction solution, without the need for added organic solvents. Its second purpose is to provide a bacteriostatic effect. This is important, as solutions that contain the radiostabilizers of the invention are expected to have long post-reconstitution stability, so the presence of a bacteriostat is critical in order to maintain sterility. The amino acids methionine, selenomethionine, cysteine, and selenocysteine play a special role in preventing radiolytic damage to methionyl residues in targeted molecules that are stabilized with this radiostabilizing combination.

In the second approach, stabilization is achieved via the use of dithiocarbamate compounds having the following general formula: 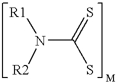
wherein R1 and R2 are each independently H, C1-C8 alkyl, —OR3, wherein R3 is C1-C8 alkyl, or benzyl (Bn) (either unsubstituted or optionally substituted with water solubilizing groups),
or wherein R1R2N combined=1-pyrrolidinyl-, piperidino-, morpholino-, 1-piperazinyl- and M=H⁺, Na⁺, K⁺, NH₄⁺, M-methylglucamine, or other pharmaceutically acceptable +1 ions.

Alternatively, compounds of the form shown below may be used, wherein M is a physiologically acceptable metal in the +2 oxidation state, such as Mg²⁺ or Ca²⁺, and R1 and R2 have the same definition as described above.

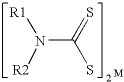

These reagents can either be added directly into reaction mixtures during radiolabeled complex preparation, or added after complexation is complete, or both.

The compound 1-Pyrrolidine Dithiocarbamic Acid Ammonium salt (PDTC) proved most efficacious as a stabilizer, when either added directly to the reaction mixture or added after complex formation. These results were unexpected, as the compound has not been reported for use as a stabilizer for radiopharmaceuticals prior to these studies. Dithiocarbamates, and PDTC in particular, have the added advantage of serving to scavenge adventitious trace metals in the reaction mixture.

In the third approach, formulations contain stabilizers that are water soluble organic selenium compounds wherein the selenium is in the oxidation state +2. Especially preferred are the amino acid compounds selenomethionine, and selenocysteine and their esters and amide derivatives and dipeptides and tri peptides thereof, which can either be added directly to the reaction mixture during radiolabeled complex preparation, or following complex preparation. The flexibility of having these stabilizers in the vial at the time of labeling or in a separate vial extends the utility of this invention for manufacturing radiodiagnostic or radiotherapeutic kits.

It is highly efficacious to use these selenium compounds in combination with sodium ascorbate or other pharmaceutically acceptable forms of ascorbic acid and its derivatives.

The ascorbate is most preferably added after complexation is complete. Alternatively, it can be used as a component of the stabilizing formulation described above. A fourth approach involves the use of water soluble sulfur-containing compounds wherein the sulfur in the +2 oxidation state. Preferred thiol compounds include derivatives of cysteine, mercaptothanol, and dithiolthreotol. These reagents are particularly preferred due to their ability to reduce oxidized forms of methionine residues (e.g., methionine oxide residues) back to methionyl residues, thus restoring oxidative damage that has occurred as a result of radiolysis. With these thiol compounds, it is highly efficacious to use these stabilizing reagents in combination with sodium ascorbate or other pharmaceutically acceptable forms of ascorbic acid and its derivatives. The ascorbate is most preferably added after complexation is complete.

The stabilizers and stabilizer combinations may be used to improve the radiolytic stability of targeted radiopharmaceuticals, comprising peptides, non-peptidic small molecules, radiolabeled proteins, radiolabeled antibodies and fragments thereof. These stabilizers are particularly useful with the class of GRP-binding compounds described herein.

### BRIEF DESCRIPTION OF THE DRAWINGS

FIG. 1 shows the structure of Compound A.

FIG. 2 shows the structure of Compound B.

FIG. 3 illustrates the results of an HPLC analysis of a mixture of ¹⁷⁷Lu-A with 2.5 mg/mL L-Methionine over 5 days at room temperature at a radioconcentration of 25 mCi/mL. [50 mCi total]. FIG. 3A is a radiochromatogram of a reaction mixture for the preparation of ¹⁷⁷Lu-A, which was initially formed in >98% yield. FIG. 3B is radiochromatogram of [¹⁷⁷Lu-A], 25 mCi/mL, after five days at room temperature, demonstrating complete radiolytic destruction of the desired compound. The radiostabilizer added (5 mg/mL L-Methionine) was clearly insufficient for the level of radioprotection required.

FIG. 4 is an HPLC trace [radiodetection] showing that ¹⁷⁷Lu-B (104 mCi) has >99% RCP for 5 days when diluted 1:1 with radiolysis protecting solution that was added after the complex was formed.

FIG. 5 is an HPLC trace [radiodetection] showing that ¹⁷⁷Lu-A has >95% RCP for 5 days at a concentration of 55 mCi/2 mL if 1 mL of radiolysis protecting solution is added after the complex was formed.

FIG. 6A and FIG. 6B show the structure of the methionine sulfoxide derivative of ¹⁷⁷Lu-A (FIG. 6A) and methionine sulfoxide derivative of ¹¹¹In-B (FIG. 6B).

FIG. 7A and FIG. 7B show stabilizer studies ¹⁷⁷Lu-A (FIG. 7A) and ¹⁷⁷Lu-B (FIG. 7B). Radioactivity traces are shown from a study to compare the radiostabilizing effect of different amino acids, when added to ¹⁷⁷Lu-A (FIG. 7A) and ¹⁷⁷Lu-B (FIG. 7B) at an amino acid concentration of 6.6 mg/mL in 10 mM Dulbecco's phosphate buffered saline, pH 7.0 [PBS], and a radioactivity concentration of 20 mCi/mL, after 48 hours of storage at room temperature. A total of 3.5 mCi of ¹⁷⁷Lu was added to each vial. A full description of the experimental procedure is given in Example 1.

FIG. 8 shows an HPLC trace [radiodetection] showing the radiostability of ¹⁷⁷Lu-A over 5 days at room temperature at a radioconcentration of 25 mCi/mL in presence of 2.5 mg/mL L-methionine (50 mCi total). The details of this study are given in Example 2.

FIG. 9 shows an HPLC trace [radiodetection] showing the stability of ¹⁷⁷Lu-B at a concentration of 50 mCi/2 mL in a radiolysis protecting solution containing L-methionine. The details of this study are given in Example 4.

FIGS. 10A-C show radiochromatograms and UV chromatograms comparing samples with and without 1-pyrrolidine dithiocarbamic acid ammonium salt in the reaction buffer and containing zinc as a contaminant metal during the reaction of ¹⁷⁷Lu-B. The experimental procedure for this study is given in Example 20.

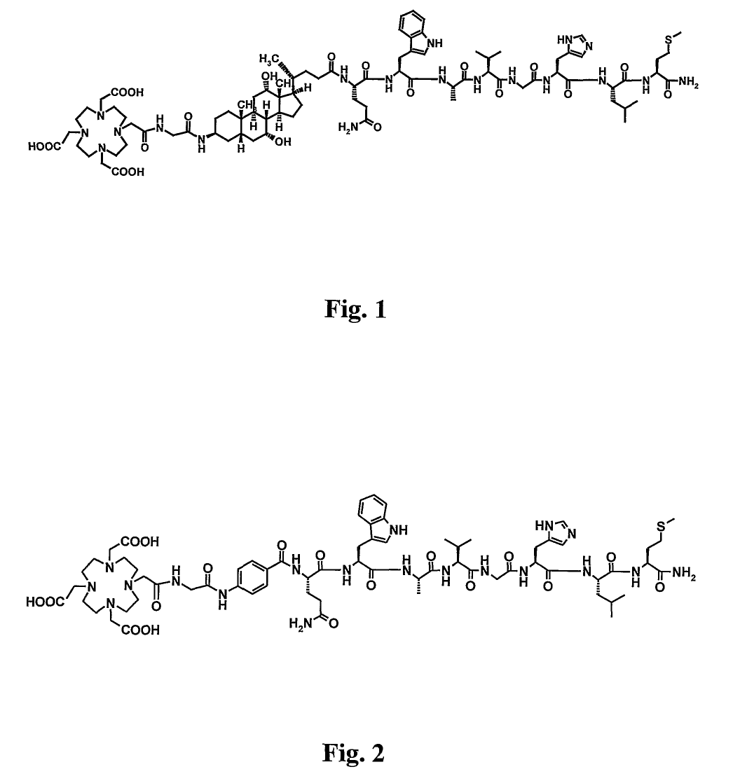

### DETAILED DESCRIPTION OF THE INVENTION

In the following description, various aspects of the present invention will be further elaborated. For purposes of explanation, specific configurations and details are set forth in order to provide a thorough understanding of the present invention. However, it will also be apparent to one skilled in the art that the present invention may be practiced without the specific details.

Furthermore, well known features may be omitted or simplified in order not to obscure the present invention.

### 1. Metal Chelator

In some radiopharmaceuticals, the isotope is a non-metal, such as ¹²³I, ¹³¹I or ¹⁸F, and is either coupled directly to the rest of the molecule or is bound to a linker. However, if the radioisotope used is a metal, it is generally incorporated into a metal chelator. The term “metal chelator” refers to a molecule that forms a complex with a metal atom. For radiodiagnostic and radiotherapeutic applications it is generally preferred that said complex is stable under physiological conditions. That is, the metal will remain complexed to the chelator backbone in vivo. In a preferred embodiment, a metal chelator is a molecule that complexes to a radionuclide metal to form a metal complex that is stable under physiological conditions and which also has at least one reactive functional group for conjugation with a targeting molecule, a spacer, or a linking group, as defined below. The metal chelator M may be any of the metal chelators known in the art for complexing a medically useful metal ion or radionuclide. The metal chelator may or may not be complexed with a metal radionuclide. Furthermore, the metal chelator can include an optional spacer such as a single amino acid (e.g., Gly) which does not complex with the metal, but which creates a physical separation between the metal chelator and the linker.

The metal chelators of the invention may include, for example, linear, macrocyclic, terpyridine, and N₃S, N₂S₂, or N₄ chelators (see also, U.S. Pat. No. 4,647,447, U.S. Pat. No. 4,957,939, U.S. Pat. No. 4,963,344, U.S. Pat. No. 5,367,080, U.S. Pat. No. 5,364,613, U.S. Pat. No. 5,021,556, U.S. Pat. No. 5,075,099, U.S. Pat. No. 5,886,142, the disclosures of which are incorporated by reference herein in their entirety), and other chelators known in the art including, but not limited to, HYNIC, DTPA, EDTA, DOTA, TETA, and bisamino bisthiol (BAT) chelators (see also U.S. Pat. No. 5,720,934). For example, macrocyclic chelators, and in particular N₄ chelators are described in U.S. Pat. Nos. 4,885,363; 5,846,519; 5,474,756; 6,143,274; 6,093,382; 5,608,110; 5,665,329; 5,656,254; and 5,688,487, the disclosures of which are incorporated by reference herein in their entirety. Certain N₃S chelators are described in PCT/CA94/00395, PCT/CA94/00479, PCT/CA95/00249 and in U.S. Pat. Nos. 5,662,885; 5,976,495; and 5,780,006, the disclosures of which are incorporated by reference herein in their entirety. The chelator may also include derivatives of the chelating ligand mercapto-acetyl-glycyl-glycyl-glycine (MAG3), which contains an N₃S, and N₂S₂ systems such as MAMA (monoamidemonoaminedithiols), DADS (N₂S diaminedithiols), CODADS and the like. These ligand systems and a variety of others are described in Liu and Edwards, Chem Rev. 1999, 99, 2235-2268; Caravan et al., Chem. Rev. 1999, 99, 2293-2352; and references therein, the disclosures of which are incorporated by reference herein in their entirety.

The metal chelator may also include complexes known as boronic acid adducts of technetium and rhenium dioximes, such as those described in U.S. Pat. Nos. 5,183,653; 5,387,409; and 5,118,797, the disclosures of which are incorporated by reference herein, in their entirety.

Examples of preferred chelators include, but are not limited to, derivatives of diethylenetriamine pentaacetic acid (DTPA), 1,4,7,10-tetraazacyclotetradecane- 1,4,7,10-tetraacetic acid (DOTA), 1-substituted 1,4,7,-tricarboxymethyl 1,4,7,10 tetraazacyclododecane triacetic acid (DO3A), derivatives of the 1-1-(1-carboxy-3-(p-nitrophenyl)propyl-1,4,7,10 tetraazacyclododecane triacetate (PA-DOTA) and MeO-DOTA, ethylenediaminetetraacetic acid (EDTA), and 1,4,8,11-tetraazacyclotetradecane-1,4,8,11-tetraacetic acid (TETA), derivatives of 3,3,9,9-Tetramethyl-4,8-diazaundecane-2,10-dione dioxime (PnAO); and derivatives of 3,3,9,9-Tetramethyl-5-oxa-4,8-diazaundecane-2,10-dione dioxime (oxa PnAO). Additional chelating ligands are ethylenebis-(2-hydroxy-phenylglycine) (EHPG), and derivatives thereof, including 5-Cl-EHPG, 5-Br-EHPG, 5-Me-EHPG, 5-t-Bu-EHPG, and 5-sec-Bu-EHPG; benzodiethylenetriamine pentaacetic acid (benzo-DTPA) and derivatives thereof, including dibenzo-DTPA, phenyl-DTPA, diphenyl-DTPA, benzyl-DTPA, and dibenzyl-DTPA; bis-2 (hydroxybenzyl)-ethylene-diaminediacetic acid (HBED) and derivatives thereof; the class of macrocyclic compounds which contain at least 3 carbon atoms, more preferably at least 6, and at least two heteroatoms (O and/or N), which macrocyclic compounds can consist of one ring, or two or three rings joined together at the hetero ring elements, e.g., benzo-DOTA, dibenzo-DOTA, and benzo-NOTA, where NOTA is 1,4,7-triazacyclononane N,N′,N″-triacetic acid, benzo-TETA, benzo-DOTMA, where DOTMA is 1,4,7,10-tetraazacyclotetradecane-1,4,7,10-tetra(methyl tetraacetic acid), and benzo-TETMA, where TETMA is 1,4,8,11- tetraazacyclotetradecane-1,4,8,1 1-(methyl tetraacetic acid); derivatives of 1,3-propylenediaminetetraacetic acid (PDTA) and triethylenetetraaminehexaacetic acid (TTHA); derivatives of 1,5,10-N,N′,N″-tris(2,3-dihydroxybenzoyl)-tricatecholate (LICAM) and 1,3,5-N,N′,N″-tris(2,3-dihydroxybenzoyl) aminomethylbenzene (MECAM). Examples of representative chelators and chelating groups contemplated by the present invention are described in WO 98/18496, WO 86/06605, WO 91/03200, WO 95/28179, WO 96/23526, WO 97/36619, PCT/US98/01473, PCT/US98/20182, and U.S. Pat. No. 4,899,755, U.S. Pat. No. 5,474,756, U.S. Pat. No. 5,846,519 and U.S. Pat. No. 6,143,274, each of which is hereby incorporated by reference in its entirety.

Particularly preferred metal chelators include those of Formula 1, 2 and 3a and 3b (for ¹¹¹In, ⁹⁰Y, and radioactive lanthanides, such as, for example ¹⁷⁷Lu, ¹⁵³Sm, and ¹⁶⁶Ho) and those of Formula 4, 5 and 6 (for radioactive ^{99m}Tc, ¹⁸⁶Re, and ¹⁸⁸Re) set forth below. These and other metal chelating groups are described in U.S. Pat. Nos. 6,093,382 and 5,608,110, which are incorporated by reference herein in their entirety. Additionally, the chelating group of Formula 3 is described in, for example, U.S. Pat. No. 6,143,274; the chelating group of Formula 5 is described in, for example, U.S. Pat. Nos. 5,627,286 and 6,093,382, and the chelating group of Formula 6 is described in, for example, U.S. Pat. Nos. 5,662,885; 5,780,006; and 5,976,495, all of which are incorporated by reference. Specific metal chelators of Formula 6 include N,N-dimethylGly-Ser-Cys; N,N-dimethylGly-Thr-Cys; N,N-diethylGly-Ser-Cys; N,N-dibenzylGly-Ser-Cys- and other variations thereof. Spacers which do not actually complex with the metal radionuclide such as an extra single amino acid Gly, may be attached to these metal chelators (e.g., N,N-dimethylGly-Ser-Cys-Gly; N,N-dimethylGly-Thr-Cys-Gly; N,N-diethylGly-Ser-Cys-Gly; N,N-dibenzylGly-Ser-Cys-Gly). Other useful metal chelators such as all of those disclosed in U.S. Pat. No. 6,334,996, are also incorporated by reference (e.g., Dimethylgly-L-t-Butylgly-L-Cys-Gly; Dimethylgly-D-t-Butylgly-L-Cys-Gly; Dimethylgly-L-t-Butylgly-L-Cys, etc.).

Furthermore, sulfur protecting groups such as Acm (acetamidomethyl), trityl or other known alkyl, aryl, acyl, alkanoyl, aryloyl, mercaptoacyl and organothiol groups may be attached to the cysteine amino acid of these metal chelators.

In particular, useful metal chelators include:

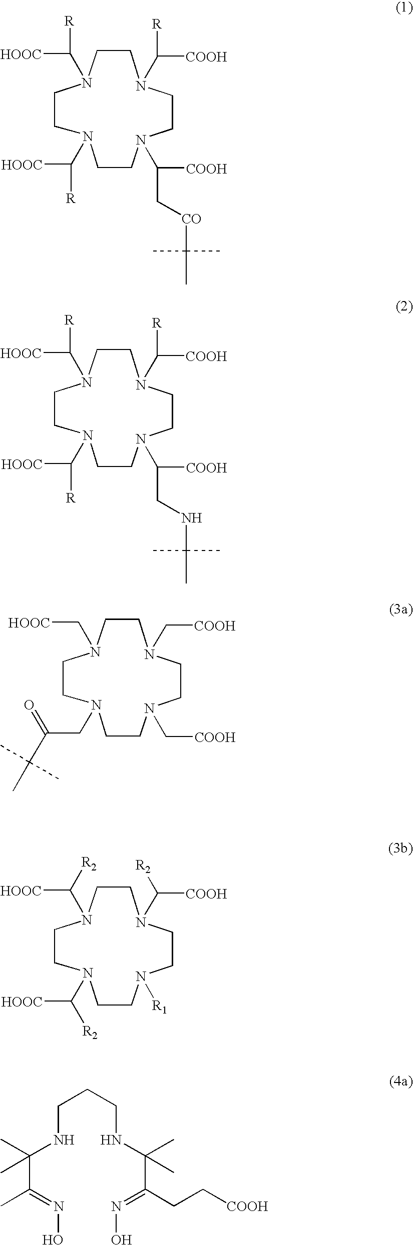

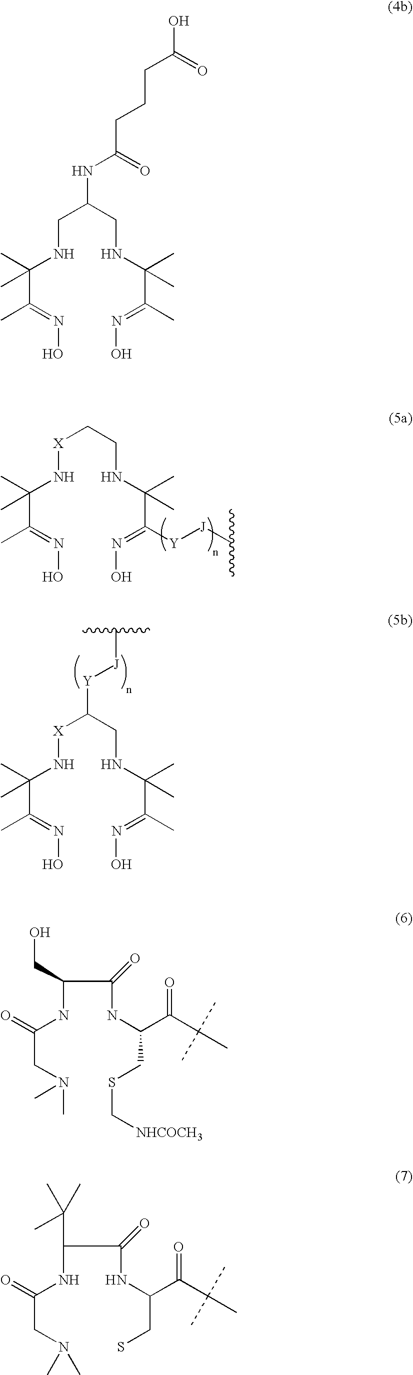

In the above Formulas 1 and 2, R is hydrogen or alkyl, preferably methyl. In Formula 3b, R₁ and R₂ are as defined in U.S. Pat. No. 6,143,274, incorporated by reference herein its entirety. In the above Formula 5, X is either CH₂ or O, Y is either C₁-C₁₀ branched or unbranched alkyl; Y is aryl, aryloxy, arylamino, arylaminoacyl; Y is arylalkyl—where the alkyl group or groups attached to the aryl group are C₁-C₁₀ branched or unbranched alkyl groups, C₁-C₁₀ branched or unbranched hydroxy or polyhydroxyalkyl groups or polyalkoxyalkyl or polyhydroxy-polyalkoxyalkyl groups, J is C(═O)—, OC(═O)—, SO₂—, NC(═O)—, NC(═S)—, N(Y), NC(═NCH₃)—, NC(═NH)—, N═N—, homopolyamides or heteropolyamines derived from synthetic or naturally occurring amino acids; all where n is 1-100. J may also be absent. Other variants of these structures are described, for example, in U.S. Pat. No. 6,093,382. In Formula 6, the group S—NHCOCH₃ may be replaced with SH or S-Z wherein Z is any of the known sulfur protecting groups such as those described above. Formula 7 illustrates one embodiment of t-butyl compounds useful as a metal chelator. The disclosures of each of the foregoing patents, applications and references are incorporated by reference herein, in their entirety.

In a preferred embodiment, the metal chelator includes cyclic or acyclic polyaminocarboxylic acids such as DOTA (1,4,7,10-tetraazacyclododecane -1,4,7,10-tetraacetic acid), DTPA (diethylenetriaminepentaacetic acid), DTPA-bismethylamide, DTPA-bismorpholineamide, DO3A N-[[4,7,10-Tris(carboxymethyl)-1,4,7,10-tetraazacyclododec-1-yl]acetyl], HP-DO3A, DO3A-monoamide and derivatives thereof.

These chelating ligands encapsulate the radiometal by binding to it via multiple nitrogen and oxygen atoms, thus preventing the release of free (unbound) radiometal into the body. This is important, as in- vivo dissociation of 3+radiometals from their chelate can result in uptake of the radiometal in the liver, bone and spleen [Brechbiel M W, Gansow O A, “Backbone-substituted DTPA ligands for ⁹⁰Y radioimmunotherapy”, Bioconj. Chem. 1991; 2:187-194; Li, W P, Ma D S, Higginbotham C, Hoffman T, Ketring A R, Cutler C S, Jurisson, S S, “Development of an in vitro model for assessing the in vivo stability of lanthanide chelates.” Nucl. Med. Biol. 2001; 28(2): 145-154; Kasokat T, Urich K. Arzneim.-Forsch, “Quantification of dechelation of gadopentetate dimeglumine in rats.” 1992; 42(6): 869-76]. Unless one is specifically targeting these organs, such non-specific uptake is highly undesirable, as it leads to non-specific irradiation of non-target tissues, which can lead to such problems as hematopoietic suppression due to irradiation of bone marrow.

### 2. Radioisotopes

Preferred radionuclides for scintigraphy or radiotherapy include ^{99m}Tc, ⁶⁷Ga, ⁶⁸Ga, ⁴⁷Sc, ⁵Cr, ¹⁶⁷Tm, ¹⁴¹Ce, ¹¹¹In, ¹²³I, ¹²⁵I, ¹³¹I, ¹⁸F, ¹⁸F, ¹¹C, ¹⁵N, ¹⁶⁸Yb, ¹⁷⁵Yb, ¹⁴⁰La, ⁹⁰Y, ⁸⁸Y′, ⁸⁶Y′ ¹⁵³Sm, ¹⁶⁶Ho, ¹⁶⁵Dy, ¹⁶⁶Dy, ⁶²Cu, ⁶⁴Cu, ⁶⁷Cu, ⁹⁷Ru, ¹⁰³Ru, ¹⁸⁶Re, ¹⁸⁸Re, ²⁰³_l Pb, ²¹¹Bi, ²¹²Bi, ²¹³Bi, ²¹⁴Bi, ²²⁵Ac, ²¹¹At, ¹⁰⁵Rh, ¹⁰⁹Pd, ^{117m}Sn, ¹⁴⁹Pm ¹⁶¹Tb, ¹⁷⁷Lu, ¹⁹⁸Au, ¹⁹⁹Au, and oxides or nitrides thereof. The choice of isotope will be determined based on the desired therapeutic or diagnostic application. For example, for diagnostic purposes (e.g., to diagnose and monitor therapeutic progress in primary tumors and metastases), the preferred radionuclides include ⁶⁴Cu, ⁶⁷Ga, ⁶⁸Ga, ^{99m}Tc, and ¹¹¹In, with ^{99m}Tc and ¹¹¹In being especially preferred. For therapeutic purposes (e.g., to provide radiotherapy for primary tumors and metastasis related to cancers of the prostate, breast, lung, etc.), the preferred radionuclides include ⁶⁴Cu, ⁹⁰Y, ¹⁰⁵Rh, ¹¹¹In, ^{117m}Sn, ¹⁴⁹Pm, ¹⁵³Sm, ¹⁶¹Tb, ¹⁶⁶Dy, ¹⁶⁶Ho, ¹⁷⁵Yb, ¹⁷⁷Lu, ^{186/188}Re, and ¹⁹⁹Au, with ¹⁷⁷Lu and ⁹⁰Y being particularly preferred. ^{99m}Tc is particularly useful and is a preferred diagnostic radionuclide because of its low cost, availability, imaging properties, and high specific activity. The nuclear and radioactive properties of ^{99m}Tc make this isotope an ideal scintigraphic imaging agent. This isotope has a single photon energy of 140 keV and a radioactive half-life of about 6 hours, and is readily available from a ⁹⁹Mo-^{99m}Tc generator. ¹¹¹In is also particularly preferred diagnostic isotope, as this +3 metal ion has very similar chemistry to that of the radiotherapeutic +3 lanthanides, thus allowing the preparation of a diagnostic/therapeutic ¹¹¹In/¹⁷⁷Lu pair. Peptides labeled with ¹⁷⁷Lu, ⁹⁰Y or other therapeutic radionuclides can be used to provide radiotherapy for primary tumors and metastasis related to cancers of the prostate, breast, lung, etc., and ¹¹¹In analogs can be used to detect the presence of such tumors. The selection of a proper nuclide for use in a particular radiotherapeutic application depends on many factors, including:

a. Physical half-life—This should be long enough to allow synthesis and purification of the radiotherapeutic construct from radiometal and conjugate, and delivery of said construct to the site of injection, without significant radioactive decay prior to injection. Preferably, the radionuclide should have a physical half-life between about 0.5 and 8 days.

b. Energy of the emission(s) from the radionuclide—Radionuclides that are particle emitters (such as alpha emitters and beta emitters) are particularly useful, as they emit highly energetic particles that deposit their energy over short distances, thereby producing highly localized damage. Beta emitting radionuclides are particularly preferred, as the energy from beta particle emissions from these isotopes is deposited within 5 to about 150 cell diameters. Radiotherapeutic agents prepared from these nuclides are capable of killing diseased cells that are relatively close to their site of localization, but cannot travel long distances to damage adjacent normal tissue such as bone marrow.

c. Specific activity (i.e. radioactivity per mass of the radionuclide)—Radionuclides that have high specific activity (e.g. generator produced 90-Y, 111-In, 177-Lu) are particularly preferred. The specific activity of a radionuclide is determined by its method of production, the particular target that is used to produce it, and the properties of the isotope in question.

3. Linking Groups

The terms “linker,” and “linking group” are used synonymously herein to refer to any chemical group that serves to couple the targeting molecule to the metal chelator while not adversely affecting either the targeting function of the targeting molecule or the metal complexing function of the metal chelator. Linking groups may optionally be present in the stabilized radiopharmaceutical formulations of the invention.

Suitable linking groups include peptides (i.e., amino acids linked together) alone, a non-peptide group (e.g., hydrocarbon chain) or a combination of an amino acid sequence and a non-peptide spacer.

In one embodiment, the linking group includes L-glutamine and a hydrocarbon chain, or a combination thereof.

In another embodiment, the linking group includes a pure peptide linking group consisting of a series of amino acids (e.g., diglycine, triglycine, gly-gly-glu, gly-ser-gly, etc.), in which the total number of atoms between the N-terminal residue of the targeting molecule and the metal chelator in the polymeric chain is ≦12 atoms.

In yet a further embodiment, the linking group includes a hydrocarbon chain [i.e., R₁—(CH₂)ₙ—R₂] wherein n is 0-10, preferably n=3 to 9, R₁ is a group (e.g., H₂N—, HS—, —COOH) that can be used as a site for covalently linking the ligand backbone or the preformed metal chelator or metal complexing backbone; and R₂ is a group that is used for covalent coupling to the targeting molecule (e.g., to the N-terminal NH₂-group of a targeting peptide (e.g., R₂ is an activated COOH group)). Several chemical methods for conjugating ligands (i.e., chelators) or preferred metal chelates to biomolecules have been well described in the literature [Wilbur, 1992; Parker, 1990; Hermanson, 1996; Frizberg et al., 1995]. One or more of these methods could be used to link either the uncomplexed ligand (chelator) or the radiometal chelate to the linker or to link the linker to the targeting molecule. These methods include the formation of acid anhydrides, aldehydes, arylisothiocyanates, activated esters, or N-hydroxysuccinimides [Wilbur, 1992; Parker, 1990; Hermanson, 1996; Frizberg et al., 1995].

3A. Linking Groups Containing At Least One Non-Alpha Amino Acid

In a preferred embodiment of the invention, the linking group is of the formula N—O—P and contains at least one non-alpha amino acid. Thus, in this embodiment of the linker N—O—P,
N is 0 (where 0 means it is absent), an alpha or non-alpha amino acid or other linking group;
O is an alpha or non-alpha amino acid; and
P is 0, an alpha or non-alpha amino acid or other linking group,
wherein at least one of N, O or P is a non-alpha amino acid.

Thus, in one example, N=Gly, O=a non-alpha amino acid, and P=0.

Alpha amino acids are well known in the art, and include naturally occurring and synthetic amino acids. Non-alpha amino acids also include those which are naturally occurring or synthetic. Preferred non-alpha amino acids include:
8-amino-3,6-dioxaoctanoic acid;
N-4-aminoethyl-N-1-acetic acid; and
polyethylene glycol derivatives having the formula NH₂—(CH₂CH₂O)n—CH₂CO₂H or NH₂—(CH₂CH₂O)n—CH₂CH₂CO₂H where n=2 to 100.

3B. Linking Groups Containing At Least One Substituted Bile Acid

In another embodiment of the present invention, the linker is of the formula N—O—P and contains at least one substituted bile acid. Thus, in this embodiment of the linker N—O—P,
N is 0 (where 0 means it is absent), an alpha amino acid, a substituted bile acid or other linking group;
O is an alpha amino acid or a substituted bile acid; and
P is 0, an alpha amino acid, a substituted bile acid or other linking group,
wherein at least one of N, O or P is a substituted acid.

Bile acids are found in bile (a secretion of the liver) and are steroids having a hydroxyl group and a five carbon atom side chain terminating in a carboxyl group. In substituted bile acids, at least one atom such as a hydrogen atom of the bile acid is substituted with another atom, molecule or chemical group. For example, substituted bile acids include those having a 3-amino, 24-carboxyl function optionally substituted at positions 7 and 12 with hydrogen, hydroxyl or keto functionality.

Other useful substituted bile acids in the present invention include substituted cholic acids and derivatives thereof. Specific substituted cholic acid derivatives include:
(3β,5β)-3-aminocholan-24-oic acid;
(3β,5β,12α)-3-amino-12-hydroxycholan-24-oic acid;
(3β,5β,7α,12α)-3-amino-7,12-dihydroxycholan-24-oic acid;
Lys-(3,6,9)-trioxaundecane-1,11-dicarbonyl-3,7-dideoxy-3-aminocholic acid);
(3β,5β,7α)-3-amino-7-hydroxy-12-oxocholan-24-oic acid; and
(3β,5β,7α)-3-amino-7-hydroxycholan-24-oic acid.

3C. Linkers Containing at Least One Non-Alpha Amino Acid With a Cyclic Group

In yet another embodiment of the present invention, the linker N—O—P contains at least one non-alpha amino acid with a cyclic group. Thus, in this embodiment of the linker N—O—P,
N is 0 (where 0 means it is absent), an alpha amino acid, a non-alpha amino acid with a cyclic group or other linking group;
O is an alpha amino acid or a non-alpha amino acid with a cyclic group; and
P is 0, an alpha amino acid, a non-alpha amino acid with a cyclic group, or other linking group,
wherein at least one of N, O or P is a non-alpha amino acid with a cyclic group.

Non-alpha amino acids with a cyclic group include substituted phenyl, biphenyl, cyclohexyl or other amine and carboxyl containing cyclic aliphatic or heterocyclic moieties. Examples of such include:
4-aminobenzoic acid
4-aminomethyl benzoic acid
trans-4-aminomethylcyclohexane carboxylic acid
4-(2-aminoethoxy)benzoic acid
isonipecotic acid
2-aminomethylbenzoic acid
4-amino-3-nitrobenzoic acid
4-(3-carboxymethyl-2-keto-1-benzimidazolyl-piperidine
6-(piperazin-1-yl)-4-(3H)-quinazolinone-3-acetic acid
(2S,5S)-5-amino-1,2,4,5,6,7-hexahydro-5-amino-1,2,4,5,6,7-hexahydro azepino[3,2,1-hi]indole-4-one-2-carboxylic acid
(4S,7R)-4-amino-6-aza-5-oxo-9-thiabicyclo[4.3.0]nonane-7-carboxylic acid
3-carboxymethyl-1-phenyl-1,3,8-triazaspiro[4.5]decan-4-one
N1-piperazineacetic acid
N-4-aminoethyl-N-1-piperazineacetic acid
(3S)-3-amino-1-carboxymethylcaprolactam
(2S,6S,9)-6-amino-2-carboxymethyl-3,8-diazabicyclo-[4,3,0]-nonane-1,4-dione

4. Targeting Molecule

Any molecule that specifically binds to or reactively associates or complexes with a receptor or other receptive moiety associated with a given target cell population may be used as a targeting molecule in radiopharmaceutical formulations of the invention. This cell reactive molecule, to which the metal chelator is linked optionally via a linking group, may be any molecule that binds to, complexes with or reacts with the cell population sought to be bound or localized to. The cell reactive molecule acts to deliver the radiopharmaceutical to the particular target cell population with which the molecule reacts. The targeting molecule may be non-peptidic such as, for example, steroids, carbohydrates, or small non-peptidic molecules. The targeting molecule may also be an antibody, such as, for example, a monoclonal or polyclonal antibody, a fragment thereof, or a protein, including, for example, derivatives of Annexin, anti-CEA, Tositumomab, HUA33, Epratuzumab, cG250, human serum albumin, Ibritumomab Tiuxetan and the like. Preferably the targeting molecule is a peptide, peptide mimetic or peptoid. Most preferably the targeting molecule is a peptide (a “targeting peptide”).

In preferred embodiments, the targeting molecule used in a radiopharmaceutical formulation of the invention is a biologically active peptide.

In a more preferred embodiment, the targeting molecule is a peptide that binds to a receptor or enzyme of interest. For example, the targeting molecule may be a peptide hormone such as, for example, luteinizing hormone releasing hormone (LHRH) such as that described in the literature (e.g., Radiometal-Binding Analogues of Luteinizing Hormone Releasing Hormone PCT/US96/08695; PCT/US97/12084 (WO 98/02192)); insulin; oxytocin; somatostatin; Neuro kinin-1 (NK-1); Vasoactive Intestinal Peptide (VIP) including both linear and cyclic versions as delineated in the literature, [e.g., Comparison of Cyclic and Linear Analogs of Vasoactive Intestinal Peptide. D. R. Bolin, J. M. Cottrell, R. Garippa, N. Rinaldi, R. Senda, B. Simkio, M. O'Donnell. Peptides: Chemistry, Structure and Biology Pravin T. P. Kaumaya, and Roberts S. Hodges (Eds). Mayflower Scientific LTD., 1996, pgs 174-175]; gastrin releasing peptide (GRP); bombesin and other known hormone peptides, as well as analogues and derivatives thereof.

Other useful targeting molecules include analogues of somatostatin which, for example, are Lanreotide (Nal-Cys-Thr-DTrp-Lys-Val-Cys-Thr-NH₂), Octreotide (Nal-Cys-Thr-DTrp-Lys-Val-Cys-Thr-ol), and Maltose-(Phe-Cys-Thr-DTrp-Lys-Val-Cys-Thr-ol). These analogues are described in the literature [e.g., Potent Somatostatin Analogs Containing N-terminal Modifications, S. H. Kim, J. Z. Dong, T. D. Gordon, H. L. Kimball, S. C. Moreau, J.-P. Moreau, B. A. Morgan, W. A. Murphy and J. E. Taylor; Peptides: Chemistry, Structure and Biology Pravin T. P. Kaumaya, and Roberts S. Hodges (Eds)., Mayflower Scientific LTD., 1996, pgs 241-243.]

Still other useful targeting molecules include Substance P agonists [e.g., G. Bitan, G. Byk, Y. Mahriki, M. Hanani, D. Halle, Z. Selinger, C. Gilon, Peptides: Chemistry, Structure and Biology, Pravin T. P. Kaumaya, and Roberts S. Hodges (Eds), Mayflower Scientific LTD., 1996, pgs 697-698; G Protein Antagonists A novel hydrophobic peptide competes with receptor for G protein binding, Hidehito Mukai, Eisuke Munekata, Tsutomu Higashijima, J. Biol. Chem. 1992, 267, 16237-16243]; NPY(Y1) [e.g., Novel Analogues of Neuropeptide Y with a Preference for the Y1 -receptor, Richard M. Soll, Michaela, C. Dinger, Ingrid Lundell, Dan Larhammer, Annette G. Beck-Sickinger, Eur. J. Biochem. 2001, 268, 2828-2837; ^{99m}Tc-Labeled Neuropeptide Y Analogues as Potential Tumor Imaging Agents, Michael Langer, Roberto La Bella, Elisa Garcia-Garayoa, Annette G. Beck-Sickinger, Bioconjugate Chem. 2001, 12, 1028-1034; Novel Peptide Conjugates for Tumor-Specific Chemotherapy, Michael Langer, Felix Kratz, Barbara Rothen-Rutishauser, Heidi Wnderli-Allenspach, Annette G. Beck-Sickinger, J. Med. Chem. 2001, 44, 1341-1348]; oxytocin; endothelin A and endothelin B; bradykinin; Epidermal Growth Factor (EGF); Interleukin-1 [Anti-IL-1 Activity of Peptide Fragments of IL-1 Family Proteins, I. Z. Siemion, A. Kluczyk, Zbigtniew Wieczorek, Peptides 1998, 19, 373-382]; and cholecystokinin (CCK-B) [Cholecystokinin Receptor Imaging Using an Octapeptide DTPA-CCK Analogue in Patients with Medullary Thryroid Carcinoma, Eur. J. Nucl Med. 200, 27, 1312-1317]. Other useful as targeting molecules include: transferrin, platelet-derived growth factor, tumor growth factors (“TGF”), such as TGF-a and TGF-β, vaccinia growth factor (“VGF”), insulin-like growth factors I and II, urotensin II peptides and analogs, depreotide, vapreotide, insulinlike growth factor (IGF), peptides targeting receptors which are upregulated in angiogenesis such as VEGF receptors (e.g., KDR, NP-1, etc.), RGD-containing peptides, melanocyte-stimulating hormone (MSH) peptide, neurotensin, calcitonin, peptides from complementarity determining regions of an antitumor antibody, glutathione, YIGSR (leukocyte-avid peptides, e.g., P483H, which contains the heparin-binding region of platelet factor-4 (PF-4) and a lysine-rich sequence), atrial natriuretic peptide (ANP), β-amyloid peptides, delta-opioid antagonists (such as ITIPP(psi)), annexin-V, IL-1/IL-1ra, IL-2, IL-6, IL-8, leukotriene B4 (LTB4), chemotactic peptides (such as N-formyl-methionyl-leucyl-phenylalanine-lysine (fMLFK)), GP IIb/IIIa receptor antagonists (such as DMP444), epidermal growth factor, human neutrophil elastase inhibitor (EPI-HNE-2, HNE2, and HNE4), plasmin inhibitor, antimicrobial peptides, apticide (P280), P274, thrombospondin receptor (including analogs such as TP-1300), bitistatin, pituitary adenyl cyclase type I receptor (PAC1), and analogues and derivatives of these.

A general review of targeting molecules, can be found, for example, in the following: The Role of Peptides and Their Receptors as Tumor Markers, Jean-Claude Reubi, Gastrointestinal Hormones in Medicine, pg. 899-939; Peptide Radiopharmaceuticals in Nuclear Medicine, D. Blok, R. I. J. Feitsma, P. Vermeij, E. J. K. Pauwels, Eur. J. Nucl Med. 1999, 26, 1511-1519; and Radiolabeled Peptides and Other Ligands for Receptors Overexpressed in Tumor Cells for Imaging Neoplasms, John G. McAfee, Ronald D. Neumann, Nuclear Medicine and Biology, 1996, 23, 673-676 (somatostatin, VIP, CCK, GRP, Substance P, Galanin, MSH, LHRH, Arginine-vasopressin, endothelin). All of the aforementioned literature in the preceding paragraphs are herein incorporated by reference in their entirety.

Other targeting molecule references, include the following: Co-expressed peptide receptors in breast cancer as a molecular basis of in vivo multireceptor tumour targeting. Jean Claude Reubi, Mathias Gugger, Beatrice Waser. Eur. J. Nucl Med. 2002, 29, 855-862, (includes NPY, GRP); Radiometal-Binding Analogues of Leutenizing Hormone Releasing Hormone PCT/US96/08695 (LHRH); PCT/US97/12084 (WO 98/02192) (LHRH); PCT/EP90/01169 (radiotherapy of peptides); WO 91/01144 (radiotherapy of peptides); and PCT/EP00/01553 (molecules for the treatment and diagnosis of tumours), all of which are herein incorporated by reference in their entirety.

Additionally, analogues of a targeting molecule can be used. These analogues include molecules that target a desired site receptor with avidity that is greater than or equal to the targeting molecule itself. For targeting peptides analogues include muteins, retropeptides and retro-inverso-peptides of the targeting peptide. One of ordinary skill will appreciate that these analogues may also contain modifications which include substitutions, and/or deletions and/or additions of one or several amino acids, insofar that these modifications do not negatively alter the biological activity of the targeting molecules. Substitutions in targeting peptides may be carried out by replacing one or more amino acids by their synonymous amino acids. Synonymous amino acids within a group are defined as amino acids that have sufficient physicochemical properties to allow substitution between members of a group in order to preserve the biological function of the molecule. Synonymous amino acids as used herein include synthetic derivatives of these amino acids (such as for example the D-forms of amino acids and other synthetic derivatives), and, the D-forms of amino acids and other synthetic derivatives). Throughout this application amino acids are abbreviated interchangeably either by their three letter or single letter abbreviations, which are well known to the skilled artisan. Thus, for example, T or Thr stands for threonine, K or Lys stands for lysine, P or Pro stands for proline and R or Arg stands for arginine.

Deletions or insertions of amino acids may also be introduced into the defined sequences of targeting peptides provided they do not alter the biological functions of said sequences. Preferentially such insertions or deletions should be limited to 1, 2, 3, 4 or 5 amino acids and should not remove or physically disturb or displace amino acids which are critical to the functional conformation. Muteins of targeting peptides or polypeptides may have a sequence homologous to the original targeting peptide sequence in which amino acid substitutions, deletions, or insertions are present at one or more amino acid positions. Muteins may have a biological activity that is at least 40%, preferably at least 50%, more preferably 60-70%, most preferably 80-90% of the original targeting peptide. However, they may also have a biological activity greater than the original targeting peptide, and thus do not necessarily have to be identical to the biological function of the original targeting peptides. Analogues of targeting peptides also include peptidomimetics or pseudopeptides incorporating changes to the amide bonds of the peptide backbone, including thioamides, methylene amines, and E-olefins. Also molecules based on the structure of a targeting peptide or its analogues with amino acids replaced by N-substituted hydrazine carbonyl compounds (also known as aza amino acids) are included in the term analogues as used herein.

Where a targeting peptide is used, it may be attached to a linker via the N or C terminus or via attachment to the epsilon nitrogen of lysine, the gamma nitrogen or ornithine or the second carboxyl group of aspartic or glutamic acid.

In a preferred embodiment, the targeting molecule is a gastrin releasing peptide (GRP) receptor targeting molecule. A GRP receptor-targeting molecule is a molecule that specifically binds to or reactively associates or complexes with one or more members of the GRP receptor family. In other words, it is a molecule which has a binding affinity for the GRP receptor family. In an especially preferred embodiment, the targeting molecule is a GRP receptor targeting peptide (e.g., a peptide, equivalent, analogue or derivative thereof with a binding affinity for one or more members of the GRP receptor family).

The GRP receptor targeting molecule may take the form of an agonist or an antagonist. A GRP receptor targeting molecule agonist is known to “activate” the cell following binding with high affinity and may be internalized by the cell. Conversely, GRP receptor targeting molecule antagonists are known to bind only to the GRP receptor on the cell without stimulating internalization by the cell and without “activating” the cell. In a preferred embodiment, the GRP receptor targeting molecule is an agonist and more preferably it is a peptide agonist.

In a more preferred embodiment of the present invention, the GRP agonist is a bombesin (BBN) analogue and/or a derivative thereof. The BBN derivative or analog thereof preferably contains either the same primary structure of the BBN binding region (i.e., BBN(7-14) [SEQ ID NO:1]) or similar primary structures, with specific amino acid substitutions that will specifically bind to GRP receptors with better or similar binding affinities as BBN alone (i.e., Kd<25 nM). Suitable compounds include peptides, peptidomimetics and analogues and derivatives thereof. The presence of L-methionine (Met) at position BBN-14 will generally confer agonistic properties while the absence of this residue at BBN-14 generally confers antagonistic properties [Hofflen, 1994].

It is well documented in the art that there are a few and selective number of specific amino acid substitutions in the BBN (8-14) binding region (e.g., D-Ala¹¹ for L-Gly¹¹ or D-Trp⁸ for L-Trp⁸), which can be made without decreasing binding affinity [Leban et al., 1994; Qin et al., 1994; Jensen et al., 1993]. In addition, attachment of some amino acid chains or other groups to the N-terminal amine group at position BBN-8 (i.e., the Trp⁸ residue) can dramatically decrease the binding affinity of BBN analogues to GRP receptors [Davis et al., 1992; Hoffken, 1994; Moody et al., 1996; Coy, et al., 1988; Cai et al., 1994]. In a few cases, it is possible to append additional amino acids or chemical moieties without decreasing binding affinity.

Analogues of BBN receptor targeting molecules include molecules that target the GRP receptors with avidity that is greater than or equal to BBN, as well as muteins, retropeptides and retro-inverso-peptides of GRP or BBN. One of ordinary skill will appreciate that these analogues may also contain modifications which include substitutions, and/or deletions and/or additions of one or several amino acids, insofar that these modifications do not negatively alter the biological activity of the peptides described therein. These substitutions may be carried out by replacing one or more amino acids by their synonymous amino acids.

The stabilizers of the present invention may also be used for compounds that do not have a distinct targeting or linking group, and wherein the metal/chelator combination alone provides targeting to the desired organ or organ system. For example, the stabilizers described here have potential utility in the stabilization of compounds such as ¹⁶⁶Ho-DOTMP, ¹⁸⁸Re-HEDTMP, ¹⁵³Sm-EDTMP, ^{99m}Tc-MDP and the like, all of which target bone.

5. Labeling and Administration of Compounds

Incorporation of the radioisotope within the stabilized conjugates of this invention can be achieved by various methods commonly known in the art of coordination chemistry. Where incorporation of, for example, ¹¹¹In or ¹⁷⁷Lu is desired, the methods set forth in the Examples may be used. When the metal is ^{99m}Tc, a preferred radionuclide for diagnostic imaging, the following general procedure can be used to form a technetium complex. A peptide-chelator conjugate solution is formed by initially dissolving the conjugate in an aqueous solution of dilute acid, base, salt or buffer, or in an aqueous solution of an alcohol such as ethanol. The solution is then optionally degassed to remove dissolved oxygen. When an —SH group is present in the peptide, a thiol protecting group such as Acm (acetamidomethyl), trityl or other thiol protecting group may optionally be used to protect the thiol from oxidation. The thiol protecting group(s) are removed with a suitable reagent, for example with sodium hydroxide, and are then neutralized with an organic acid such as acetic acid. Alternatively, the thiol protecting group can be removed in situ during technetium chelation. In the labeling step, sodium pertechnetate obtained from a molybdenum generator is added to a solution of the conjugate with a sufficient amount of a reducing agent, such as stannous chloride, to reduce technetium and is either allowed to stand at room temperature or is heated. The labeled conjugate can be separated from the contaminants ^{99m}TcO₄ and colloidal ^{99m}TcO₂ chromatographically, for example with a C-18 Sep Pak cartridge [Millipore Corporation] or by HPLC using methods known to those skilled in the art.

In an alternative method, the labeling can be accomplished by a transchelation reaction. In this method, the technetium source is a solution of technetium that is reduced and complexed with labile ligands prior to reaction with the selected chelator, thus facilitating ligand exchange with the selected chelator. Examples of suitable ligands for transchelation includes tartrate, citrate, gluconate, and heptagluconate. It will be appreciated that the conjugate can be labeled using the techniques described above, or alternatively, the chelator itself may be labeled and subsequently coupled to the peptide to form the conjugate; a process referred to as the “prelabeled chelate” method. Re and Tc are both in row VIIB of the Periodic Table and they are chemical congeners. Thus, for the most part, the complexation chemistry of these two metals with ligand frameworks that exhibit high in vitro and in vivo stabilities are the same [Eckelman, 1995] and similar chelators and procedures can be used to label with Re. Many ^{99m}Tc or ^{186/188}Re complexes, which are employed to form stable radiometal complexes with peptides and proteins, chelate these metals in their +5 oxidation state [Lister-James et al., 1997]. This oxidation state makes it possible to selectively place ^{99m}Tc- or ^{186/188}Re into ligand frameworks already conjugated to the biomolecule, constructed from a variety of ^{99m}Tc(V) and/or ^{186/188}Re(V) weak chelates (e.g., ^{99m}Tc- glucoheptonate, citrate, gluconate, etc.) [Eckelman, 1995; Lister-James et al., 1997; Pollak et al., 1996].

6. Diagnostic and Therapeutic Uses

The stabilized radiopharmaceuticals and radiopharmaceutical formulations of the present invention can be used to image or deliver radiotherapy to selected tissues. In a preferred embodiment, they may be used to treat and/or detect cancers, including tumors, by procedures established in the art of radiodiagnostics and radiotherapeutics. [Bushbaum, 1995; Fischman et al., 1993; Schubiger et al., 1996; Lowbertz et al., 1994; Krenning et al., 1994].

Indeed the stabilized radiopharmaceutical formulations of the examples are able to target GRP receptor expressing tissues, including tumors and thus to image or deliver radiotherapy to these tissues. As GRP receptors are well documented to be over-expressed in a number of cancer types such as prostate, breast and small cell lung cancer, a radiodiagnostic or radiotherapeutic agent that targets such receptors has the potential to be widely useful for the diagnosis or treatment of such cancers. The diagnostic application of the stabilized radiopharmaceuticals of the invention can be as a first line diagnostic screen for the presence of a disease state such as, for example, neoplastic cells using scintigraphic imaging, as an agent for targeting particular tissues (e.g., neoplastic tissue) using hand-held radiation detection instrumentation in the field of radio guided surgery (RIGS), as a means to obtain dosimetry data prior to administration of the matched pair radiotherapeutic compound, and as a means to assess, for example, receptor population as a function of treatment over time.

The therapeutic application of the stabilized radiopharmaceuticals of the invention can be as an agent that will be used as a monotherapy in the treatment of a disease, such as cancer, as combination therapy where these radiolabeled agents could be utilized in conjunction with adjuvant chemotherapy, and as the matched pair therapeutic agent. The matched pair concept refers to a single unlabeled compound which can serve as both a diagnostic and a therapeutic agent depending on the radioisotope that has been selected for binding to the appropriate chelate. If the chelator cannot accommodate the desired metals appropriate substitutions can be made to accommodate the different metal whilst maintaining the pharmacology such that the behaviour of the diagnostic compound in vivo can be used to predict the behaviour of the radiotherapeutic compound.

The stabilized compounds and formulations of the present invention can be administered to a patient alone or as part of a composition that contains other components such as excipients, diluents, and carriers, all of which are well-known in the art. The compounds can be administered to patients intravenously, subcutaneously, intra-arterially, intraperitoneally, intratumorally or by installation into resection cavities in, e.g., the brain. Stabilized radiolabeled scintigraphic imaging agents provided by the present invention are provided having a suitable amount of radioactivity. In forming ^{99m}Tc radioactive complexes, it is generally preferred to form radioactive complexes in solutions containing radioactivity at concentrations of from about 0.01 millicurie (mCi) to 100 mCi per mL. Generally, the unit dose to be administered has a radioactivity of about 0.01 mCi to about 100 mCi, preferably 1 mCi to 30 mCi. The solution to be injected at unit dosage is from about 0.01 mL to about 10 mL. For ¹¹¹In-labeled complexes, the unit dose to be administered typically ranges from about 0.01 mCi to about 10 mCi, preferably 3 to 6 mCi for diagnostic applications, and from 10 mCi to about 2 Curies for radiotherapeutic applications, preferably 30 mCi to 800 mCi. For ¹⁷⁷Lu-labeled complexes, the unit dose to be administered typically ranges from about 10 mCi to about 200 mCi, preferably from about 100 to about 200 mCi. The amount of labeled conjugate appropriate for administration is dependent upon the distribution profile of the chosen conjugate in the sense that a rapidly cleared conjugate may need to be administered in higher doses than one that clears less rapidly. In vivo distribution and localization can be tracked by standard scintigraphic techniques at an appropriate time subsequent to administration; typically between thirty minutes and 180 minutes depending upon the rate of accumulation at the target site with respect to the rate of clearance at non-target tissue. For example, after injection of the stabilized diagnostic radionuclide-labeled compounds of the invention into the patient, a gamma camera calibrated for the gamma ray energy of the nuclide incorporated in the imaging agent can be used to image areas of uptake of the agent and quantify the amount of radioactivity present in the site. Imaging of the site in vivo can take place in a few minutes. However, imaging can take place, if desired, hours or days after the radiolabeled compound is injected into a patient. In most instances, a sufficient amount of the administered dose will accumulate in the area to be imaged within about 0.1 hour to permit the taking of scintiphotos. With radiolabeled antibodies and antibody fragments, appropriate imaging times may be up to about one week following administration.

There are numerous advantages associated with the present invention. The compounds made in accordance with the present invention form stable, well-defined ¹¹¹In or ¹⁷⁷Lu labeled compounds. Similar stabilized compounds and formulations of the invention can also be made by using appropriate chelator frameworks for the respective radiometals, to form stable, well-defined products labeled with ¹⁵³Sm, ⁹⁰Y, ¹⁶⁶Ho, ¹⁰⁵Rh, ¹⁹⁹Au, ¹⁴⁹Pm, ^{99m}Tc, ^{186/188}Re or other radiometal. The stabilized radiolabeled GRP receptor targeting peptides selectively bind to neoplastic cells expressing GRP receptors, and if an agonist is used, become internalized, and are retained in the tumor cells for extended time periods. Because of the high radiostability obtained, the radioactive formulations do not undergo significant decomposition, and thus can be prepared at, for example, a central radiolabeling facility and then shipped to distant sites without significant decomposition and loss of targeting ability.

7. Radiotherapy

Radioisotope therapy involves the administration of a radiolabeled compound in sufficient quantity to damage or destroy the targeted tissue. After administration of the compound (by e.g., intravenous, subcutaneous, or intraperitonal injection), the stabilized radiolabeled pharmaceutical localizes preferentially at the disease site (e.g., tumor tissue that expresses a member of the GRP receptor family). Once localized, the radiolabeled compound then damages or destroys the diseased tissue with the energy that is released during the radioactive decay of the isotope that is administered.

The design of a successful radiotherapeutic involves several critical factors:

1. selection of an appropriate targeting group to deliver the radioactivity to the disease site;

2. selection of an appropriate radionuclide that releases sufficient energy to damage that disease site, without substantially damaging adjacent normal tissues; and

3. selection of an appropriate combination of the targeting group and the radionuclide without adversely affecting the ability of this conjugate to localize at the disease site. For radiometals, this often involves a chelating group that coordinates tightly to the radionuclide, combined with a linker that couples said chelate to the targeting group, and that affects the overall biodistribution of the compound to maximize uptake in target tissues and minimize uptake in normal, non-target organs.

4. Selection of appropriate radiostabilizers such that once formed, the radiotherapeutic compound does not undergo significant radiolytic decomposition prior to administration.

The present invention provides stabilized radiotherapeutic agents that satisfy all of the above criteria, through proper selection of stabilizer or stabilizers, targeting group, radionuclide, metal chelate [if present] and optional linker.

For radiotherapy applications any of the chelators for therapeutic radionuclides disclosed herein may be used. However, forms of the DOTA chelate [Tweedle M F, Gaughan G T, Hagan J T, “1-Substituted-1,4,7-triscarboxymethyl-1,4,7,10-tetraazacyclododecane and analogs.” U.S. Pat. No. 4,885,363, Dec. 5, 1989] are particularly preferred, as the DOTA chelate is expected to lose the bound radionuclide less in the body than DTPA or other linear chelates.

General methods for coupling DOTA-type macrocycles to targeting groups through a linker (e.g. by activation of one of the carboxylates of the DOTA to form an active ester, which is then reacted with an amino group on the linker to form a stable amide bond), are known to those skilled in the art. (See, e.g., Tweedle et al. U.S. Pat. No. 4,885,363; Current and potential therapeutic uses of lanthanide radioisotopes, Cutler, C, et al., Cancer Biotherapy & Radiopharmaceuticals (2000), 15(6), 531-545; Receptor targeting for tumor localisation and therapy with radiopeptides, Heppeler, A et al., Current Medicinal Chemistry (2000), 7(9), 971-994; Preparation methods for bifunctional chelatones for conjugation with antibodies, Budsky, F et al., Radioisotopy (1990), 31(4), 70-80)). Coupling can also be performed on DOTA-type macrocycles that are modified on the backbone of the polyaza ring.

The selection and amount of the proper stabilizer or stabilizer combination used to stabilize the radionuclide selected will also depend on the properties of the isotope selected, as, in general, nuclides that emit high energy alpha or beta radiation will have a requirement for more radiostabilizer than those that emit low energy radiation.

Many of the lanthanides and lanthanoids include radioisotopes that have nuclear properties that make them suitable for use as radiotherapeutic agents, as they emit beta particles. Some of these are listed in the table below.

| | | | | Approximate |
|---|---|---|---|---|
| | | | | range of b- |
| | | | | particle |
| | Half-Life | Max b- energy | Gamma energy | (cell |
| Isotope | (days) | (MeV) | (keV) | diameters |
| ¹⁴⁹-Pm | 2.21 | 1.1 | 286 | 60 |
| ¹⁵³-Sm | 1.93 | 0.69 | 103 | 30 |
| ¹⁶⁶-Dy | 3.40 | 0.40 | 82.5 | 15 |
| ¹⁶⁶-Ho | 1.12 | 1.8 | 80.6 | 117 |
| ¹⁷⁵-Yb | 4.19 | 0.47 | 396 | 17 |
| ¹⁷⁷-Lu | 6.71 | 0.50 | 208 | 20 |
| ⁹⁰-Y | 2.67 | 2.28 | — | 150 |
| ¹¹¹-In | 2.810 | Auger electron | 173, 247 | <5 μm |
| | | emitter | | |

Pm: Promethium,
Sm: Samarium,
Dy: Dysprosium,
Ho: Holmium,
Yb: Ytterbium,
Lu: Lutetium,
Y: Yttrium,
In: Indium

Methods for the preparation of radiometals such as beta-emitting lanthanide radioisotopes are known to those skilled in the art, and have been described elsewhere [e.g., Cutler C S, Smith C J, Ehrhardt G J.; Tyler T T, Jurisson S S, Deutsch E. “Current and potential therapeutic uses of lanthanide radioisotopes.” Cancer Biother. Radiopharm. 2000; 15(6): 531-545]. Many of these isotopes can be produced in high yield for relatively low cost, and many (e.g., ⁹⁰Y, ¹⁴⁹Pm, ¹⁷⁷Lu) can be produced at close to carrier-free specific activities (i.e., the vast majority of atoms are radioactive). Since non-radioactive atoms can compete with their radioactive analogs for binding to receptors on the target tissue, it is advantageous that isotopes that are essentially isotopically pure (i.e., free of their nonradioactive congeners) be used, to allow delivery of as high a dose of radioactivity to the target tissue as possible.

Stabilized radiotherapeutic derivatives of the invention containing beta-emitting isotopes of lutetium and yttrium (¹⁷⁷Lu and ⁹⁰Y) are particularly preferred.

8. Dosages and Additives

Proper dose schedules for the stabilized radiopharmaceutical compounds of the present invention are known to those skilled in the art. The stabilized compounds can be administered using many methods which include, but are not limited to, a single or multiple IV or IP injections, using a quantity of radioactivity that is sufficient to permit imaging or, in the case of radiotherapy, to cause damage or ablation of the targeted tissue, but not so much that substantive damage is caused to non-target (normal tissue). The quantity and dose required for scintigraphic imaging is discussed supra. The quantity and dose required for radiotherapy is also different for different constructs, depending on the energy and half-life of the isotope used, the degree of uptake and clearance of the agent from the body and the mass of the tumor. In general, doses can range from a single dose of about 30-200 mCi to a cumulative dose of up to about 3 Curies.

In addition to the stabilizers described in this application, the radiopharmaceutical compositions of the invention can include physiologically acceptable buffers, non-aqueous solvents, bulking agents and other lyophilization aids or solubilizing agents. They can be either in a liquid formulation [either frozen or at room temperature, or can be lyophilized (freeze dried).

A single, or multi-vial kit that contains all of the components needed to prepare the stabilized radiopharmaceuticals of this invention, other than the radionuclide, is an integral part of this invention.

In a preferred embodiment, a single-vial kit for the preparation of stabilized compounds preferably contains a chelator/optional linker/targeting peptide molecule, an optional source of stannous salt or other pharmaceutically acceptable reducing agent (if reduction is required, e.g., when using technetium or rhenium), and is appropriately buffered with pharmaceutically acceptable acid or base to adjust the pH to a value of about 3 to about 9. The quantity and type of reducing agent used will depend highly on the nature of the exchange complex to be formed. The proper conditions are well known to those that are skilled in the art. In one embodiment, the kit contents are in lyophilized form. Depending on the radioisotope used, such a single vial kit may optionally contain labile or exchange ligands such as acetate, glucoheptonate, gluconate, mannitol, malate, citric or tartaric acid and can also contain reaction modifiers such as diethylenetriamine-pentaacetic acid (DPTA), ethylenediamine tetraacetic acid (EDTA), or α, β, or γ-cyclodextrins and derivatives that serve to improve the radiochemical purity and stability of the final product. The kit may also contain bulking agents such as mannitol that are designed to aid in the freeze-drying process, and other additives known to those skilled in the art. The stabilizer or stabilizer combination selected should contain sufficient stabilizer to prevent significant decomposition of the product over the useful shelf-life of the reconstituted product.

A multi-vial kit preferably contains the same general components but employs more than one vial in reconstituting the radiopharmaceutical. For example, one vial may contain all of the ingredients that are required to form a labile Tc(V) or Re(V) complex on addition of pertechnetate (e.g., the stannous source or other reducing agent). Perteclmetate is added to this vial, and after waiting an appropriate period of time, the contents of this vial are added to a second vial that contains the chelator and targeting peptide, as well as buffers appropriate to adjust the pH to its optimal value and stabilizers sufficient to prevent radiolytic damage. After a reaction time of about 5 to 60 minutes, the complexes of the present invention are formed. It is advantageous that the contents of both vials of this multi-vial kit be lyophilized. As above, reaction modifiers, exchange ligands, stabilizers, bulking agents, etc. may be present in either or both vials.

9. Radiostabilizers

The presence of one or more radiostabilizers described herein is a requirement in stabilized formulations of the invention. The purpose of these stabilizers is to slow or prevent radiolytic damage to both the unlabeled and radiolabeled radiopharmaceuticals. Although some radiostabilizers are known, none of the literature has revealed the need for radiostabilizers for radiodiagnostic or radiotherapeutic GRP-receptor binding compounds. However, it has been found that stabilizers are required, especially as the amount of radioactivity in the formulation is increased, and when beta-emitting radiotherapeutic isotopes are used. As described by the examples below, many stabilizers have been identified that, alone or in combination, inhibit radiolytic damage to radiolabeled compounds. At this time, four approaches are the most preferred solutions to the problem.

In the first approach, a radiolysis stabilizing solution containing a mixture of the following ingredients is added to the radiolabeled compound immediately following the radiolabeling reaction: gentisic acid, ascorbic acid, human serum albumin, benzyl alcohol, a physiologically acceptable buffer or salt solution at a pH of about 4.5 to about 8.5, and in a preferred embodiment, one or more amino acids selected from methionine, selenomethionine, selenocysteine, or cysteine.

The physiologically acceptable buffer or salt solution is preferably selected from phosphate, citrate, or acetate buffers or physiologically acceptable sodium chloride solutions or a mixture thereof, at a molarity of from about 0.02M to about 0.2M.

In a preferred embodiment, the following concentrations are used: gentisic acid (2-20 mg/mL, most preferably about 10 mg/mL), ascorbic acid (10 to 100 mg/mL, most preferably about 50 mg/mL), human serum albumin (0.1 to 0.5%. most preferably about 0.2% (w/v)), benzyl alcohol (20 to 100 μL/mL, most preferably about 90 μL/mL), pH 4.5 to 8.0, most preferably about pH 5.0 citrate buffer (0.05 molar), and D- or L-methionine, L-selenomethionine, or L-cysteine (2 mg/mL).

Physiologically acceptable salts of the reagents may also be used (e.g. sodium ascorbate or sodium gentistate). D-, L-, and DL- forms of the amino acids may be used. Indeed, reference to a particular amino acid herein is intended to encompass use of the D-, L- and DL- forms of that amino acid.

The reagent benzyl alcohol is a key component in this formulation and serves two purposes. For compounds that have limited solubility, one of its purposes is to solubilize the radiodiagnostic or radiotherapeutic targeted compound in the reaction solution, without the need for added organic solvents. Its second purpose is to provide a bacteriostatic effect. This is important, as solutions that contain the radiostabilizers of the invention are expected to have long post-reconstitution stability, so the presence of a bacteriostat is desirable in order to maintain sterility. In a preferred embodiment, the amino acids methionine, selenomethionine, cysteine, and selenocysteine are also key components in this formulation and play a special role in preventing radiolytic damage to methionyl residues in targeted molecules that are stabilized with this radiostabilizing combination.

In the second approach, stabilization is achieved via the use of dithiocarbamate compounds having the following general formula: 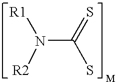
wherein R1 and R2 are each independently H; C1-C8 alkyl; —OR3, wherein R3 is C1-C8 alkyl; or benzyl (Bn) (either unsubstituted or optionally substituted with water solubilizing groups),
or wherein R1R2N combined are 1-pyrrolidinyl-, piperidino-, morpholino-, 1-piperazinyl- and M may be H⁺, Na⁺, K⁺, NH₄⁺, N-methylglucamine or other pharmaceutically acceptable +1 ion. Alternatively, compounds of the form shown below may be used, wherein M is a physiologically acceptable metal in the +2 oxidation state, such as Mg²⁺ or Ca²⁺, and R1 and R2 have the same definition as described above.

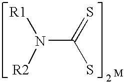

These reagents can either be added directly into reaction mixtures during radiolabeled complex preparation, or added after complexation is complete, or both.

The compound 1-Pyrrolidine Dithiocarbamic Acid Ammonium salt (PDTC) proved most efficacious as a stabilizer, when either added directly to the reaction mixture or added after complex formation. Use of this compound as a single reagent was effective at radioprotection of ¹⁷⁷Lu-A and ¹⁷⁷Lu-B (unlike in many of the studies above, where a combination of reagents had to be used). These results were unexpected, as the compound has not been reported for use as a stabilizer for radiopharmaceuticals prior to these studies. As shown in Example 20, dithiocarbamates such as PDTC provide the additional benefit of preventing contaminating metals from interfering with the labeling reaction.

In the third approach, formulations contain stabilizers that are water soluble organic selenium compounds wherein the selenium is in the oxidation state +2. Especially preferred are the amino acid compounds selenomethionine, and selenocysteine and their esters and amide derivatives and dipeptides and tri peptides thereof, which can either be added directly to the reaction mixture prior to or during radiolabeled complex preparation, or following complex preparation. The flexibility of having these stabilizers in the vial at the time of labeling or in a separ ate vial extends the utility of this invention for manufacturing radiodiagnostic or radiotherapeutic kits.

With these selenium compounds, it is highly efficacious to use these reagents in combination with sodium ascorbate or other pharmaceutically acceptable forms of ascorbic acid and its derivatives. The ascorbate is most preferably added after complexation is complete. Example 22 describes radiostabilization with this combination of reagents. Alternatively, it can be used as a component of the stabilizing formulation described above. If the selenium compound is an amino acid derivative such as selenomethionine or selenocysteine, then D-, L- and DL isomers of this amino acid derivative may be used.

A fourth approach involves the use of water soluble sulfur-containing compounds wherein the sulfur is in the +2 oxidation state. Preferred thiol compounds include derivatives of cysteine, mercaptothanol, and dithiolthreitol. These reagents are particularly preferred due to their ability to reduce oxidized forms of methionine residues (e.g., methionine oxide residues) back to methionyl residues, thus restoring oxidative damage that has occurred as a result of radiolysis. With these thiol compounds, it is highly efficacious to use these stabilizing reagents in combination with sodium ascorbate or other pharmaceutically acceptable forms of ascorbic acid and its derivatives. The ascorbate is most preferably added after complexation is complete. If the thiol compound is an amino acid derivative such as cysteine or cysteine ethyl ester, then D-, L- and DL isomers of this amino acid derivative may be used.

In the examples below, the use of stabilizing formulations containing examples of the four classes of reagents above are described. It should be understood that the four classes of agents can be used separately or in combination, as required to provide adequate radiostability to the radiodiagnostic or radiotherapeutic compound that is being stabilized. Although the examples provided focus primarily on the stabilization of compounds containing methionine which target the GRP receptor family, it is envisioned that this invention is much broader in scope. These methods of oxidative stabilization may be used to protect other radiodiagnostic or radiotherapeutics derived from, e.g., peptides, monoclonal antibodies, monoclonal antibody fragments, aptamers, oligonucleotides and small molecules, from oxidative degradation (not necessarily just methionine oxidation).

Potential stabilizers were evaluated for their ability to prevent or slow the decomposition of ¹⁷⁷Lu complexes of Compound A, referred to as ¹⁷⁷Lu-A, and ¹⁷⁷Lu complexes of Compound B, referred to as ¹⁷⁷Lu-B, their Indium-labeled analogs ¹¹¹In-A and ¹¹¹In-B, and other compounds in this class. Potential scavengers were evaluated in different ways: by either adding them directly to the reaction mixture used to form the ¹⁷⁷Lu or ¹¹¹In complexes, or by adding the stabilizer(s) after the radiometal complex was formed (or both). Several efficacious stabilizers and stabilizer combinations have been identified.

TABLE 1
Compounds tested as stabilizers and their structures

| | |
|---|---|
| 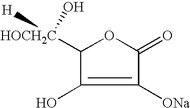 | Sodium L-ascorbate (Ascorbic acid) |
| 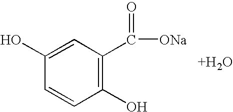 | 2,5-Dihydroxybenzoic acid sodium salt hydrate (Gentisic acid) |
| 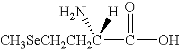 | L-Selenomethionine |
| 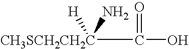 | D-Methionine (L and DL also used) |
| 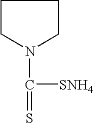 | 1-pyrrolidine dithiocarbamic acid ammonium salt |
| 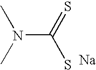 | Dimethyldithiocarbamate sodium salt |
| 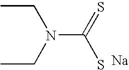 | Diethyldithiocarbamate sodium salt |
| 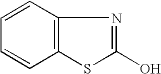 | 2-Hydroxybenzothiazole |
| 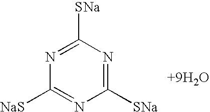 | Trithiocyanuric acid trisodium salt nonahydrate |
| 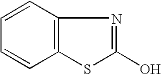 | 2-Hydroxybenzothiazole |
| 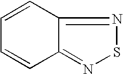 | 2,1,3-Benzothiadiazole |
| 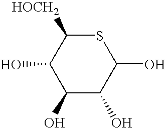 | 5-Thio-D-glucose |
| H₂NCH₂CH₂SSCH₂CH₂NH₂ +2HCl | Cystamine dihydrochloride |
| 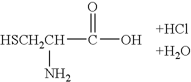 | L-cysteine hydrochloride monohydrate |
| 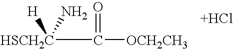 | L-cysteine ethyl ester hydrochloride |
| 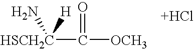 | L-cysteine methyl ester hydrochloride |
| 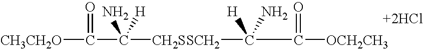 | L-cysteine diethyl ester dihydrochloride |
| 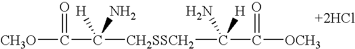 | L-cysteine dimethyl ester dihydrochloride |
| 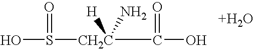 | L-cysteinesulfinic acid monohydrate |
| 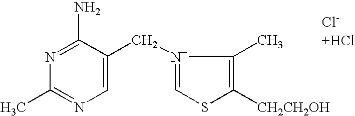 | Thiamine hydrochloride |
| 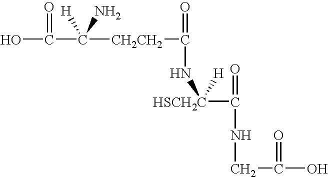 | L-Glutathione, reduced |
| 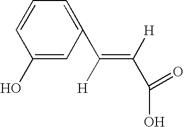 | 3-Hydroxycinnamic acid |
| 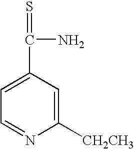 | 2-Ethyl-4-pyridinecarbothioamide (Ethionamide) |
| 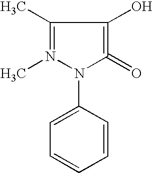 | 4-Hydroxyantipyrine |
| 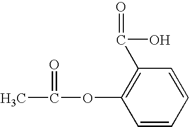 | Acetylsalicylic acid |
| 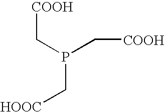 | Tris(carboxyethyl)phosphine |

Several studies were performed. The goal of these studies was to find stabilizer/targeted Lu-complex combinations that showed no significant detectable radio-degradation at a radioactivity concentration of >20 mCi/mL over time and in a preferred embodiment, to find stabilizers and stabilizer combinations able to provide five days of storage at room temperature (a reasonable period if the radiopharmaceutical has to be prepared and shipped) without significant detectable radio-degradation. Those that provided such stability were selected for further evaluation. Of the compounds tested, L-cysteine and the cysteine derivatives L-cysteine ethyl ester or L-cysteine methyl ester, D-, L-, and DL-methionine, L-selenomethionine, gentisic acid (Sodium salt), ascorbic acid (Sodium Salt) and 1-pyrrolidine dithiocarbamic acid ammonium salt (PDTC) were shown to be most efficacious in this respect when used as individual stabilizers.

In practice, a radiolysis protecting solution that contained a mixture of stabilizers proved especially useful. Formulations stabilized by such cocktails maintained excellent radiochemical purity (RCP) values (>95% RCP) for as long as 5 days at room temperature. This stabilizing cocktail is added immediately after formation of the radioactive complex, so would be the second vial of a two-vial kit. The reagents in this radiolysis protecting solution are shown in Table 2:

TABLE 2
Radiolysis Protecting Solution

| | Concentration | |
|---|---|---|
| | in Radiolysis | |
| | Protecting | |
| Reagent | Solution | |
| Gentisic acid | 10 | mg/mL |
| Ascorbic acid | 50 | mg/mL |
| Human serum albumin | 0.2% | (w/v) |
| Benzyl alcohol | 90 | μL/mL |
| pH 5.0 citrate buffer | 0.05 | molar |
| D- DL- or L-Methionine, L- | 2 | mg/mL |
| Selenomethionine, or L-cysteine | | |

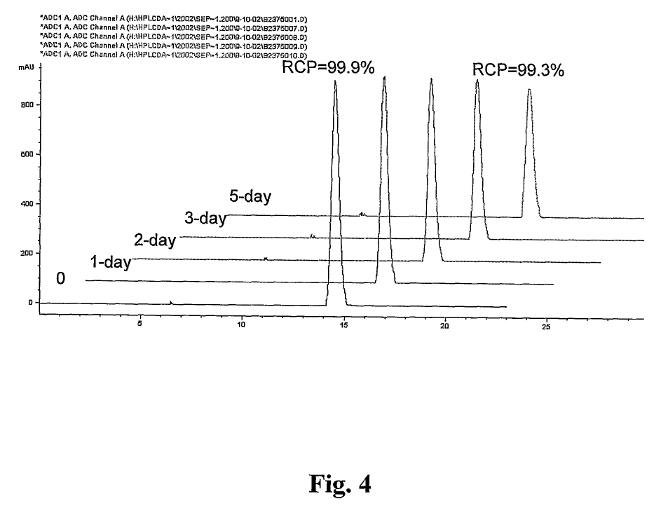

Stability in Radiolysis Protecting Solution: FIG. 4 shows the results obtained when 1 mL of a reaction mixture containing 104 mCi of ¹⁷⁷Lu-B was incubated at room temperature with 1 mL of the above radiolysis protecting solution that contained 2 mg/mL DL-methionine, 10 mg/mL gentisic acid, 50 mg/mL ascorbic acid, 0.2% HSA and 90 μl benzyl alcohol in 0.05 M citrate buffer, pH 5.3.

In a similar study, effective radiostabilization (RCP>95%) was achieved for ¹⁷⁷Lu-A if the concentration of methionine in the radiolysis protecting solution was increased to 3 mg/mL and all other reagents were held at their previous levels. ¹⁷⁷Lu-A was also stable for 5 days when methionine in the stabilizing cocktail is replaced by methionine, L-cysteine or L-selenomethionine.

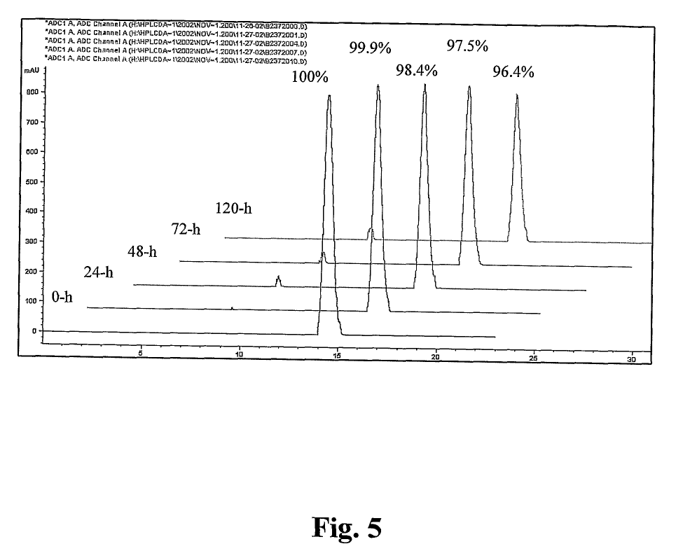

The data in FIG. 5 show the results obtained when 55 mCi of ¹⁷⁷Lu-A was incubated for 5 days at room temperature with the following mixture: 1.5 mg/mL L-cysteine; 5 mg/mL gentisic acid; 25 mg/mL ascorbic acid; 1 mg/mL HSA, 45 μL benzyl alcohol in 0.05M citrate buffer, pH 5.3.

Similar results to those found using L-cysteine could also be obtained using a radiolysis protecting solution containing L-selenomethionine or L- or D-methionine in the place of cysteine. Preliminary tolerance studies on stabilizing solutions containing these ingredients were performed in mice—no acute adverse effects were noted.

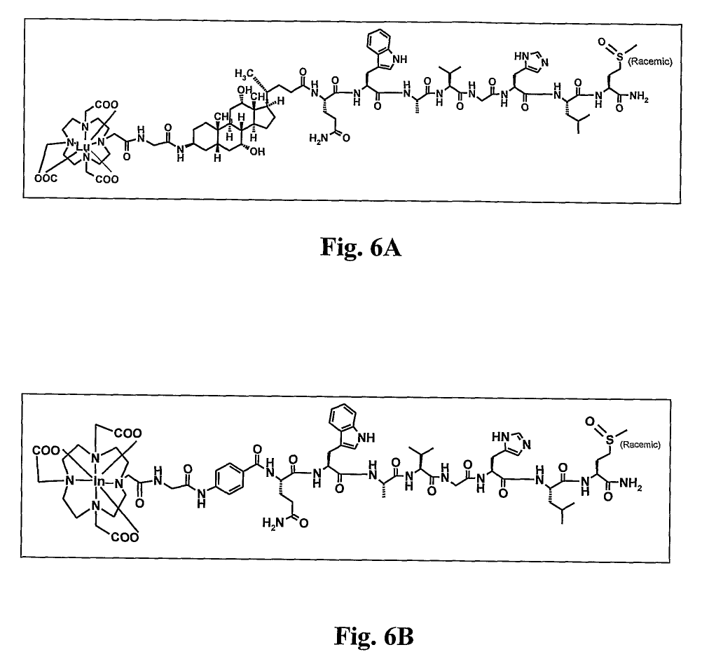

Role of the reagents in the radiolysis protecting solution: Studies have indicated that the methionine, L-selenomethionine, L-selenocysteine or L-cysteine in this stabilizing cocktail play a special role in the formulation, as these reagents appear to help prevent the oxidation of the methionine residue present in the GRP receptor-binding peptides to form analogs containing a methionine sulfoxide residue (see, e.g., FIG. 6A or FIG. 6B). As the oxidized methionine form of these peptides (Met=O derivative) is biologically inactive and has substantially reduced targeting ability, prevention of such oxidation is critical.

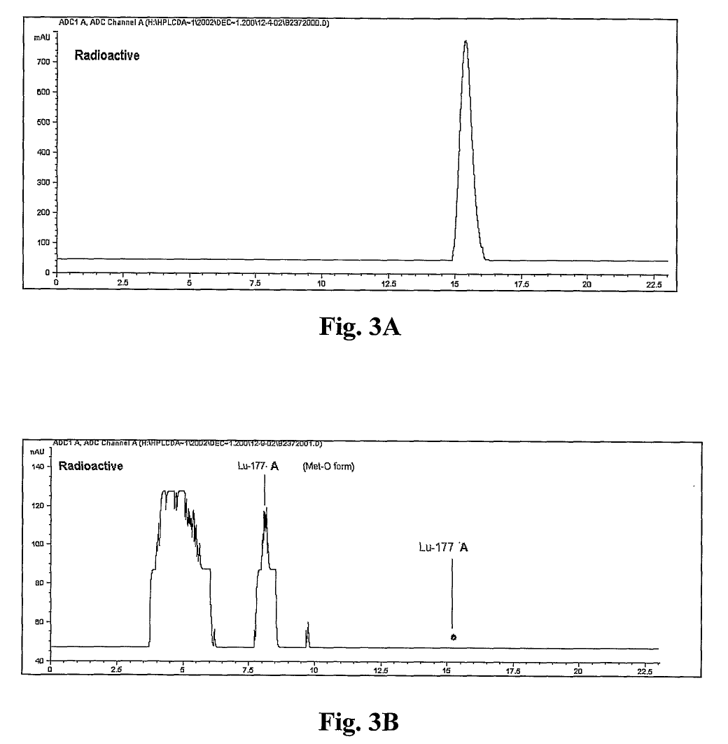

Methionine has been reported recently to be a stabilizer for radiodiagnostic compounds. However, in the present application (vide infra), it was determined that that methionine alone was insufficient to protect the compounds from radiolytic damage when high radioactivity levels are used, although some radiostabilization was observed (see, e.g., FIG. 3). However, the addition of the methionine-containing radiolysis protecting solution described above gives a strong protective effect that is not present when only methionine is used.

Organic compounds containing selenium in the +2 oxidation state: Organic compounds containing selenium in the +2 oxidation state, including selenomethionine and selenocysteine have not been reported as a radioprotectant for radiopharmaceuticals, nor has cysteine or other organic compounds containing thiols in the +2 oxidation state. Both of these compounds were found to be radioprotectants in their own right, and to have valuable properties if added to a radiolysis stabilizing solution as described in this disclosure.

Cysteine derivatives: L-cysteine, when added into a radiolysis stabilizing solution, appears to help prevent the oxidation of the methionine residue present in the GRP receptor-binding peptides. The ability of L-cysteine and of several cysteine derivatives (by themselves, rather than as part of a stabilizing cocktail) to effect such stabilization has been evaluated. All provide radioprotection to some extent, so the compounds cystamine dihydrochloride, L-cysteine hydrochloride monohydrate, L-cysteine ethyl ester hydrochloride, L-cysteine diethyl ester dihydrochloride, L-cysteine methyl ester hydrochloride, L-cysteine dimethyl ester dihydrochloride, L-cysteinesulfinic acid monohydrate are expected to have utility both as individual stabilizers and as components in stabilizing mixtures such as those described herein.

Likewise, it was determined that certain thiol-containing compounds, namely cysteine, 2-mercaptoethanol and dithiothreitol (DTT), can not only prevent radiolytically induced oxidation of the methionine residue present in GRP peptides, but can, in fact, reverse the process. As the oxidized methionine form of these peptides is biologically inactive, and has no targeting ability, this is a useful finding (that has not been described in the literature for the radioprotection of radiodiagnostics or radiotherapeutics). These reagents are also potential compounds in the stabilizing mixtures such as those described herein.

Dithiocarbamates: The examples provide evidence that dithiocarbamates, in particular the ammonium salt of 1-pyrrolidine dithiocarbamic acid, provide excellent stability as a single reagent without any additional stabilizers, when added to a radiolabeled peptide after complex formation (2-vial kit). 1-pyrrolidine dithiocarbamic acid (PDTC) and other dithiocarbamates have not been reported as radioprotectants for either radiodiagnostic or radiotherapeutic applications. The structure of PDTC is shown below.

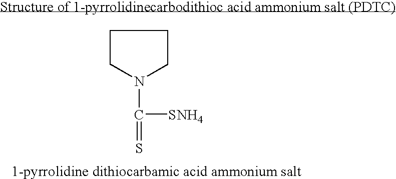

Two other dithiocarbamates, namely N,N-dimethyl dithiocarbamate and N,N-diethyl dithiocarbamate sodium salts were also evaluated and found to have a radiostabilizing effect, but the compound above was superior.

This compound is also extremely effective if added directly to the formulation during complex formation. At concentrations where it is an effective radiostabilizer, it does not interfere with complex formation. This is a clear advantage, as this allows a single-vial formulation, with all components in one vial.

Dithiocarbamates such as PDTC also have the added advantage of serving to scavenge adventitious trace metals in the reaction mixture. It has long been known that many radioisotopes (e.g., ⁹⁰Y, ¹¹¹In) can contain contaminating non-radioactive metals such as Fe, Zn, or Cu that can compete with the radiometal for the chelate. As the molar concentration of the radiometals used for radiotherapy is often very low, even a small amount of contaminating metal can be highly detrimental to a labeling reaction. This is especially true in formulations where the concentration of ligand has to be kept to a minimum in order to -obtain as high a specific activity [i.e., mCi of radioactivity/mmole of ligand] as possible.

If PDTC, for example, is added to reaction mixtures, it inhibits interference of adventitious metals, even if the contaminating metals are added in great excess. This result is surprising and unexpected.

It is expected that any compound of the general formula shown below will have potential utility. 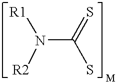
wherein R1 and R2 are each independently —H, —C1-C8 alkyl, —OR, phenyl, or benzyl (Bn) (either unsubstituted or optionally substituted with water solubilizing groups) or wherein R1R2N combined=1-pyrrolidinyl-, piperidino-, morpholino-, 1-piperazinyl- [optionally substituted with water solubilizing groups] and
M=H⁺, Na⁺, K⁺, NH₄⁺ or other pharmaceutically acceptable salt forms.

Preferred R1, R2 combinations are:

-Me, -Me;

-Me, -OMe;

-Et, -Et;

-Et, -OEt

-Et, -n-Bu;

-Me, —CH₂CH₂NMe₂;

-Me, —CH₂CH₂NMe₃⁺;

-Me, —CH₂COOMe),

-Bn, -Bn

It is expected that oxidized dimers of the compounds above [R1R2NC(S)S]₂ will be useful as well.

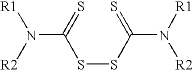

Use of the meglumine and glucamine compounds below is also envisioned. They have the advantage of being water soluble.

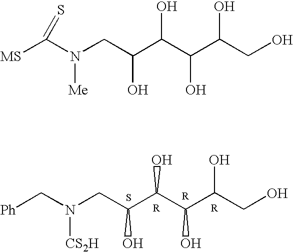

Alternatively, compounds of the form shown below may be used, wherein M is a physiologically acceptable metal in the +2 oxidation state, such as Mg²⁺ or Ca²⁺, and R1 and R2 have the same definition as described above.

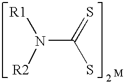

These reagents can either be added directly into reaction mixtures during radiolabeled complex preparation, or added after complexation is complete, or both.

The compound PDTC, and pharmacologically acceptable salts thereof, is particularly preferred.

Formulations with stabilizers added directly to reaction mixture: In most of the work described above, the stabilizer was added after formation of the radioactive complex. A series of studies were performed wherein different potential stabilizers were added directly to the reaction mixture during chelation. Such an approach is highly preferable, if a suitable compound can be found.

The following stabilizers were evaluated using this approach: 1-pyrrolidine dithiocarbamic acid ammonium salt, 2-bydroxybenzothiazole, 2,1,3-benzothiadiazole 5-thio-D-glucose, cystamine dihydrochloride, L-cysteine hydrochloride monohydrate, L-cysteine ethyl ester hydrochloride, L-cysteine diethyl ester dihydrochloride, L-cysteine methyl ester hydrochloride, L-cysteine dimethyl ester dihydrochloride, L-cysteinesulfinic acid monohydrate, sodium L-ascorbate (ascorbic acid), 2,5-dihydroxybenzoic acid sodium salt -hydrate (gentisic acid), thiamine hydrochloride, L-glutathione reduced, 2-ethyl-4-pyridinecarbothioamide (ethionamide), trithiocyanuric acid trisodium salt nonahydrate, sodium dimethyldithiocarbamate hydrate, sodium diethyldithiocarbamate trihydrate, 3-hydroxycinnamic acid, 4-hydroxyantipyrine and acetylsalicylic acid

It was found that the best stabilizers for direct addition to the formulation are the following: 1-pyrrolidine dithiocarbamic acid ammonium salt, D-, L-, or D,L-methionine, Trithiocyanuric acid trisodium salt, L-cysteine, or L-Selenomethionine. Of these, L-Selenomethionine and 1-pyrrolidine dithiocarbamic acid (ammonium salt) or pharmaceutically acceptable salts thereof are most preferred.

Since the stereochemistry of the amino acid is not critical to the stabilization the D-, L-, and D,L-mixtures of all amino acids previously cited are useful, as are pharmaceutically acceptable salts thereof. Simple derivatives of these amino acids including, but not limited to, N-alkylation, N-acetylation, C-terminus amidation or esterification are useful as well. It is anticipated that simple dipeptides, tripeptides, tetrapeptides and pentapeptides containing one or more of these amino acids could also be used to stabilize radiodiagnostic or radiotherapeutic formulations.

The following abbreviations are used in the description of the invention:

Acetonitrile (ACN)

Ethanol (EtOH)

Gentisic Acid (GA)

Glycine (Gly)

High Pressure Liquid Chromatography (HPLC)

Histidine (His)

Human Serum Albumin (HSA)

Hypophosphorous acid (HPA)

Indium (In)

Lutetium (Lu)

Mercaptoethanol (ME)

L- or D-Methionine (Met)

Phosphosaline buffer (PBS)

3,4-Pyridinedicarboxylic acid (Sodium salt) (PDCA)

1-pyrrolidine dithiocarbamic acid ammonium salt (PDTC)

Radiochemical purity (RCP)

L-Selenomethionine (Se-Met)

Technetium (Tc)

Trifluoroacetic acid (TFA)

Tris(carboxyethyl)phosphine (TCEP)

Trityl (Trt)

Tryptophan (Trp)

### EXAMPLES

Materials:

Trifluoroacetic acid (TFA), 1-pyrrolidine dithiocarbamic acid ammonium salt (PDTC), 2-hydroxybenzothiazole, 2,1,3-benzotbiadiazole, 5-thio-D-glucose, cystamine dihydrochloride, L-cysteine hydrochloride monohydrate, L-cysteine ethyl ester hydrochloride, L-cysteine dietbyl ester dihydrochloride, L-cysteine methyl ester hydrochloride, L-cysteine dimethyl ester dihydrochloride, L-cysteinesulfinic acid monohydrate, sodium L-ascorbate (ascorbic acid), 2,5-dihydroxybenzoic acid sodium salt hydrate (gentisic acid), thiamine hydrochloride, L-glutathione reduced, 2-ethyl-4-pyridinecarbothioamide (ethionamide), trithiocyanuric acid trisodium salt nonahydrate, sodium dimethyldithiocarbamate hydrate, sodium diethyldithiocarbamate trihydrate, 3-hydroxycinnamic acid, 4-hydroxyantipyrine and acetylsalicylic acid were purchased from Sigma-Aldrich Chemical Company. Acetic acid, glacial (ultra-pure) were purchased from J. T. Baker. Acetonitrile and sodium acetate, anhydrous (ultra-pure) was purchased from EM Science. D-methionine was purchased from Avocado Research Chemicals Ltd. L-selenometbionine was purchased from Calbiochem. Methanol, citric acid, anhydrous and sodium citrate were purchased from Fisher Scientific Company. Human serum albumin (HSA) was purchased from Sigma. All reagents were used as received. High-specific activity ¹⁷⁷LuCl₃ (in 0.05 N HCl) was obtained from the University of Missouri Research Reactor, Columbia, Mo. ¹¹¹InCl₃ (in 0.05N HCl) was obtained from either PerkinElmer or Mallinckrodt.

COMPOUND A (or Compound A) is the unmetallated ligand DOTA-Gly-ACA-Gln-Trp-Ala-Val-Gly-His-Leu-Met-NH₂ (ACA=3-Amino-3-deoxycholic acid). COMPOUND B (or Compound B) is the unmetallated ligand DOTA-Gly-Abz4-Gln-Trp-Ala-Val-Gly-His-Leu-Met-NH₂ (Abz4=4-aminobenzoic acid. The radiolabeled complexes prepared from these compounds are designated herein by the isotope-compound letter, i.e., ¹⁷⁷Lu-A is the ¹⁷⁷Lu complex of DOTA-Gly-ACA-Gln-Trp-Ala-Val-Gly-His-Leu-Met-NH₂) and ¹⁷⁷Lu-B is the ¹⁷⁷Lu complex of DOTA-Gly-Abz4-Gln-Trp-Ala-Val-Gly-His-Leu-Met-NH₂. The synthesis of Compounds A and B is described in applicants' copending patent application Ser. No. 10/341,577, filed Jan. 13, 2003, which is hereby entirely incorporated by reference.

Analytical Methods:

HPLC Method 1 used an HP-1100 HPLC system (Agilent) with a variable wavelength detector (?=280 nm) and a Canberra radio-detector, a YMC Basic S-5 column (4.6 mm×150 mm, 5 μm) and mobile phases A: Sodium citrate in water (0.02 M, pH 3.0), and B: 20% methanol in acetonitrile. The mobile phase flow rate was 1 mL/min. with a gradient starting at 32% B to 34% B over 30 minutes, 34% to 40% B in 5 minutes, back to 32% B in 5 minutes, then a 5-minute hold for re-equilibration. The injection volume was 20 μL.

HPLC Method 2 involved the use of an HP-1100 HPLC system with a variable wavelength detector (?=280 nm) and a Canberra radio-detector, a YMC Basic S-5 column (4.6 mm×150 mm, 5 μm) and mobile phases A: 0.1% TFA and 0.1% acetonitrile in water, and B: 0.1% TFA in acetonitrile. The mobile phase flow rate was 1 mL/min with a gradient starting at 29% B to 32% B over 20 minutes, back to 29% B in 2 minutes, then a 5-minute hold for re-equilibration. The injection volume was 20 μL.

HPLC Method 3 involved the use of an HP-1100 HPLC system with a variable wavelength detector (?=280 nm) and a Canberra radio-detector, a C18 column (4.6 mm×250 mm, 5 μm, VYDAC, cat#218TP54) and mobile phases A: 0.1% TFA in water, and B: 0.1% TFA in acetonitrile. The mobile phase flow rate was 1 mL/min with a gradient starting at 29% B to 32% B over 20 minutes, back to 29% B in 3 minutes, then an 8-minute hold for re-equilibration. The injection volume was 20 μL.

HPLC Method 4 involved the use of an HP1100 HPLC system with a variable wavelength detector (?=280 nm) and a Canberra radio-detector, a C18 column (4.6 mm×250 mm, 5 μm, VYDAC, Cat#218TP54) and mobile phases A: 0.1% TFA in water, and B: 0.1% TFA in acetonitrile. The mobile phase flow rate was 1 mL/min. with a gradient starting at 21% B to 24% B over 20 minutes, back to 21% B in 3 minutes, then an 8 minute hold for re-equilibration. The injection volume was 20 μL.

HPLC Method 5 involved the use of an HP-1100 HPLC system with a variable wavelength detector (?=280 nm) and a Canberra radio-detector, a Stellar Phases Rigel C18 column (4.6 mm×150 mm, 5 μm) and mobile phases A: 0.1% TFA and 0.1% ACN in water, and B: 0.1% TFA in ACN. The mobile phase flow rate was 1 mL/min. with a gradient starting at 20% B, ramping to 24% B over 20 minutes, back to 20% B in 2 minutes, then a 3 minute hold for re-equilibration. The injection volume was 10 μL.

### Example 1

### Comparison of the Radioprotective Effects of Various Amino Acids When Added to Pre-Formed ¹⁷⁷Lu-GRP Binding Compounds ¹⁷⁷Lu-A or ¹⁷⁷Lu-B

EXAMPLE 1 shows the results obtained for a series of amino acids that were added individually to a solution of ¹⁷⁷Lu-A or ¹⁷⁷Lu-B and then incubated at room temperature over 48 hours, as well as results for an unstabilized control. In these reactions, the amino acid concentration was 6.6 mg/mL, ¹⁷⁷Lu-A and ¹⁷⁷Lu-B had a concentration of ˜20 mCi/mL, and 3.5 mCi of ¹⁷⁷Lu was used in each reaction.

Solutions of the individual amino acids L-Methionine, L-Selenomethionine, L-cysteine HCl.H₂O, L-Tryptophan, L-Histidine, and Glycine were prepared at a concentration of 10 mg/mL in 10 mM Dulbecco's phosphate-buffered saline, pH 7.0 [PBS].

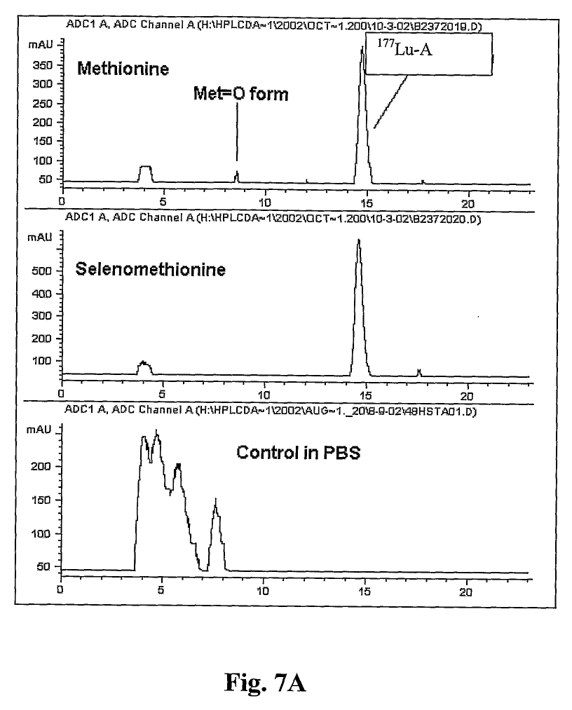

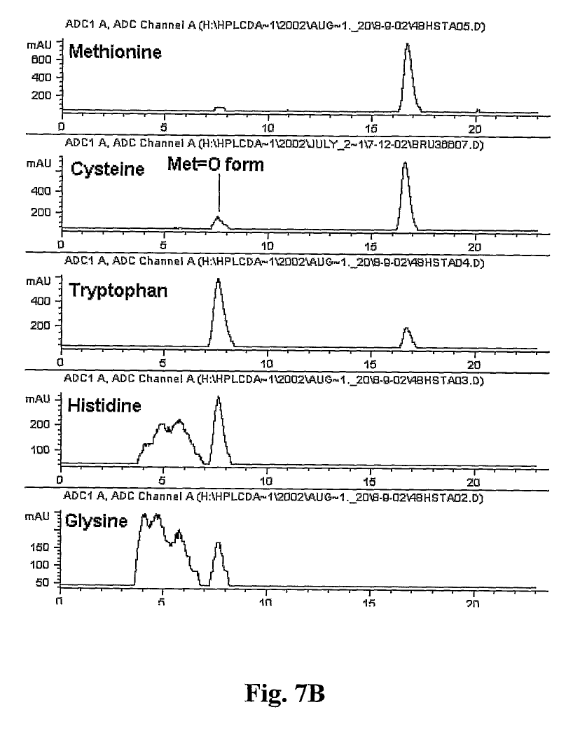

¹⁷⁷Lu-A and ¹⁷⁷Lu-B were prepared by adding 300 μL of 0.2 M NaOAc (pH 5.0), 40 μg Compound A or B and 20 mCi of ¹⁷⁷LuCl₃ into a reaction vial. The mixture was incubated at 100° C. for five minutes, then cooled to room temperature. Free (uncomplexed) ¹⁷⁷Lu in the reaction solution was then scavenged (chelated) by adding 10 μL of a 10% Na₂EDTA.2H₂O solution in water. A 50 μL aliquot of the reaction solution (˜3.5 mCi) was mixed with 100 μL of one of the amino acid solutions above or a PBS control in a 2-mL autosampler vial. The final radioactive concentration of each sample was 20 mCi/mL. The samples were stored in the autosampler chamber, and their stability over 48 hours was analyzed using HPLC Method 3 (¹⁷⁷Lu-A) or HPLC Method 4 (¹⁷⁷Lu-B). Chromatograms from this study at the 48-hour time point are shown in FIG. 7.

In the control reaction with no stabilizer, radiochemical purity (RCP) dropped from >95% to 1.3% within 24 hrs at room temperature. In contrast, when methionine, L-selenomethionine or cysteine was added, RCP remained greater than 90% for 48 hours.

Table 3 below shows the RCP values obtained in this study for all samples of ¹⁷⁷Lu-A at t=0, 24, and 48 hours.

TABLE 3
Evaluation of amino acids as radioprotectants for ¹⁷⁷Lu-A. Stability
comparison made by adding different individual amino acids (6.6 mg/mL)
to ¹⁷⁷Lu-A at a radioactive concentration of ˜20 mCi/mL, followed
by storage at room temperature for up to 48 hours. (3.5 mCi total)

| | 0-1 h | | 24-h | | 48-h | |
|---|---|---|---|---|---|---|
| Stabilizer | Met = O | RCP | Met = O | RCP | Met = O | RCP |
| added | (%) | (%) | (%) | (%) | (%) | (%) |
| Methionine | 0 | 100 | 0.7 | 99.3 | 5.1 | 94 |
| Se-Met | 0 | 100 | 0.9 | 99.1 | 0.1 | 99.9 |
| Cysteine | 0 | 100 | 1.2 | 98.8 | 5.7 | 94.3 |
| Tryptophan | 4.5 | 95.5 | 46.8 | 51.4 | 81.8 | 18.2 |
| Glycine | 3.6 | 96.4 | 24.2 | 14.6 | 13.8 | 0 |
| Histidine | 7.5 | 92.5 | 44 | 4.6 | 29.5 | 0 |
| Control(PBS) | 4.5 | 76.5 | 1.8 | 1.3 | 0 | 0 |
| * Only RCP and percentage of the methionine oxidized (Met = O) form of ¹⁷⁷Lu-A are listed; the remaining activity is in the form of unidentified degradants. These results demonstrate that the amino acids tested varied widely in their ability to stabilize ¹⁷⁷Lu-A and ¹⁷⁷Lu-B. Of the amino acids tested in this study, methionine, L-selenomethionine or L-cysteine provided the highest degree of protection | | | | | | |
| # against radiolytic decomposition of the ¹⁷⁷Lu-labeled peptides. In this study, it was found that tryptophan, a compound previously reported to be an effective stabilizer surprisingly did not protect against oxidation of the methionine residue present in the targeting peptides, although cysteine, methionine and selenomethionine were effective. | | | | | | |

### Example 2

### Further Evaluation of the Radioprotective Effect of L-Methionine for Radioprotection of ¹⁷⁷Lu-A (50 mCi/2mL)

Based on the results seen in EXAMPLE 1, the ability of L-methionine to protect ¹⁷⁷Lu-A when added after complex formation was studied. In contrast to EXAMPLE 1 above, in this reaction, 50 mCi of ¹⁷⁷Lu-A was used, rather than 3.5 mCi.

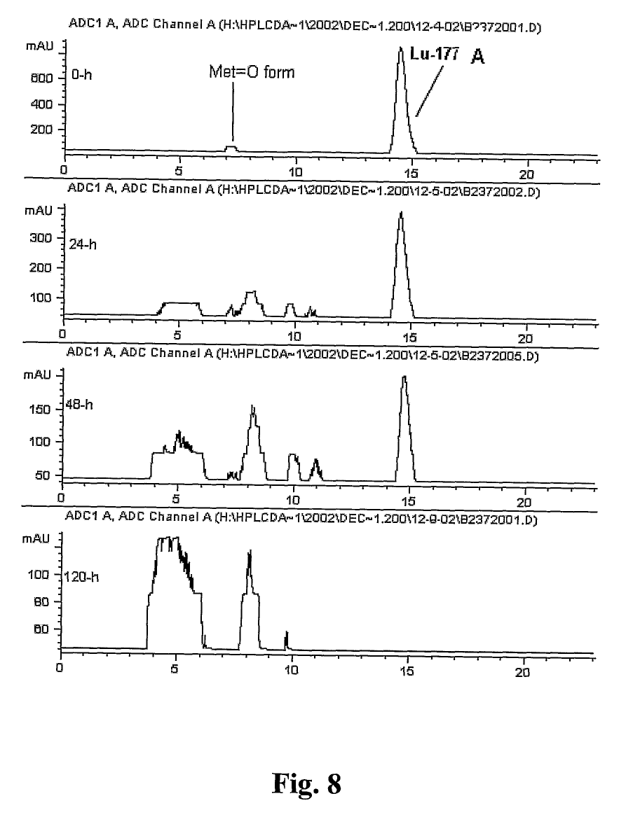

¹⁷⁷Lu-A was formed by adding ˜70 μg of Compound A and 50 mCi of ¹⁷⁷LuCl₃ (molar ratio of peptide to Lutetium of 3:1) to 1 mL of 0.2M NaOAc, pH 5.0. The mixture was heated at 100° C. for 5 min, cooled to room temperature in a water bath, and 1 mL of a 5 mg/mL L-methionine solution in water and 1 mg Na₂EDTA.2H₂O was added into the reaction vial. The chromatograms in FIG. 8 and the data in Table 4 below demonstrate the changes in radiochemical purity observed over 5 days at room temperature, when analyzed by reversed phase HPLC using HPLC Method 3. Table 4 summarizes the results shown in FIG. 8.

TABLE 4
¹⁷⁷Lu-A (50 mCi in 2 mL) stabilized by the
addition of 2.5 mg/mL L-methionine [Met] over 5 days
incubation at room temperature (% RCP):

| | % RCP t = 0 to 5 days | | | |
|---|---|---|---|---|
| Stabilizer | 0-d | 1-d | 2-d | 5-d |
| 5 mg Met | 96.1 | 53.5 | 26.9 | 0 |

In EXAMPLE 1, methionine at a concentration of 2.5 mg/mL was able to stabilize 3.5 mCi of ¹⁷⁷Lu-A against radiolysis for 5 days. However, the results seen in EXAMPLE 2 show that methionine is unable to stabilize the same complex when the amount of radioactivity is increased to 50 mCi. Almost complete decomposition of the complex was observed over 5 days, when only L-methionine was used as a stabilizer. As current practice dictates the use of 100 mCi or more of a radiolabeled peptide for radiotherapeutic applications, it is clear that a more efficacious stabilizer or stabilizer combination is required.

Similar studies were performed with L-cysteine, selenomethionine, sodium ascorbate, gentisic acid and HSA. None of them provided sufficient stabilization to use alone with the high radioactivity levels tested.

### Example 3

### Evaluation of the Radioprotective Effect of Various Reagents When Added to Pre-Formed ¹⁷⁷Lu-A (3.5 mCi)

The list of the potential radiolysis protecting agents tested in this experiment is as follows:

1. Ascorbic acid (Sodium salt form)

2. Gentisic acid (Sodium salt form)

3. Human Serum Albumin (HSA)

4. 3,4-pyridinedicarboxylic acid (Sodium salt) (PDCA)

5. 10% Ethanol aqueous solution

6. 2% Hypophosphorous acid (HPA)

7. 2% Mercaptoethanol (ME)

8. Tris(carboxyethyl)phosphine (TCEP)

9. Control (Phosphosaline buffer, pH 7.0)

Reagents 1-5 have been reported previously to be potentially useful as stabilizers for radiopharmaceuticals. Reagents 6-8 are compounds that were tested to determine their ability to serve as reducing agents for any methionine sulfoxide residues that formed as a result of radiolysis. Reagent 9 was used in the unstabilized control.

¹⁷⁷Lu-A was prepared by adding 300 μL of 0.2 M NaOAc (pH 5.0), 40 μg Compound A and 20 mCi of ¹⁷⁷LuCl₃ into a reaction vial. The mixture was incubated at 100° C. for five minutes, and then cooled to room temperature. Free ¹⁷⁷Lu was scavenged by adding 10 μL of 10% Na₂EDTA.2H₂O. A 50 μL aliquot of the reaction solution (˜3.5 mCi) and 100 μL of a 10 mg/mL solution of one of the reagents above in 10 mM, pH 7.0 PBS was added into a 2-mL autosampler vial. Alternatively, for reagents 5-7, the solution was adjusted to contain 10% Ethanol, 2% Hypophosphorous acid, or 2% Mercaptoethanol. The final radioactivity concentration was about 20 mCi/mL. The samples were stored in the autosampler chamber, and their stability was analyzed over time. The results obtained are shown in Table 5 below.

TABLE 5
Stability of ¹⁷⁷Lu-A at a radioactivity concentration of ˜20
mCi/mL, when incubated at room temperature over time with potential
non-amino acid radiolysis protecting agents at a concentration
of 6.6 mg/mL, or as otherwise mentioned*.

| | 0-h | | 24-h | | 48-h | |
|---|---|---|---|---|---|---|
| | Met = O | RCP | Met = O | RCP | Met = O | RCP |
| | (%) | (%) | (%) | (%) | (%) | (%) |
| Ascorbic acid | 2.5 | 97.5 | 11.8 | 72.1 | 14.2 | 24.9 |
| Gentisic acid | 2.4 | 97.6 | 9.2 | 90.8 | 17.2 | 82.8 |
| HSA | 4.4 | 95.6 | 13 | 20 | 6.2 | 2.5 |
| PDCA | 3.4 | 86.6 | 5 | 14.1 | N/A | N/A |
| 10% Ethanol | 1.4 | 98.6 | 7.2 | 92.8 | 13.5 | 80.6 |
| TCEP** | 7.2 | 96.1 | 0 | 23.4 | 0 | 6.2 |
| % HPA*** | 0 | 0 | 0 | 0 | 0 | 0 |
| 2% ME | 0.1 | 91.5 | 9.3 | 81.8 | 13.8 | 76.2 |
| Control (PBS) | 2.5 | 92.5 | 0 | 0 | 0 | 0 |

*Ethanol, Hypophosphorous acid (HPA) and Mercaptoethanol (ME) are in liquid form.
**TCEP = tris carboxyethyl phosphine
** 2% Hypophosphorous acid solution was prepared in 0.1 M, pH 7.8 phosphorous buffer to get a final pH of 5.5.
PBS = Phosphosaline buffer, pH 7.0

Table 5 above shows the results of a comparative study to determine the radiostabilizing effect of several compounds when added to ¹⁷⁷Lu-A after complex formation. Both the ability of these additives to prevent a decrease in RCP and their ability to inhibit the oxidation of the Methionine residue in ¹⁷⁷Lu-A were studied.

It was found that under the test conditions used, none of the eight reagents tested [Ascorbic acid (Sodium salt), gentisic acid (Sodium salt), Human Serum Albumin (HSA), Tris(carboxyethyl)phosphine (TCEP), 3,4-pyridinedicarboxylic acid (Sodium salt) (PDCA), 2% hypophosphorous acid (HPA), 2% mercaptoethanol (ME), or 10% ethanol aqueous solution] provided adequate radiostability (RCP>90%) for 48 hours. This result was unexpected, as gentisic acid, ascorbic acid, HSA and 3,4-pyridinedicarboxylic acid have all been reported by others to provide satisfactory protection against radiolysis for other radiopharmaceuticals. Although some radioprotection was observed when compared to the control in PBS, the previously reported stabilizers ascorbic acid, gentisic acid, and HSA were insufficient to maintain 48 hour stability at an RCP value greater than 90%. The reagent 3,4-pyridinedicarboxylic acid, previously reported as an effective radiostabilizer, was found to interfere badly with the labeling reaction. Mercaptoethanol and ethanol provided some degree of radiostabilization, but again, RCP values of <90% were found after 48 hours. TCEP and HPA were ineffective under the conditions used.

### Example 4

### Effect of Methionine-Containing Radiolysis Protecting Solution on RCP of ¹⁷⁷Lu-A and ¹⁷⁷Lu-B (50 mCi)

In the studies described in EXAMPLES 1-3, it was found that no single reagent tested was entirely effective as a radioprotectant that could provide protection from radiolytic decomposition of ¹⁷⁷Lu-GRP binding peptides at high radioactivity levels, especially with respect to oxidation of the terminal Methionine residue.

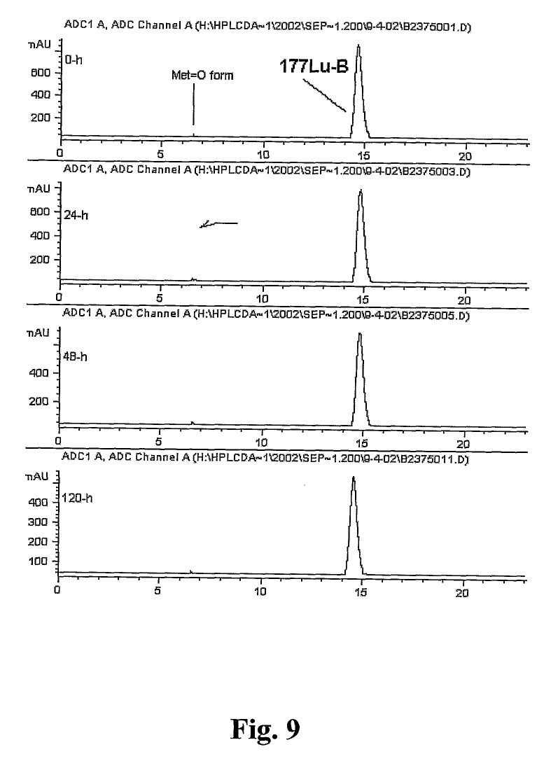

A Radiolysis Protecting Solution was prepared to contain 10 mg/mL gentisic acid; 50 mg/mL ascorbic acid sodium salt; 2 mg/mL HSA; 2.98 mg/mL L-methionine, 0.9% (v:v) benzyl alcohol and 1 mg/mL of Na₂EDTA.2H₂O in 0.05 M, pH 5.3 citrate buffer. To a 7-mL vial were added 0.2M NaOAc buffer (1.0 mL, pH 5.0), Compound A or Compound B (˜70 μg) and 50 mCi of ¹⁷⁷LuCl₃. The mixture was incubated at 100° C. for 5 min, and then cooled to room temperature with a water bath. A 1-mL aliquot of the Radiolysis Protecting Solution was immediately added. The reaction vial was stored in an autosampler chamber and the stability was analyzed by Reversed Phase HPLC over time, using HPLC Methods 3 and 4. The results obtained for ¹⁷⁷Lu-B are shown in the chromatograms in FIG. 9.

Similar results were obtained for ¹⁷⁷Lu-A (see Table 6 below).

TABLE 6
Stability comparison of ¹⁷⁷Lu-A or ¹⁷⁷Lu-B (50 mCi/2 mL)
in a Radiolysis Protecting Solution containing L-methionine over
5 days incubation at room temperature (% RCP)

| | % RCP | | | |
|---|---|---|---|---|
| | 0-d | 1-d | 2-d | 5-d |
| ¹⁷⁷Lu-A | 100 | 100 | 100 | 100 |
| ¹⁷⁷Lu-B | 99.8 | 99.3 | 99.6 | 99.8 |

These results demonstrate that when a radiolysis protecting solution containing gentisic acid, ascorbic acid, benzyl alcohol, methionine and HSA in citrate buffer is added to ¹⁷⁷Lu-A or ¹⁷⁷Lu-B, excellent radiostability is obtained, as indicated by no significant drop in the RCP over five days. This result was unexpected, as none of the reagents on their own were capable of providing stability for at least 5 days at room temperature, as indicated by a radiochemical purity of >99% after 120 hours. The radiostability provided by the methionine-containing Radiolysis Protecting Solutions would not have been predicted based on the efficacy of the individual reagents.

### Example 5

### Effect of L-Selenomethionine-Containing Radiolysis Protecting Solution on RCP of ¹⁷⁷Lu-A and ¹⁷⁷Lu-B (50 mCi/2 mL)

¹⁷⁷Lu-A and ¹⁷⁷Lu-B were prepared at a 50 mCi level as described in EXAMPLE 4. Immediately after cooling the reaction mixtures to room temperature, 1 mL of a Radiolysis Protecting solution was added, containing 10 mg/mL gentisic acid; 50 mg/mL ascorbic acid sodium salt; 2 mg/mL HSA; 3.92 mg/mL L-selenomethionine, 0.9% (v:v) benzyl alcohol and 1 mg/mL of Na₂EDTA.2H₂O in 0.05 M, pH 5.3 citrate buffer. The reaction vials were stored in the autosampler chamber and the stability was analyzed by RP-HPLC over time using HPLC Methods 3 [¹⁷⁷Lu-A] or 4 [¹⁷⁷Lu-B]. The results are shown in Table 7 below.

TABLE 7
Stability of ¹⁷⁷Lu-A or ¹⁷⁷Lu-B in Radiolysis Protecting
Solution containing L-selenomethionine over 5 days
incubation at room temperature (% RCP).

% RCP

| Complex | 0-d | 1-d | 2-d | 3-d | 5-d |
|---|---|---|---|---|---|
| ¹⁷⁷Lu-A | 97.6 | 98.2 | 97.5 | 97.8 | 99.4 |
| ¹⁷⁷Lu-B | 95.8 | 95.7 | 96.2 | 96.7 | 98.4 |

These results were unexpected, as none of the reagents on their own were capable of providing stability for at least 5 days at room temperature, as indicated by a radiochemical purity of >98% after 120 hours. The radiostability provided by the selenomethionine-containing Radiolysis Protecting Solutions would not have been predicted based on the efficacy of the individual reagents.

### Example 6

### Effect of L-Cysteine-Containing Radiolysis Protecting Solution on RCP of ¹⁷⁷Lu-A and ¹⁷⁷Lu-B (50 mCi/2 mL)

¹⁷⁷Lu-A and ¹⁷⁷Lu-B were prepared at a 50 mCi level as described in EXAMPLE 4. Immediately after cooling the reaction mixtures to room temperature, 1 mL of a Radiolysis Protecting Solution was added, containing 10 mg/mL gentisic Acid; 50 mg/mL ascorbic acid sodium salt, 2 mg/mL HSA, (2 mg/mL or 3.52 mg/mL) L-cysteine, 0.9% (v/v) benzyl alcohol and 1 mg/mL of Na₂EDTA.2H₂O in 0.05 M, pH 5.3 citrate buffer. The reaction vials were stored in the autosampler chamber and the stability was analyzed by RP-HPLC over time using HPLC Methods 3 [¹⁷⁷Lu-A] or 4 [¹⁷⁷Lu-B]. The results obtained for ¹⁷⁷Lu-A are shown in Table 8 below. Similar results were obtained for ¹⁷⁷Lu-B.

TABLE 8
Stability of ¹⁷⁷Lu-A (50 mCi/2 mL) in Radiolysis Protecting
Solution containing L-cysteine at 1.0 or 1.75 mg/mL over
5 days incubation at room temperature (% RCP)

| Concentration | |
|---|---|
| of L-cysteine | % RCP |

| (mg/mL) | 0-d | 1-d | 2-d | 3-d | 5-d |
|---|---|---|---|---|---|
| 1.0 | 100 | 99.9 | 98.4 | 97.5 | 96.9 |
| 1.75 | 100 | 99.9 | 98.9 | 95.8 | 93.3 |

These results were unexpected, as none of the reagents on their own were capable of providing stability for at least 5 days at room temperature, as indicated by a radiochemical purity of >93% after 120 hours. The radiostability provided by the cysteine-containing Radiolysis Protecting Solutions would not have been predicted based on the efficacy of the individual reagents.

### Example 7

### Effect of Radiolysis Protecting Solution on RCP of ¹⁷⁷Lu-A (50 mCi/2 mL)

¹⁷⁷Lu-A was prepared at a 50 mCi level as described in EXAMPLE 4. Immediately after cooling the reaction mixture to room temperature, 1 mL of a Radiolysis Protecting Solution was added, that contained 10 mg/mL gentisic acid; 50 mg/mL ascorbic acid sodium salt; 2 mg/mL HSA; 0.9% (v:v) benzyl alcohol and I mg/mL of Na₂EDTA.2H₂O in 0.05 M, pH 5.3 citrate buffer. The reaction vial was stored in an autosampler chamber and the stability was analyzed by RP-HPLC over time. The results are shown in Table 9 below.

TABLE 9
Stability of ¹⁷⁷Lu-A (50 mCi/2 mL) in a Radiolysis Protecting
Solution over 5 days incubation at room temperature (% RCP)

Concentration

| of L-cysteine | % RCP | | | | |
|---|---|---|---|---|---|
| (mg/mL) | 0-d | 1-d | 2-d | 3-d | 5-d |
| 0 | 100/100 | 94.9/99.2 | 93.6/97.3 | ND/95.1 | 90.4/92.9 |

ND = not determined

The results shown in EXAMPLES 4-7 demonstrate that addition of methionine (Example 4), selenomethionine (Example 5) or cysteine (Example 6) to the Radiolysis Protecting Solution described in EXAMPLE 7 provides added benefit beyond that of Radiolysis Protecting Solution prepared without these added amino acids.

### Example 8

### Effect of HSA or AA on the Radiostability of ¹⁷⁷Lu-B When Added After Radiolabeling

In this example, the effect of two reagents in the Radiolysis Stabilizing Solution, HSA and ascorbic acid; both known for their radioprotecting ability, were tested individually at very high concentrations (50-100 mg/mL). As individual reagents, they were again found insufficient to maintain ¹⁷⁷Lu-B at RCP values >95% for more than 24 hours. ¹⁷⁷Lu-B was formulated as follows: To a 5-mL glass vial, 1 mL of 0.2 M NaOAc buffer (pH 4.8), 12 μL (50 mCi) of ¹⁷⁷LuCl₃ and 30 μL of a 5 mg/mL solution of COMPOUND B in 0.01N HCl were added, and the vial was heated at 100° C. for 5 min. After being cooled in a water bath, the reaction mixture was diluted 1:1 by addition of 1 mL of one of the stabilizing solutions below. The samples were then stored in an autosampler (which maintained an average temperature that was ˜6° C. higher than room temperature) and analyzed by RP-HPLC for up to 120 hours.

Studies with HSA and Ascorbic Acid: In this study, three different stabilizing solutions (a, b, or c) were evaluated and compared.

a) Human Serum Albumin (HSA) was dissolved to a concentration of 100 mg/mL in N₂-purged 0.05 M, pH 5.0 citrate buffer containing 1 mg/mL Na₂EDTA.2H₂O

b) Sodium ascorbate (AA) 99+% was dissolved to a concentration of 100 mg/mL in N₂-purged 0.05 M, pH 5.0 citrate buffer containing 1 mg/mL Na₂EDTA.2H₂O

c) Sodium Ascorbate 99+% was dissolved to concentration of 50 mg/mL in N₂-purged 0.05 M, pH 5.0 citrate buffer containing 0.9% Benzyl alcohol and 1 mg/mL Na₂EDTA.2H₂O

The RCP results obtained are shown in Table 10.

TABLE 10
Stability of ¹⁷⁷Lu-B mixed 1:1 with stabilizing solutions a-c to provide
a) HSA with a final concentration of 50 mg/mL b) AA with a final concentration
of either 50 mg/mL or c) 25 mg/mL. Final ¹⁷⁷Lu-B concentration is 25 mCi/mL:

¹⁷⁷Lu-B
diluted 1:1 with the

| indicated Stabilizing | (% RCP) | | | | | | | | |
|---|---|---|---|---|---|---|---|---|---|
| Solution | 0-h | 3-h | 6-h | 9-h | 12-h | 24-h | 48-h | 72-h | 120-h |
| Stabilizing Solution a | 100 | 88.3 | 59.6 | 39.1 | 24.0 | 2.9 | 0 | 0 | 0 |
| 100 mg/mL HSA | | | | | | | | | |
| Stabilizing Solution b | 99.9 | 99.9 | 99.7 | 99.1 | 98.7 | 96.1 | 93.6 | 92.0 | 91.7 |
| 100 mg/mL AA | | | | | | | | | |
| Stabilizing Solution c | 99.9 | 99.9 | 99.8 | 99.1 | 98.1 | 96.0 | 92.5 | 92.8 | 92.2 |
| 50 mg/mL AA + BA | | | | | | | | | |

The results of Example 8 above indicate that either HSA alone or ascorbic acid alone could not maintain an RCP of >95% for times longer than 24 hours.

The results of Example 1-8 indicate that a Radiolysis Protecting Solution containing gentisic acid, ascorbic acid, Human Serum Albumin, benzyl alcohol and either cysteine, selenomethionine, or methionine and (ethanol in 0-05M citrate buffer) will stabilize ¹⁷⁷Lu-A or ¹⁷⁷Lu-B if added after labeling, and that such a mixture will provide better radiostability than any of the reagents when added in isolation.

Such an approach would require a two-vial kit, with one vial containing the reagents required to prepare the radiolabeled product; the other containing the Radiolysis Protecting Solution, which is added after complex formation. Several studies were therefore performed to try and find a single-vial kit, wherein both the reagents needed to form ¹⁷⁷Lu-A or ¹⁷⁷Lu-B and the reagents needed to stabilize the resulting complex against radiolysis were combined into a single vial.

### Example 9

### Preparation, Labeling Efficiency and Stability of ¹⁷⁷Lu-A When Prepared in the Presence of L-Cysteine Hydrochloride Monohydrate, Gentisic Acids Ascorbic Acid, L-Selenomethionine or D-Methionine (1 mg/mL) Individually, as Stabilizers

In this study, each of the reagents in the stabilizing buffer (cysteine, gentisic acid, ascorbic acid, selenomethionine and methionine was tested individually by adding 1.0 mg/mL of the individual reagent directly to radiolabeling reactions containing a small amount of radioactivity (3.5 mCi). None interfered with the labeling reaction, but only selenomethionine and methionine showed good protection over time at the low radioactivity levels used.

Each individual stabilizer was prepared at a concentration of 1 mg/mL in sodium acetate (NaOAc) buffer (0.2 M, pH 4.8). To lead-shielded 4-mL vials was added 200 μL of the individual NaOAc-stabilizer solutions, 2.72-3.64 mCi ¹⁷⁷LuCl₃ and 4.6-6 μg COMPOUND A (dissolved in water). The ratio of COMPOUND A to Lutetium was 3:1 for all samples. The reaction mixture was heated to 100° C. for 5 minutes, and then cooled for 5 minutes in an ambient-temperature water bath. To each sample, 10 μL of 2% Na₂EDTA.2H₂O in water was added, and then each was divided into two 100-μL aliquots. One aliquot was analyzed by HPLC (Method 1) and then stored at room temperature in a sealed lead container for 24 hours. The other aliquot was stored frozen (−10° C.) for 24 hours. Each sample was analyzed at t=24 h. The radiochemical purity (RCP) percentage data obtained are listed in Table 11.

TABLE 11
RCP Data for ¹⁷⁷Lu-A (2.7-3.7 mCi) when Prepared in the
Presence of L-Cysteine Hydrochloride Monohydrate, Gentisic
Acid, Ascorbic acid, L-Selenomethionine or D-Methionine
(1 mg/mL), Individually, as Stabilizers

| | | COMPOUND | RCP % | | |
|---|---|---|---|---|---|
| (1 mg/mL) | mCi | A Conc. | | t = 24 h | t = 24 h |
| Stabilizer | ¹⁷⁷Lu | (μg/mL) | t = 0 h | (room temp) | (frozen) |
| L-cysteine | 3.64 | 25.4 | 100 | 83.3 | 82.1 |
| Gentisic acid | 2.76 | 23.0 | 100 | 64.9 | 84.3 |
| Ascorbic acid | 3.19 | 30.0 | 98.7 | ND | ND |
| L-Se-Met | 2.72 | 23.0 | 100 | 100 | 99.2 |
| D-Methionine | 2.76 | 23.0 | 100 | 100 | 100 |

ND = not determined

The results demonstrate that none of the five stabilizers interferes with the labeling reaction and that each provides stability during the reaction at the 1-mg/mL concentration used. However, L-selenomethionine and D-methionine are better stabilizers than the others tested, at this concentration, during 24 hours of storage, both at room temperature and frozen. Data for the stored samples using ascorbic acid were not collected.

### Example 10

### Preparation, Labeling Efficiency and Stability of ¹⁷⁷Lu-A When Prepared in the Presence of L-Cysteine Hydrochloride Monohydrate, Gentisic Acid, Ascorbic Acid, L-Selenomethionine or D-Methionine (2.5 mg/mL), Individually, as Stabilizers

In Examples 10 and 11, reagents in the stabilizing buffer (cysteine, gentisic acid, ascorbic acid, selenomethionine or methionine were tested individually by adding 2.5 mg/mL (Example 10) or 5.0 mg/mL (Example 11) of the individual reagents directly to radiolabeling reactions containing a small amount of radioactivity (3.5 mCi). When the amount of stabilizers was increased to 2.5 mg/mL and 5 mg/mL to decrease the potential for radiolytic damage at high activity levels, it was found again that gentisic acid, ascorbic acid and cysteine could not provide adequate radioprotection for 24 hours, even at radioactivity amounts less than 5 mCi. Each stabilizer was prepared at a concentration of 2.5 mg/mL in sodium acetate (NaOAc) buffer (0.2 M, pH 4.8). To lead-shielded 4-mL vials was added 200 μL of the individual NaOAc-stabilizer solutions, 3.58 mCi ¹⁷⁷LuCl₃ (avg) and 5.08 μg COMPOUND A (dissolved in water). The ratio of COMPOUND A to Lutetium was 3:1 for all samples. The reaction mixtures were heated to 100° C. for 5 minutes, then cooled, treated with Na₂EDTA.2H₂O, subdivided and stored as described in Example 9. The radiochemical purity (RCP) percentage data obtained are listed in Table 12.

TABLE 12
RCP Data for ¹⁷⁷Lu-A when Prepared in the Presence
of L-Cysteine Hydrochloride Monohydrate, Gentisic
acid, Ascorbic acid, L-Selenomethionine or D-
Methionine (2.5 mg/mL) as Stabilizers

| | | COMPOUND | RCP % | | |
|---|---|---|---|---|---|
| (2.5 mg/mL) | mCi | A Conc. | | t = 24 h | t = 24 h |
| Stabilizer | ¹⁷⁷Lu | (μg/mL) | t = 0 h | (room temp) | (frozen) |
| L-Cysteine | 3.6 | 24.8 | 100 | 88.3 | 51.0 |
| Gentisic acid | 3.6 | 24.8 | 100 | 78.6 | 82.8 |
| Ascorbic acid | 3.6 | 24.8 | ND | 88.1 | 78.4 |
| L-Seleno- | 3.6 | 24.8 | 95.7 | 95.6 | 94.7 |
| Methionine | | | | | |
| D-Methionine | 3.6 | 24.8 | 100 | 99.6 | 100 |

ND = not determined

The results demonstrate that at the 2.5-mg/mL concentration, L-cysteine, gentisic acid and D-methionine do not interfere with the labeling reaction and provide stability during the reaction. L-Selenomethionine either interferes somewhat or provides less stability during the reaction. L-Selenomethionine and D-methionine are better stabilizers, at this concentration, during 24 hours of storage, both at room temperature and frozen. Data for the t=0 h sample using Ascorbic Acid was not collected.

### Example 11

### Preparation, Labeling Efficiency and Stability of ¹⁷⁷Lu-A When Prepared in the Presence of L-Cysteine Hydrochloride Monohydrate, Gentisic Acids Ascorbic Acids L-Selenomethionine or D-Methionine (5 mg/mL) as Stabilizers

Each stabilizer was prepared at a concentration of 5 mg/mL in sodium acetate (NaOAc) buffer (0.2 M, pH 4.8). To lead-shielded 4-mL vials were added 200 μL of the individual stabilizer solutions, 3.55 mCi ¹⁷⁷LuCl₃ (avg) and 5.44 μg COMPOUND A (dissolved in water). A second set of replicates of each sample was prepared, using the individual stabilizers. To these was added 4.37 mCi ¹⁷⁷LuCl₃ (avg) and 6.7 μg (avg) COMPOUND A (dissolved in water). The ratio of COMPOUND A to lutetium was 3:1 for all samples. The reaction mixture was heated to 100° C. for 5 minutes then cooled for 5 minutes in an ambient-temperature water bath. To each sample, 10 μL of 2% Na₂EDTA.2H₂O in water was added, then each was analyzed by HPLC (Method 1 for the first set of replicates; Method 2 for the second set of replicates). The second set of replicates were stored and analyzed again at t=24 h. The radiochemical purity (RCP) percentage data obtained are given in Table 13 below.

TABLE 13
RCP Data for ¹⁷⁷Lu-A when Prepared in the Presence
of L-Cysteine Hydrochloride Monohydrate, Gentisic acid,
Ascorbic acid, L-Selenomethionine or D-Methionine (5 mg/mL)
as Stabilizers

| | Replicate 1 | Replicate 2 | Replicate 2 |
|---|---|---|---|
| (5 mg/mL) | RCP % | RCP % | RCP% |
| Stabilizer | t = 0 h | t = 0 h | t = 24 h |
| L-cysteine | 93.3 | 91.0 | 67.4 |
| Gentisic acid | 93.9 | 96.6 | 75.2 |
| Ascorbic acid | 87.6 | 30.2 | 24.5 |
| L-Selenomethionine | 57.5 | 60.6 | 57.1 |
| D-Methionine | 100 | 97.6 | 80.7 |

The results demonstrate that at the 5-mg/mL concentration, D-methionine does not interfere with the labeling reaction and provides stability during the reaction. L-cysteine, gentisic acid, ascorbic acid and L-selenomethionine either interfere with the labeling reaction or provide less stability during the reaction. Reproducibility between replicates at the t=0 h time point was adequate for each stabilizer except ascorbic acid. Ascorbic acid and L-selenomethionine provided better stability during 24 hours of storage (as compared to their t=0 h RCP % values) than L-cysteine, gentisic acid or D-methionine.

### Example 12

### Stability of ¹⁷⁷Lu-A When Stabilized After Complex Preparation Using 2-Ethyl-4-Pyridinecarbothioaxide (Ethionamide), Trithiocyanuric Acid Trisodium Salt Nonahydrate, Sodium Dimethyldithiocarbamate Hydrate or Sodium Diethyldithiocarbamate Trihydrate as Stabilizers

Compounds containing the —C═S moiety [dithiocarbamates and ethionamide] were examined in this study. When added after complex preparation, the compounds ethionamide, tricyanuric acid, and dimethyldithiocarbamic acid and its diethyl analog all provided good radiostability.

Each individual stabilizer was prepared at a concentration of 10 mg/mL in water. Ethionamide was dissolved in EtOH. To a lead-shielded 4-mL vial was added 500 μL of NaOAc buffer (0.2M, pH 4.8), 19.6 mCi ¹⁷⁷LuCl₃ and 30 μg COMPOUND A (dissolved in water). The ratio of COMPOUND A to Lutetium was 3:1. The reaction mixture was heated to 100° C. for 5 minutes, then cooled for 5 minutes in an ambient-temperature water bath. After cooling, 20 μL of 2% Na₂EDTA.2H₂O in water was added, and then four 100-μL aliquots of the sample (2.78 mCi ¹⁷⁷Lu avg each) were transferred to individual autosampler vials. To an aliquot, 100 μL of one of the stabilizer solutions (1 mg of stabilizer) was added. Each aliquot was analyzed (t=0 h) by HPLC (Method 2) and stored at room temperature for 48 hours. All samples were analyzed again at t=24 h and 48 h. The radiochemical purity (RCP) percentage data obtained are listed in Table 14.

TABLE 14
RCP Data for ¹⁷⁷Lu-A When Stabilized After Complex
Preparation Using 2-Ethyl-4-pyridinecarbothioamide
(Ethionamide), Trithiocyanuric acid trisodium salt
nonahydrate, Sodium dimethyldithiocarbamate hydrate or Sodium
diethyldithiocarbamate trihydrate as Stabilizers. (13.9 mCi/mL)

| | RCP % | RCP % | RCP % |
|---|---|---|---|
| Stabilizer (5 mg/mL) | t = 0 h | t = 24 h | t = 48 h |
| Ethionamide | 97.4 | 97.0 | 94.7 |
| Trithiocyanuric acid trisodium salt | 96.9 | 95.9 | 100 |
| nonahydrate | | | |
| Sodium dimethyldithiocarbamate hydrate | 97.3 | 97.1 | 96.6 |
| Sodium diethyldithiocarbamate trihydrate | 97.8 | 97.1 | 96.2 |

The results demonstrate that, at a 5-mg/mL concentration, each of the stabilizers provided stability for ¹⁷⁷Lu-A at a radioconcentration of 13.9 mCi/mL for up to 48 hours of storage.

### Example 13

### Preparation, Labeling Efficiency and Stability of ¹⁷⁷Lu-A When Prepared in the Presence of 2-Ethyl-4-Pyridinecarbothioamide (Ethionamide), Trithiocyanuric Acid Trisodium Salt Nonahydrate, Sodium Dimethyldithiocarbamate Hydrate, or Sodium Diethyldithiocarbamate Trihydrate as Stabilizers

In Example 12, compounds containing the —C═S moiety [dithiocarbamates and ethionamide] were added after radiolabeling, and found to be effective radiostabilizers. In Example 13, these compounds were added directly to the reaction mixture before or at the time of radiolabeling.

10 mg/mL solutions of trithiocyanuric acid trisodium salt nonahydrate, sodium dimethyldithiocarbamate hydrate, and sodium diethyldithiocarbamate trihydrate were prepared by dissolving them in water. Ethionamide was prepared at a 10 mg/mL concentration by dissolving it in EtOH. To individual, lead-shielded, 4-mL vials were added 200 μL of NaOAc buffer (0.2M, pH 4.8), 100 μL of stabilizer solution (1 mg of stabilizer), 5.25 mCi ¹⁷⁷LuCl₃ (avg) and 8.7 μg (avg) COMPOUND A (dissolved in water). Another sample was prepared to which was added 100 μL of ethanol only (no stabilizer), for use as a control sample. The ratio of COMPOUND A to Lutetium was 3:1 for all samples. The reaction mixture was heated to 100° C. for 5 minutes then cooled for 5 minutes in an ambient-temperature water bath. To each sample, 10 μL of 2% Na₂EDTA.2H₂O in water was added, and then each was analyzed by HPLC (Method 2) and stored at room temperature for up to 96 hours. The radiochemical purity (RCP) percentage data obtained are listed in Table 15.

TABLE 15
RCP Data for ¹⁷⁷Lu-A When Prepared in the Presence of 2-Ethyl-4-
pyridinecarbothioamide (Ethionamide), Trithiocyanuric acid
trisodium salt nonahydrate, Sodium dimethyldithiocarbamate
hydrate, or Sodium diethyldithiocarbamate trihydrate as Stabilizers

| | RCP % | RCP % | RCP % |
|---|---|---|---|
| Stabilizer (˜3.33 mg/mL) | t = 0 h | t = 24 h | t = 96 h |
| Ethanol (no stabilizer) | 100 | 59.3 | — |
| Ethionamide | 100 | 98.1 | 94.6 |
| Trithiocyanuric acid trisodium salt | 100 | 100 | 0 |
| nonahydrate | | | |
| Sodium dimethyldithiocarbamate hydrate | 69.3 | 1.5 | — |
| Sodium diethyldithiocarbamate trihydrate | 52.8 | 0 | — |

The results demonstrate that, at a stabilizer concentration of 3.33-mg/mL, ethanol, ethionamide and trithiocyanuric acid trisodium salt nonahydrate did not interfere with the labeling reaction and each provided stability during the reaction. Sodium dimethyldithiocarbamate hydrate and sodium diethyldithiocarbamate trihydrate interfered with the reaction or provide less stability during the reaction. Ethionamide and trithiocyanuric acid trisodium salt nonahydrate provided stability for up to 24 hours and 96 hours of storage, respectively. In the case of trithiocyanuric acid trisodium salt nonahydrate, the drop in stability observed between 24 and 96 hours was probably due to an insufficient amount of the compound to maintain stability. In Example 12, a higher concentration of this compound did maintain stability for 48 hours.

### Example 14

### Preparation, Labeling Efficiency and Solution Stability of ¹⁷⁷Lu-A When Prepared in the Presence of Thiamine Hydrochloride, L-Glutathione, 3-Hydroxycinnayic Acid, 4-Hydroxyantipyrine, Acetylsalicylic Acids 2-Hydroxybenzothiazole or 2,1,3-Benzothiadiazole as Stabilizers

10 mg/mL solutions of thiamine hydrochloride and L-glutathione were prepared by dissolving them in water. 10 mg/mL solutions of 3-hydroxycinnamic acid, 4-hydroxyantipyrine and acetylsalicylic acid were prepared by dissolving them in 50% EtOH/water. 10 mg/mL solutions of 2-hydroxybenzothiazole and 2,1,3-benzothiadiazole were prepared by dissolving them in EtOH. To individual, lead-shielded 4-mL vials were added 200 μL of NaOAc buffer (0.2M, pH 4.8), 100 μL of stabilizer solution (1 mg of stabilizer), 5.28 mCi ¹⁷⁷LuCl₃ (avg) and 9.6 μg (avg) COMPOUND A (dissolved in water). The ratio of COMPOUND A to Lutetium was 3:1 for all samples. The reaction mixtures were heated, cooled, treated with Na₂EDTA.2H₂O and analyzed by HPLC as described in Example 13, then stored at room temperature for 72 hours, at which time all samples were analyzed again. The radiochemical purity (RCP) percentage data obtained are listed in Table 16.

TABLE 16
RCP Data for ¹⁷⁷Lu-A When Prepared and Stored in the Presence of
Thiamine Hydrochloride, L-Glutathione, 3-Hydroxycinnamic acid,
4-Hydroxyantipyrine Acetylsalicylic acid, 2-Hydroxybenzothiazole or
2,1,3-Benzothiadiazole as Stabilizers

| | | |
|---|---|---|
| | RCP (%) | RCP (%) |
| Stabilizer (3.33 mg/mL) | t = 0 h | t = 72 h |

| | | |
|---|---|---|
| Thiamine hydrochloride | 97.0 | 0 |
| L-Glutathione | 91.1 | 0 |
| 3-Hydroxycinnamic acid | 96.6 | 0 |
| 4-Hydroxyantipyrine | 99.9 | 0 |
| Acetylsalicylic acid | 73.0 | 0 |
| 2-Hydroxybenzothiazole | 96.2 | 9.6 |
| 2,1,3-Benzothiadiazole | 98.6 | 3.1 |

The results demonstrate that, at the 3.33-mg/mL concentration, thiamine hydrochloride, 3-hydroxycinnamic acid, 4-hydroxyantipyrine, 2-hydroxybenzothiazole and 2,1,3-benzothiadiazole do not significantly interfere with the ¹⁷⁷Lu-A labeling reaction and that they provide effective radiostability during the labeling reaction. L-Glutathione and acetylsalicylic acid either interfere with the labeling reaction or provide less stability during the reaction under the conditions tested. None of the stabilizers tested provided significant stability for up to 72 hours of storage.

### Example 15

In a following experiment, the dithiocarbamate 1-pyrrolidine dithiocarbamic acid, ammonium salt, which has not been previously evaluated as a radiostabilizer for radiodiagnostic or radiotherapeutic compounds, was added directly to the radiolabeling mixture. Surprisingly, unlike the dithiocarbamates tested in Examples 12 and 13, PDTC provided both excellent initial RCP and post-labeling stability. This result was very unexpected. Study of this compound was extended (in Example 18), where it was found that at 20 mg/mL, 100% RCP could be obtained for up to 48 hours.

### Preparation, Labeling Efficiency Determination and Solution Stability of ¹⁷⁷Lu-A Using 2-Ethyl-4-Pyridinecarbothioamide (Ethionaniide), 1-Pyrrolidine Dithiocarbamic Acid Ammonium Salt and 5-Thio-D-Glucose (5 mg/mL) as Stabilizers

5 mg/mL solutions of 1-pyrrolidine dithiocarbamic acid ammonium salt (PDTC) and 5-thio-D-glucose were prepared in sodium acetate buffer (0.2 M, pH 4.8). A 5 mg/mL solution of ethionamide was prepared in 25% EtOH/NaOAc buffer. To lead-shielded 4-mL vials were added 200 μL of the individual NaOAc-stabilizer solutions, 4.65-5.64 mCi ¹⁷⁷LuCl₃ and 7.1-8.5 μg COMPOUND A (dissolved in water). The ratio of COMPOUND A to Lutetium was 3:1 for all samples. The reaction mixtures were heated, cooled, treated with Na₂EDTA.2H₂O and analyzed by HPLC as described in Example 13, and then stored at room temperature for 24 hours, at which time all samples were analyzed again. The RCP data obtained are listed in Table 17.

TABLE 17
RCP Data for ¹⁷⁷Lu-A When Prepared in the Presence of 2-Ethyl-4-
pyridinecarbothioamide (Ethionamide), 1-pyrrolidine dithiocarbamic
acid ammonium salt (PDTC) or 5-Thio-D-glucose (5 mg/mL)
as Stabilizers

| | | COMPOUND | | |
|---|---|---|---|---|
| | mCi | A | RCP % | RCP % |
| Stabilizer (5 mg/mL) | ¹⁷⁷Lu | Conc. (μg/mL) | t = 0 h | t = 24 h |
| Ethionamide | 5.45 | 42.5 | 80.8 | 77.5 |
| 1-pyrrolidine dithiocarbamic | 5.64 | 42.5 | 100 | 99.9 |
| acid ammonium salt (PDTC) | | | | |
| 5-Thio-D-glucose | 4.65 | 35.5 | 81.3 | 38.0 |

The results demonstrate that PDTC does not interfere with the ¹⁷⁷Lu-A labeling reaction and provides stability during the reaction at the 5-mg/mL concentration. In contrast, ethionamide (in 25% EtOH/NaOAc) and 5-thio-D-glucose either interfere with the labeling reaction or provide less stability during the reaction under the conditions tested. Ethionamide and PDTC are better stabilizers than 5-thio-D-glucose (as compared to their t=0 h RCP % values) during 24 hours of storage.

### Example 16

### Stability of ¹⁷⁷Lu-A When Stabilized After Complex Preparation Using Cystamine Dihydrochloride, L-Cysteine Ethyl Ester Hydrochloride, L-Cysteine Diethyl Ester Dihydrochloride, L-Cysteine Methyl Ester Hydrochloride, L-Cysteine Dimethyl Ester Dihydrochloride or L-Cysteinesulfinic Acid Monohydrate (5 mg/mL) as Stabilizers

In this study, sulfur-containing compounds were tested. Cysteine has been used as an antioxidant for many drugs that contain oxidizable residues. However, cysteine alone was found to interfere with radiolabeling if added directly to reaction mixtures for the preparation of ¹⁷⁷Lu-A (Example 11), and to be partially effective if added after the ¹⁷⁷Lu complex was formed. Surprisingly, the cysteine methyl and ethyl esters, which have not previously been reported as stabilizers in radiopharmaceuticals, provided better radiostabilization under the conditions tested.

Solutions of each individual stabilizer (10 mg/mL) were prepared in water. To a lead-shielded 4-mL vial was added 300 μL of NaOAc buffer (0.2M, pH 4.8), 29.6 mCi ¹⁷⁷LuCl₃ and 41.4 μg COMPOUND A (dissolved in water). The ratio of COMPOUND A to Lutetium was 3:1. The reaction mixture was heated, cooled, treated with Na₂EDTA.2H₂O and analyzed by HPLC as described in Example 13. Seven 50-μL aliquots (3.34 mCi ¹⁷⁷Lu avg each) were transferred to individual HPLC vials. To one aliquot, 50 μL of water was added for use as a control sample (no stabilizer). To the other six aliquots, 50 μL of an individual stabilizer solution (0.5 mg of stabilizer) was added, and then each was analyzed by HPLC (Method 2). The control sample, and L-cysteine ethyl ester hydrochloride and L-cysteine methyl ester hydrochloride samples were analyzed again after 24 hours of storage at room temperature. The RCP data obtained are listed in Table 18.

TABLE 18
RCP Data for ¹⁷⁷Lu-A When Stabilized After Complex
Preparation Using Cystamine dihydrochloride, L-Cysteine ethyl
ester hydrochloride, L-Cysteine diethyl ester dihydrochloride,
L-Cysteine methyl ester hydrochloride, L-Cysteine dimethyl
ester dihydrochloride, or L-Cysteinesulfinic acid monohydrate
(5 mg/mL) as Stabilizers

| | Time of | | |
|---|---|---|---|
| | analysis | RCP | RCP |
| | after | (%) | (%) |
| Stabilizer (5 mg/mL) | preparation | t = 0 h | t = 24 h |
| None (control sample) | 0 hr | 93.2 | 0 |
| Cystamine dihydrochloride | 1 hr | 66.2 | — |
| L-cysteine ethyl ester hydrochloride | 1.5 hrs | 91.5 | 73.2 |
| L-cysteine methyl ester hydrochloride | 2.5 hrs | 90.4 | 74.4 |
| L-cysteine diethyl ester dihydrochloride | 2 hrs | 61.5 | — |
| L-cysteine dimethyl ester | 3 hrs | 70.1 | — |
| dihydrochloride | | | |
| L-cysteinesulfinic acid monohydrate | 3.5 hrs | 75.0 | — |

The results demonstrate that at the 5-mg/mL concentration, L-cysteine ethyl ester hydrochloride and L-cysteine methyl ester hydrochloride provide better radiostability for ¹⁷⁷Lu-A than do the other stabilizer solutions tested.

### Example 17

### Preparation, Labeling Efficiency Determination and Solution Stability of ¹⁷⁷Lu-A Using L-Cysteine Ethyl Ester Hydrochloride and L-Cysteine Methyl Ester Hydrochloride (5 mg/mL) as Stabilizers

Solutions of L-cysteine ethyl ester hydrochloride and L-cysteine methyl ester hydrochloride (5 mg/mL) were prepared by dissolving them in NaOAc buffer (0.2 M, pH 4.8). To lead-shielded 4-mL vials were added 200 μL of the individual NaOAc-stabilizer solutions, 4.80 mCi ¹⁷⁷LuCl₃ and 7.26 μg COMPOUND A (dissolved in water). The ratio of COMPOUND A to Lutetium was 3:1 for all samples. The reaction mixtures were heated, cooled, treated with Na₂EDTA.2H₂O and analyzed by HPLC as described in Example 13, and then each was stored at room temperature for 72 hours. Each sample was analyzed by HPLC (Method 2) at t=0, 24, 48 and 72 h. The RCP data obtained are listed in Table 19.

TABLE 19
RCP Data for ¹⁷⁷Lu-A When Prepared in the Presence of
L-Cysteine ethyl ester hydrochloride or L-Cysteine methyl
ester hydrochloride (5 mg/mL) as Stabilizers

| | RCP % | RCP % | RCP % | RCP % |
|---|---|---|---|---|
| Stabilizer (5 mg/mL) | t = 0 h | t = 24 h | t = 48 h | t = 72 h |
| L-cysteine ethyl ester | 100 | 96.5 | 93.5 | 87.4 |
| hydrochloride | | | | |
| L-cysteine methyl ester | 100 | 97.1 | 93.7 | 87.2 |
| hydrochloride | | | | |

The results demonstrate that, at the 5-mg/mL concentration, both stabilizers provide adequate ¹⁷⁷Lu-A stability for up to 24 hours.

### Example 18

### Preparation, Labeling Efficiency and Solution Stability of ¹⁷⁷Lu-A Prepared in the Presence of 1-Pyrrolidine Dithiocarbamic Acid Ammonium Salt (0-20 mg/mL)

Solutions of 1-pyrrolidine dithiocarbamic acid ammonium salt (PDTC) were prepared at concentrations of 20-, 10-, 5- and 1 mg/mL by dissolving it in a sodium acetate (NaOAc) buffer solution (0.2 M, pH 4.8). To lead-shielded 300-μL sample vials were added, individually, 50-μL aliquots of the PDTC-NaOAc buffer solutions, including an aliquot of the NaOAc buffer only, to serve as a control sample. To each buffer aliquot was added 9.95 mCi ¹⁷⁷LuCl₃ (avg) and 17.2 μg COMPOUND A (dissolved in water). The molar ratio of COMPOUND A:Lu (total Lu) for each sample was 3:1. During the reaction, in each sample, the concentration of COMPOUND A was 287-μg/mL and the activity concentration was 167-mCi/mL. The samples were heated to 100° C. for 5 minutes, then cooled for 5 minutes in an ambient-temperature water bath. To each sample, 10 μL of 2% EDTA in water was added, and then each was analyzed by HPLC (Method 3) over 48 hours. At t=0, the radioactivity concentration was 143 mCi/mL. The table below shows the results obtained.

TABLE 20
RCP Data for ¹⁷⁷Lu-A When Prepared in the Presence of 1-pyrrolidine
dithiocarbamic acid ammonium salt (PDTC) at 0-20 mg/mL

| | RCP (%) | | | | | |
|---|---|---|---|---|---|---|
| PDTC Concentration | 0-h | 3-h | 6-h | 12-h | 24-h | 48-h |
| (None - NaOAc only) Control | 100 | 30.7 | 0 | 0 | — | — |
| 20 mg/mL (1 mg) | 100 | 100 | 100 | 100 | 100 | 100 |
| 10 mg/mL (0.5 mg) | 100 | 100 | 100 | 100 | 100 | 100 |
| 5 mg/mL (0.25 mg) | 100 | 100 | 100 | 100 | 0 | — |
| 1 mg/mL (0.05 mg) | 100 | 100 | 17.2 | 0 | 0 | — |

These results were obtained in the absence of any other stabilizer, and indicate that PDTC can provide exceptional radiostabilization. As the stabilizer was present during the labeling reaction, it indicates that a single-vial formulation using this reagent should be feasible. Additionally, this experiment demonstrates that an increased amount of stabilizer extends the duration of stability.

### Example 19

### Preparation, Labeling Efficiency and Solution Stability of ¹⁷⁷Lu-B Prepared in the Presence of 1-Pyrrolidine Carbodithioic Acid Ammonium Salt (PDTC), Selenomethionine (Se-Met), Cysteine (Cys) or Cysteine Ethyl Ester (CEE)

PDTC: In this study, ¹⁷⁷Lu-B was formulated as follows: To a 5-mL glass vial was added 5 mg of PDTC dissolved in 1 mL 0.2 M NaOAc buffer (pH 4.8), 15 μL (44 mCi) of ¹⁷⁷LuCl₃ and 30 μL of a 5 mg/mL solution of COMPOUND B in 0.01N HCl. The reaction vial was crimp-sealed and heated at 100° C. for 5 min. After cooling with a water bath, 1 mL of Bacteriostatic 0.9% NaCl, Injection containing 0.9% Benzyl Alcohol and 1 mg/mL Na₂EDTA.2H₂O was added. The sample was stored in an autosampler in which the temperature is 6° C. higher than room temperature, and analyzed by RP-HPLC for up to 24 hours. The table below shows the results obtained.

L-Selenomethionine: ¹⁷⁷Lu-B was prepared, diluted and analyzed as described above, but 5 mg of L-Se-Met was used in place of PDTC, the heating time was 10 minutes, and the quantity of radioactivity used was 52 mCi.

L-cysteine ethyl ester, HCl: ¹⁷⁷Lu-B was prepared, diluted and analyzed as described above, but 5 mg of L-CEE, hydrochloride salt was used in place of PDTC, the heating time was 8 minutes and the quantity of radioactivity used was 50 mCi.

L-cysteine.HCl.H₂O: ¹⁷⁷Lu-B was prepared, diluted and analyzed as described above, but 5 mg of L-Cys HCl.H₂O was used in place of PDTC, the heating time was 8 minutes and the quantity of radioactivity used was 53 mCi.

TABLE 21
RCP Data for ¹⁷⁷Lu-B When Prepared in the Presence of PDTC, L-Selenomethionine,
L-Cysteine ethyl ester or L-Cysteine•HCl•H₂O

¹⁷⁷Lu-B

| formulated to contain | (% RCP) | | | | | | |
|---|---|---|---|---|---|---|---|
| 5 mg of the following | | | | | | | RCP Drop over |
| stabilizing compound | 0-h | 3-h | 6-h | 9-h | 12-h | 24-h | 24 hr |
| PDTC | 95.0 | | 94.7 | | | 94.0 | 1 |
| L-Selenomethionine | 95.0 | 94.6 | 94.4 | 93.3 | 92.6 | 90.6 | 4.4 |
| L-Cysteine ethyl | 98.4 | 96.9 | 94.7 | 92.8 | 92.2 | 85.1 | 13.3 |
| ester•HCl | | | | | | | |
| L-Cysteine•HCl•H₂O | 99.1 | 94.7 | 87.3 | 79.4 | 73.6 | 50.9 | 48.2 |

These data indicate that under the conditions tested, all compounds provided some radiostabilization, when compared to historical controls with no stabilizer added, and that PDTC and L-Selenomethionine were the most efficacious of the compounds tested. The fact that PDTC could be added directly to reaction mixtures for the preparation of Lu and Indium complexes [data not shown] without inhibiting complex formation is unexpected. Compounds such as diethyl dithiocarbamate, dimethyl dithiocarbamate and others, when added to Tc formulations, have been found to form complexes (e.g., Tc NOEt) wherein the radiometal coordinates to the dithiocarbamate ligand. Likewise, several reports of Indium complexes of dithiocarbamate ligands have been published.

### Example 20

### Determination of the Effects of a Contaminant Metal (Zinc) During the Reaction of ¹⁷⁷Lu-B With and Without 1-Pyrrolidine Dithiocarbamic Acid Ammonium Salt in the Reaction Buffer

During the investigations with PDTC, it was found that its addition to reaction mixtures containing ¹⁷⁷LuCl₃ provided a very unexpected benefit. At times, ¹⁷⁷LuCl₃ isotope solutions are contaminated with non-radioactive metals that can interfere with radiolabeling. These metals (which may include, for example Zn, Cu, Ca and Fe among others), can compete with ¹⁷⁷Lu for the chelator, thus lowering reaction yields by consuming ligand so it is unavailable for chelation to ¹⁷⁷Lu. Studies of the labeling yield of ¹⁷⁷Lu A in the presence of PDTC with and without added Zinc show clearly that addition of PDTC to reaction mixtures containing added Zn prevents interference of this contaminating metal.

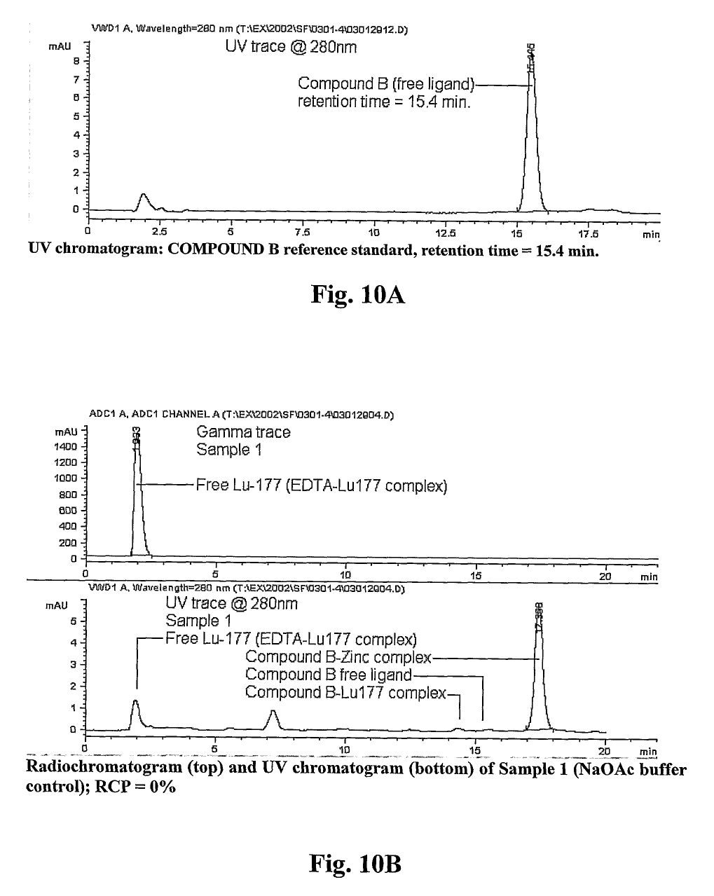

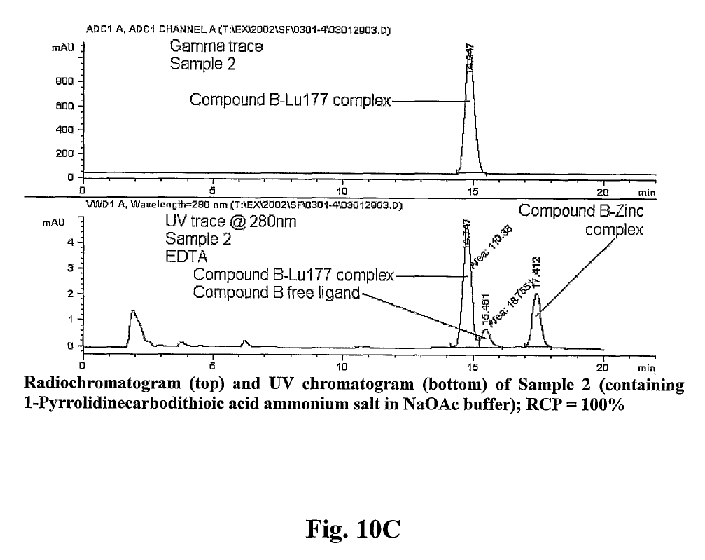

A 10-mg/mL solution of 1-pyrrolidine dithiocarbamic acid ammonium salt was prepared by dissolving it in sodium acetate buffer (0.2 M, pH 4.8). To a lead-shielded, 300-μL sample vial was added 86.5 μL of the NaOAc buffer solution, 13.7 mCi ¹⁷⁷LuCl₃, 0.6525 μg zinc (6.52 μL of a 100-μg/mL zinc plasma standard solution) and 15 μg COMPOUND B (dissolved in water). This was labeled as ‘Sample 1.’ To another lead-shielded, 300-μL sample vial was added 86.5 μL of the 10-μg/mL 1-pyrrolidine dithiocarbamic acid ammonium salt/NaOAc buffer solution, 13.8 mCi ¹⁷⁷LuCl₃, 0.6525 μg zinc and 15 μg COMPOUND B. This was labeled as ‘Sample 2.’ The concentration of COMPOUND B in each sample was 150 μg/mL and the molar ratio of COMPOUND B:¹⁷⁷Lu:Zinc for each sample was 3:1:3. The samples were heated to 100° C. for 5 min, and then cooled for 5 min in an ambient-temperature water bath. To each sample, 10 μL of 2% Na₂EDTA.2H₂O in water was added, and then each was analyzed by HPLC, using HPLC Method 5. FIG. 10 shows the results obtained.

FIG. 10A shows an HPLC chromatogram of COMPOUND B (UV), which has a retention time of 15.4 min. in the system used.

FIG. 10B shows a radiochromatogram (top) and UV chromatogram (bottom) of Sample 1 (control reaction; no PDTC); which was obtained following the reaction of COMPOUND B with ¹⁷⁷Lu in the presence of zinc. The resulting RCP was 0%. The primary product formed was the zinc complex of COMPOUND B, which has a retention time of 17.3 minutes. Very little COMPOUND B remains, and very little ¹⁷⁷Lu-B was formed.

FIG. 10C shows a radiochromatogram (top) and UV chromatogram (bottom) of Sample 2, which was obtained following the reaction of COMPOUND B with ¹⁷⁷Lu in the presence of zinc and PDTC. The resulting RCP=100%.

The results illustrated in FIGS. 10A-10C demonstrate that 1-pyrrolidine dithiocarbamic acid ammonium salt is effective in serving to scavenge adventitious trace metals in the reaction mixture, as radiochemical purity obtained is dramatically improved when PDTC is added to labeling reactions containing an excess of zinc.

### Example 21

### Preparation, Labeling Efficiency Determination and Solution Stability of ¹¹¹In-B Using Selenomethionine (2.5 mg/mL) as Stabilizer

A solution of L-selenomethionine (20 mg/mL) was prepared by dissolving it in NaOAc buffer (0.2 M, pH 4.0). To a lead-shielded 2-mL vial was added 111 μL of NaOAc buffer (0.2 M, pH 4.0), 25 μL selenomethionine solution (0.5 mg of Se-Met), 4 μL of COMPOUND B (20 μg in 0.01 N HCl) and 1.0 mCi ¹¹¹InCl₃ in 60 μL of 0.05 N HCl. A control reaction was run containing all reagents above, but substituting NaOAc buffer for the Se-Met solution. The reaction mixtures were heated at 100° C. for 15 minutes, and then cooled for 1 minute in an ambient-temperature water bath. To each sample, 200 μL of stabilizing solution (0.2% HSA, 5% ascorbic acid, 0.9% benzyl alcohol, 20 mM Se-Met in 50 mM citrate buffer, pH 5.3) and 2 μL of 1% Na₂EDTA.2H₂O in water were added, and then each was analyzed stored at room temperature for up to 6 hours and analyzed by HPLC as described below. The RCP data obtained are listed in Table 21. HPLC conditions: Vydac C18 column, 4.6×250 mm, 5 μM, 1.5 mL/min flow rate at 30° C. Solvent A: 0.1% TFA in water; Solvent B: 0.085% TFA in acetonitrile. Gradient: 80% A/20% B isocratic for 20 min, ramp up to 40% A/60% B in 5 min, hold for 5 min, return to 80% A/20% B in 5 min.

TABLE 22
RCP Data for ¹¹¹In-B When Prepared in the
Presence of Selenomethionine (2.5 mg/mL)

RCP, %

| Stabilizer added | t = 0 | t = 6 h |
|---|---|---|
| None (NaOAc buffer only) | 94.7 | 93.2 |
| 2.5 mg/mL Se-Met | 98.3 | 96.6 |

These results demonstrate that selenomethionine can be used for radiostabilization of ¹¹¹In B, as the radiochemical purity in the-reaction mixture where selenomethionine was added was higher than that obtained in the control reaction without stabilizer.

### Example 22

### Preparation, Labeling Efficiency Determination and Solution Stability of ¹⁷⁷Lu-B Using Selenomethionine and Sodium Ascorbate as Stabilizers

In this study, ¹⁷⁷Lu-B was formulated as follows: To a 5-mL glass vial was added 2.94 mg of L-Selenomethionine dissolved in 1 mL of 0.2 M NaOAc buffer (pH 4.8), 25 μL (110.5 mCi) of ¹⁷⁷LuCl₃ and 30 μL of a 5 mg/mL solution of COMPOUND B in 0.01N HCl. The reaction vial was crimp-sealed and heated at 100° C. for 10 min. After the reaction vial was cooled to room temperature in a water bath, 4 mL of Bacteriostatic 0.9% NaCl, Injection containing 0.9% Benzyl Alcohol, 50 mg/mL Sodium Ascorbate and 1 mg/mL Na₂EDTA.2H₂O was added. The sample was stored in an autosampler in which the temperature is ˜6° C. higher than room temperature, and analyzed by RP-HPLC for up to 120 hours Table 23 below shows the results obtained.

TABLE 23
RCP Data for ¹⁷⁷Lu-B When Prepared in the Presence
of L-Selenomethionine (2.94 mg/mL).

| Time post-reconstitution (Hours) | RCP (%) |
|---|---|
| 0 | 99.5 |
| 2 | 99.6 |
| 3 | 99.5 |
| 6 | 99.0 |
| 9 | 99.4 |
| 12 | 99.2 |
| 24 | 99.4 |
| 120 (5 days) | 99.8 |

These results indicate that both excellent labeling efficiency and excellent post-reconstitution stability can be obtained using the conditions described above, namely adding 2.94 mg Se-Met to the reaction mixture during complex formation, followed by 4 mL of a saline solution containing sodium ascorbate and benzyl alcohol immediately after complex formation. There was no observed degradation over 5 days of storage at room temperature. Similar results were obtained when the quantity of selenomethionine was reduced to 1.0 mg.

### Example 23

### Determination of the Effect of Benzyl Alcohol on the Recovery of ¹⁷⁷Lu-B

Two radiolysis protecting solutions were prepared as follows
Stabilizer Solution A: One part 500 mg/ml L-Ascorbic acid, pH 5.7 containing 0.25 mg/ml Na₂-EDTA was diluted with 9 parts of normal saline solution [no benzyl alcohol].
Stabilizer Solution B: One part 500 mg/ml L-Ascorbic acid, pH 5.7 containing 0.25 mg/ml Na₂-EDTA was diluted with 9 parts of Bacteriostatic saline, which contained 0.9% (w/v) benzyl alcohol.

100 μL aliquot of 0.2M NaOAc buffer, pH 4.8 containing 1 mg/mL L-selenomethionine and 13 μg of Compound B was added to each of two 2-mL sample vials, designated Sample 1 and Sample 2, respectively. Approximately 10 mCi of ¹⁷⁷LuCl₃ was added to each vial and the samples were heated at 100° C. for 10 minutes in a temperature-controlled heating block. They were then removed and cooled in an ambient temperature water bath for 5 minutes. After cooling, 400 μL of Solution A was added to Sample 1, and 400 μL of Solution B was added to Sample 2.

Both samples were analyzed by HPLC using Method 3 and allowed to stand at room temperature for 24 hours. At the end of this time, RCP analysis was repeated, and then the entire solution was removed from each vial. The amount of radioactivity remaining in each vial and the amount of radioactivity removed were determined using a dose calibrator. The percentage of radioactivity recovered from each vial was calculated as the mCi recovered from the vial/total activity [solution and vial] X100. The results observed are shown in Table 24.

TABLE 24
Comparision of RCP and % Recovery of ¹⁷⁷Lu-B
in the presence and absence of benzyl alcohol

| | RCP (%) | RCP (%) | Recovery | % remaining |
|---|---|---|---|---|
| | t = 0 | t = 24 hr | (%) | in vial* |
| Sample-01 | ˜100% | ˜100% | 85.3% | 14.7% |
| [no benzyl alcohol] | | | | |
| Sample-02 | ˜100% | ˜100% | 96.7% | 3.3% |
| [with benzyl alcohol] | | | | |

*% of the radioactivity remaining in the glass vial, unremovable

These results demonstrate that the addition of benzyl alcohol to the stabilizer solution improved recovery of radioactivity from the vial significantly. This is important, as if a significant amount of the radioactivity cannot be removed from the vial, then extra radioactivity must be added to offset this loss. It is highly advantageous to have recovery be as high as possible.

### Example 24

### Evaluation of the Use of +2 Sulfur Complexes to Convert Methionine Oxide Residues to Methionyl Residues in Radiolabeled Peptides

Sulfur compounds, particularly thiols, in the oxidation state +2 were evaluated for the ability to convert methionine oxide residues to the reduced methionyl form. To perform this study, the methionine oxidized form of Compound B was synthesized. This oxidized compound is referred to as Compound C. Compound C was radiolabelled to form ¹⁷⁷Lu-C, which was mixed with various +2 sulfur derivatives, stored at room temperature and analyzed over time to determine how much methionine oxide in the peptide had been converted to methionine.

The solutions tested were:
1. 2% Mercaptoethanol [ME] in 0.1 M, pH 5.0 citrate buffer.
2. 20 mg/ml L-Cysteine.HCl.H₂O [Cys], in 0.1 M, pH 5.0 citrate buffer; final pH of ˜3.5.
3. 20 mg/ml DL-Dithiothreitol [DTT] in 0.1 M, pH 5.0 Citrate buffer; final pH of ˜5.0.
4. 20 mg/ml L-Methionine [Met] in 0.1 M, pH 5.0 citrate buffer.
5. 20 mg/ml L-Selenomethionine [Se-Met] in 0.1 M, pH 5.0 citrate buffer.

Mercaptoethanol, cysteine and dithiothreitol are thiols, methionine is a thioether, and selenomethionine is an organic 2+selenium compound. The latter two solutions were used as controls.

Preparation of ¹⁷⁷Lu-C: In a 2-mL glass vial, 200 μl of 0.2 M, pH 4.8 NaOAc buffer, 30 μg Compound C [in 30 uL of 0.01 N HCl] and 5.6 mCi ¹⁷⁷LuCl₃ were added. After incubation at 85° C. for 10 min, the reaction vial was cooled to room temperature with a water bath, and then 20 μl of 2% EDTA was added to challenge any free Lu-177 that remained.

Sample preparation: Aliquots [40 μl, 0.75 mCi] of this reaction solution were mixed with a 100 μl aliquot of one of the solutions above, e.g., 20 mg/ml Cys; DTT; Met; Se-Met; or 2% ME. The solutions were stored at room temperature over time and analyzed by RP-HPLC at 1 and 3 days. The results obtained are shown in Table 25 below.

TABLE 25
Percentage (%) of ¹⁷⁷Lu-C converted [reduced] to¹⁷⁷Lu-B,
following room temperature storage in the presence of
Cys, DTT, ME, Met, or Se-Met for 1 to 3 days

| | % Conversion in | % Conversion in |
|---|---|---|
| | 1 day | 3 days |
| L-Cysteine (Cys) | 5.6 | 12.8 |
| DL-Dithiothreitol (DTT) | 14.1 | 18.9 |
| Mercaptoethanol (ME) | 9.7 | 17.1 |
| L-Methionine (Met) | 0 | 0 |
| L-Selenomethionine (Se-Met) | 0 | 0 |

This result is significant, as it indicates that Cys, DTT and ME, all thiol-containing compounds, are capable of reducing an oxidized methionyl residue in a radiolabeled peptide back to its reduced [methionyl] form. In formulations for the preparation of ¹⁷⁷Lu-A or ¹⁷⁷Lu-B, it is clear that addition of Cys, DTT or ME (or other thiol) can serve to protect these compounds from oxidation by reversing any methionine oxidation that occurs.

1-156. (canceled)

157. A stabilized radiopharmaceutical composition comprising:
(a) a diagnostic or therapeutic radionuclide, optionally complexed to a chelator; and
(b) a stabilizer selected form the group consisting of: a stabilizer comprising a water-soluble organic compound containing selenium in the +2 oxidation state; a stabilizer comprising a composition comprising ascorbic acid or a pharmaceutically salt thereof, gentisic acid or a pharmaceutically salt thereof, human serum albumin, and benzyl alcohol; a stabilizer comprising a dithiocarbamate compound; and a stabilizer comprising a water soluble compound containing sulfur in the +2 oxidation state.

158. A stabilized radiopharmaceutical composition comprising:
(a) a metal chelator complexed with a radionuclide;
(b) an optional linking group and a targeting molecule; and
(c) a stabilizer selected from the group consisting of: a stabilizer comprising a water-soluble organic compound containing selenium in the +2 oxidation state; a stabilizer composition comprising ascorbic acid or a pharmaceutically salt thereof, gentisic acid or a pharmaceutically salt thereof, human serum albumin, and benzyl alcohol; a stabilizer comprising dithiocarbamate compound; and a stabilizer comprising a water soluble compound containing sulfur in the +2 oxidation state.

159. A stabilized radiopharmaceutical composition comprising:
(a) a compound of the general formula:
M—N-Q
wherein
M is a metal chelator complexed with a radionuclide;
N is an optional linker;
Q is a targeting molecule; and
(b) a stabilizer selected from the group consisting of: a stabilizer comprising a water-soluble organic compound containing selenium in the +2 oxidation state; a stabilizer composition comprising ascorbic acid or a pharmaceutically salt thereof, gentisic acid or a pharmaceutically salt thereof, human serum albumin, and benzyl alcohol; a stabilizer comprising a dithiocarbamate compound; and a stabilizer comprising a water soluble compound containing sulfur in the +2 oxidation state.

160. A stabilized radiopharmaceutical composition comprising:
(a) a compound of the general formula:
M—N—O—P-Q
wherein
M is a metal chelator complexed with a radionuclide;
N is 0, an alpha amino acid, a non-alpha amino acid, or other linking group;
O is an alpha amino acid, or a non-alpha amino acid;
P is 0, an alpha amino acid, a non-alpha amino acid, or other linking group;
Q is a targeting molecule;
wherein at least one of N, O or P is a non-alpha amino acid; and
(b) a stabilizer selected from the group consisting of: a stabilizer comprising a water-soluble organic compound containing selenium in the +2 oxidation state; a stabilizer composition comprising ascorbic acid or a pharmaceutically salt thereof, gentisic acid or a pharmaceutically salt thereof, human serum albumin, and benzyl alcohol; a stabilizer comprising dithiocarbamate compound; and a stabilizer comprising a water soluble compound containing sulfur in the +2 oxidation state.

161. A stabilized radiopharmaceutical composition comprising:
(a) a compound of the general formula:
M—N—O—P-Q
wherein
M is a metal chelator complexed with a radionuclide;
N is 0, an alpha amino acid, a substituted bile acid, or other linking group;
O is an alpha amino acid, or a substituted bile acid;
P is 0, an alpha amino acid, a substituted bile acid, or other linking group;
Q is a targeting molecule;
wherein at least one of N, O or P is a substituted bile acid; and
(b) a stabilizer selected from the group consisting of: a stabilizer comprising a water-soluble organic compound containing selenium in the +2 oxidation state; a stabilizer composition comprising ascorbic acid or a pharmaceutically salt thereof, gentisic acid or a pharmaceutically salt thereof, human serum albumin, and benzyl alcohol; a stabilizer comprising a dithiocarbamate compound; and a stabilizer comprising a water soluble compound containing sulfur in the +2 oxidation state.

162. A stabilized radiopharmaceutical composition according to any one of claims 157 to 161 wherein the stabilizer composition comprising ascorbic acid or a pharmaceutically salt thereof, gentisic acid or a pharmaceutically salt thereof, human serum albumin, and benzyl alcohol, further comprises selenomethionine, selenocysteine, methionine, cysteine or derivatives thereof.

163. A stabilized radiopharmaceutical composition according to any one of claims 157 to 161, wherein the water-soluble compound containing selenium in the +2 oxidation state and is selected from the group consisting of selenomethionine, selenocysteine or derivatives thereof.

164. A stabilized radiopharmaceutical composition according to any one of claims 157 to 161, wherein the stabilizer comprising a dithiocarbamate compound comprises a compound of the formula: 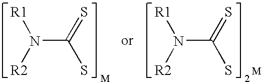
wherein R1 and R2 are each independently H; C₁-C₈ alkyl; —OR3, wherein R3 is C₁-C₈ alkyl; or benzyl, either unsubstituted or optionally substituted with water solubilizing groups; or wherein R1, R2, and N combined together form 1-pyrrolidinyl-, piperidino-, morpholino-, 1-piperazinyl-; and
M is H⁺, Na⁺, K⁺, NH₄⁺, N-methylglucamine, or other pharmaceutically acceptable +1 ion, in the +1 oxidation state; or
M is Mg²⁺ or Ca²⁺, or other physiologically acceptable +2 metal ion, in the +2 oxidation state.

165. A stabilized radiopharmaceutical composition according to any one of claims 157 to 161, wherein the stabilizer comprising a dithiocarbamate compound is selected from the group consisting of 1-pyrrolidine dithiocarbamic acid ammonium salt, sodium diethyldithiocarbamate trihydrate, sodium dimethyldithiocarbamate hydrate, and combinations thereof.

166. A stabilized radiopharmaceutical composition according to any one of claim 157 to 161, wherein the stabilizer comprising a water soluble compound containing sulfur in the +2 oxidation state comprises cysteine or a derivative thereof, mercaptoethanol, dithiolthreitol, or pharmaceutically acceptable salts thereof.

167. A stabilized radiopharmaceutical composition according to any one of claims 157 to 161, wherein the stabilizer comprising a water soluble compound containing sulfur in the +2 oxidation state comprises a cysteine derivative selected from the group consisting of cystamine dihydrochloride, cysteine hydrochloride monohydrate, cysteine ethyl ester hydrochloride, cysteine diethyl ester dihydrochloride, cysteine methyl ester hydrochloride, cysteine dimethyl ester dihydrochloride, cysteinesulfinic acid monohydrate, 5-thio-d-glucose, reduced 1-glutathione, and combinations thereof.

168. A stabilized radiopharmaceutical composition according to any one of claims 158 to 161, wherein the linker or linking group is a peptide, a hydrocarbon linking group or a combination thereof.

169. A stabilized radiopharmaceutical composition according to any one of claims 157 to 161, wherein the metal chelator is selected from the group consisting of DTPA, DOTA, DO3A, HP-DO3A, PA-DOTA, MeO-DOTA, MX-DTPA, EDTA, TETA, EHPG, HBED, NOTA, DOTMA, TETMA, PDTA, TTHA, LICAM, MECAM, CMDOTA, PnAO, oxa-PnAO, N,N-dimethylGly-Ser-Cys; N,N-dimethylGly-Thr-Cys; N,N-diethylGly-Ser-Cys; N,N-dibenzylGly-Ser-Cys, N,N-dimethylGly-Ser-Cys-Gly; N,N-dimethylGly-Thr-Cys-Gly; N,N-diethylGly-Ser-Cys-Gly; and N,N-dibenzylGly-Ser-Cys-Gly.

170. A stabilized radiopharmaceutical composition according to any one of claims 158 to 161, wherein the targeting molecule is a targeting peptide.

171. A stabilized radiopharmaceutical composition according to any one of claims 158 to 161, wherein the targeting molecule is selected from the group consisting of LHRH, insulin, oxytocin, somatostatin, NK-1, VIP, Substance P, NPY, endothelin A, endothelin B, bradykinin, interleukin-1, EGF, CCK, galanin, MSH, Lanreotide, Octreotide, Maltose, arginine-vasopressin, a GRP receptor targeting molecule, and analogs and derivatives thereof.

172. A stabilized radiopharmaceutical composition according to any one of claims 158 to 161, wherein the targeting molecule is a GRP receptor targeting molecule, which is optionally an agonist or a peptide which confers agonist activity.

173. A stabilized radiopharmaceutical composition according to any one of claims 158 to 161, wherein the targeting molecule is a GRP receptor targeting molecule that is bombesin or an analog thereof.

174. A stabilized radiopharmaceutical composition according to any one of claims 157 to 161, wherein the radionuclide is selected from the group consisting of ^{99m}Tc, ⁵¹Cr, ⁶⁷Ga, ⁶⁸Ga, ⁴⁷Sc, ¹⁶⁷Tm, ¹⁴¹Ce, ¹²³I, ¹²⁵I, ¹³¹I, ¹⁸F, ¹¹C, ¹⁵N, ¹¹¹In, ¹⁶⁸Yb, ¹⁷⁵Yb, ¹⁴⁰La, ⁹⁰Y, ⁸⁸Y, ^{86 Y,} ¹⁵³Sm, ¹⁶⁶Ho, ¹⁶⁵Dy, ¹⁶⁶Dy, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ²²⁵Ac, 211At, ¹⁰⁵Rh, ¹⁰⁹Pd, ^{117m}Sn, ¹⁴⁹Pm, ¹⁶¹Tb, ¹⁷⁷Lu, ¹⁹⁸Au and ¹⁹⁹Au and oxides or nitrides thereof.

175. A method for stabilizing a radiopharmaceutical composition either comprising combining a radionuclide with a chelator, so as to form a radiolabelled complex, and combining the complex with a stabilizer; or comprising simultaneously reacting a radionuclide with a chelator and with a stabilizer;
wherein the stabilizer is selected from the group consisting of: a stabilizer comprising a water-soluble organic compound containing selenium in the +2 oxidation state; a stabilizer composition comprising ascorbic acid or a pharmaceutically salt thereof, gentisic acid or a pharmaceutically salt thereof, human serum albumin, and benzyl alcohol; a stabilizer comprising a dithiocarbamate compound; and a stabilizer comprising a water soluble compound containing sulfur in the +2 oxidation state.

176. A method according to claim 175, wherein the stabilizer comprising a water-soluble compound containing selenium in the +2 oxidation state comprises selenomethionine, selenocysteine, or derivatives thereof.

177. A method according to claim 175, wherein the stabilizer composition comprising ascorbic acid or a pharmaceutically salt thereof, gentisic acid or a pharmaceutically salt thereof, human serum albumin, and benzyl alcohol further comprises selenomethionine, selenocysteine, methionine, cysteine or derivatives thereof.

178. A method according to claim 175 wherein the stabilizer comprising a dithiocarbamate compound comprises a compound of the formula: 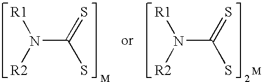
wherein R1 and R2 are each independently H; C₁-C₈ alkyl; —OR3, wherein R3 is C₁-C₈ alkyl; or benzyl, either unsubstituted or optionally substituted with water solubilizing groups; or
wherein R1, R2, and N combined together form 1-pyrrolidinyl-, piperidino-, morpholino-, 1-piperazinyl-; and
M is H⁺, Na⁺, K⁺, NH₄⁺, N-methylglucamine, or other pharmaceutically acceptable +1 ion, in the +1 oxidation state; or
M is Mg²⁺ or Ca²⁺, or other physiologically acceptable +2 metal ion, in the +2 oxidation state.

179. A method according to claim 175, wherein the the dithiocarbamate compound is selected from the group consisting of 1-pyrrolidine dithiocarbamic acid ammonium salt, sodium diethyldithiocarbamate trihydrate, sodium dimethyldithiocarbamate hydrate, and combinations thereof.

180. A method according to claim 175 wherein the stabilizer comprising a water soluble compound containing sulfur in the +2 oxidation state comprises cysteine or a derivative thereof, mercaptoethanol, dithiolthreitol, or pharmaceutically acceptable salts thereof.

181. A method according to claim 180 wherein the stabilizer comprising a water soluble compound containing sulfur in the +2 oxidation state comprises a cysteine derivative selected from the group consisting of cystamine dihydrochloride, cysteine hydrochloride monohydrate, cysteine ethyl ester hydrochloride, cysteine diethyl ester dihydrochloride, cysteine methyl ester hydrochloride, cysteine dimethyl ester dihydrochloride, cysteinesulfinic acid monohydrate, 5-thio-d-glucose, reduced 1-glutathione, and combinations thereof.

182. A kit for the preparation of a stabilized radiopharmaceutical composition comprising:
(a) a first reagent which comprises a diagnostic or therapeutic radionuclide, optionally complexed to a chelator; and
(b) a second reagent which comprises a stabilizer selected from the group consisting of: a stabilizer comprising a water-soluble organic compound containing selenium in the +2 oxidation state; a stabilizer composition comprising ascorbic acid or a pharmaceutically salt thereof, gentisic acid or a pharmaceutically salt thereof, human serum albumin, and benzyl alcohol; a stabilizer comprising a dithiocarbamate compound; and a stabilizer comprising a water soluble compound containing sulfur in the +2 oxidation state.

183. A kit according to claim 182 wherein the water-soluble compound containing selenium in the +2 oxidation state comprises selenomethionine, selenocysteine, or derivatives thereof.

184. A kit according to claim 182 wherein the stabilizer composition comprising ascorbic acid or a pharmaceutically salt thereof, gentisic acid or a pharmaceutically salt thereof, human serum albumin, and benzyl alcohol; further comprises selenomethionine, selenocysteine, methionine, cysteine or derivatives thereof.

185. A kit according to claim 182 wherein the stabilizer comprising a dithiocarbamate compound comprises a compound of the formula: 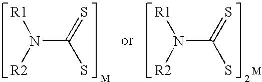
wherein R1 and R2 are each independently H; C₁-C₈ alkyl; —OR3, wherein R3 is C₁-C₈ alkyl; or benzyl, either unsubstituted or optionally substituted with water solubilizing groups; or wherein R1, R2, and N combined together form 1-pyrrolidinyl-, piperidino-, morpholino-, 1-piperazinyl-; and
M is H⁺, Na⁺, K⁺, NH₄⁺, N-methylglucamine, or other pharmaceutically acceptable +1 ion, in the +1 oxidation state; or
M is Mg²⁺ or Ca²⁺, or other physiologically acceptable +2 metal ion, in the +2 oxidation state.

186. A kit according to claim 182, wherein the the dithiocarbamate compound is selected from the group consisting of I -pyrrolidine dithiocarbamic acid ammonium salt, sodium diethyldithiocarbamate trihydrate, sodium dimethyldithiocarbamate hydrate, and combinations thereof.

187. A kit according to claim 182 wherein the stabilizer comprising a water soluble compound containing sulfur in the +2 oxidation state comprises cysteine or a derivative thereof, mercaptoethanol, dithiolthreitol, or pharmaceutically acceptable salts thereof.

188. A kit according to claim 187 wherein the stabilizer comprising a water soluble compound containing sulfur in the +2 oxidation state comprises a cysteine derivative selected from the group consisting of cystamine dihydrochloride, cysteine hydrochloride monohydrate, cysteine ethyl ester hydrochloride, cysteine diethyl ester dihydrochloride, cysteine methyl ester hydrochloride, cysteine dimethyl ester dihydrochloride, cysteinesulfinic acid monohydrate, 5-thio-d-glucose, reduced 1-glutathione, and combinations thereof.

189. A stabilized radiopharmaceutical composition comprising a compound A or B of formula: 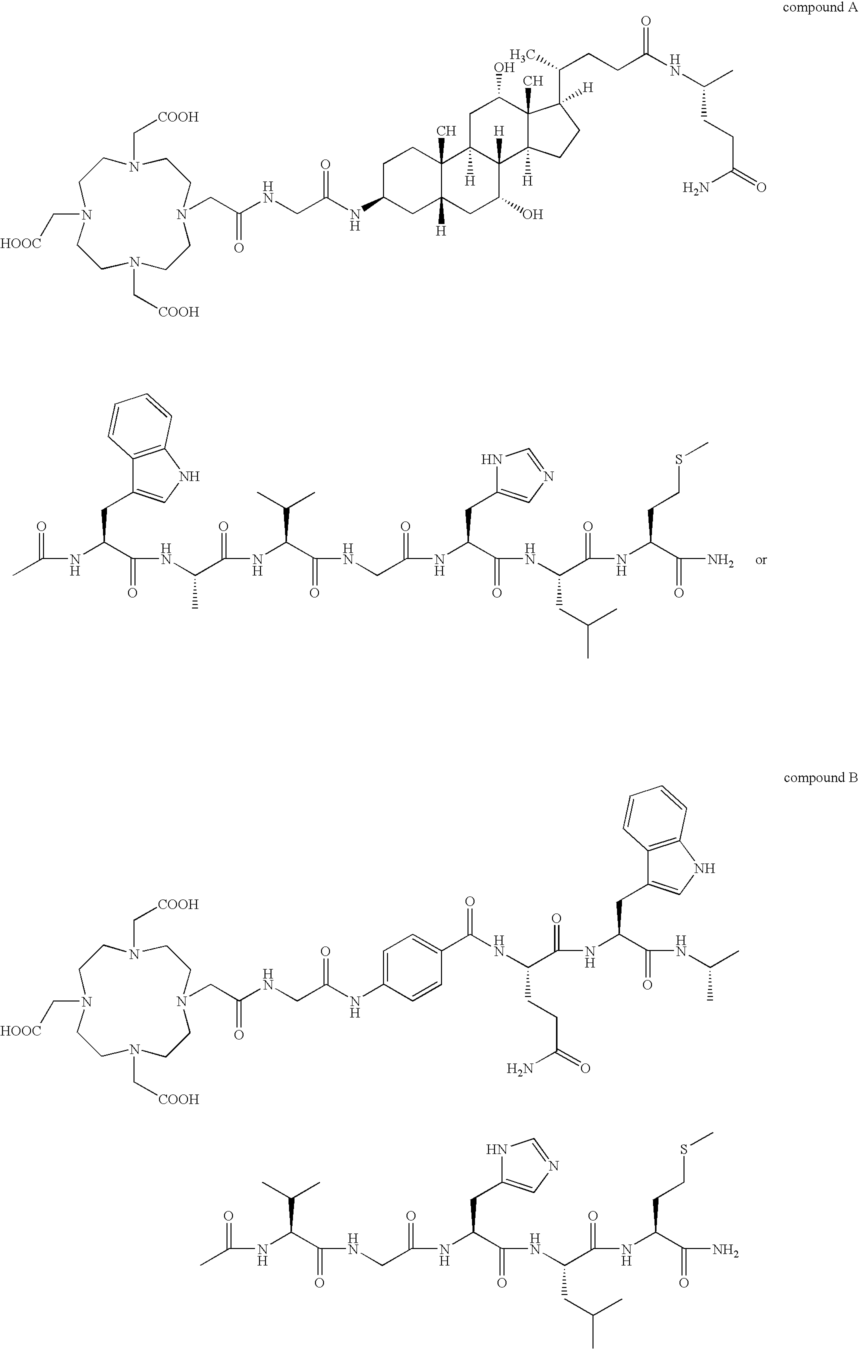
and a stabilizing composition comprising ascorbic acid, gentisic acid, human serum albumin, benzyl alcohol, and an amino acid selected from the group consisting of cysteine, methionine, or selenomethionine.

190. A kit for the preparation of a stabilized radiopharmaceutical composition comprising:
(a) a first reagent which comprises a compound of formula A or B, 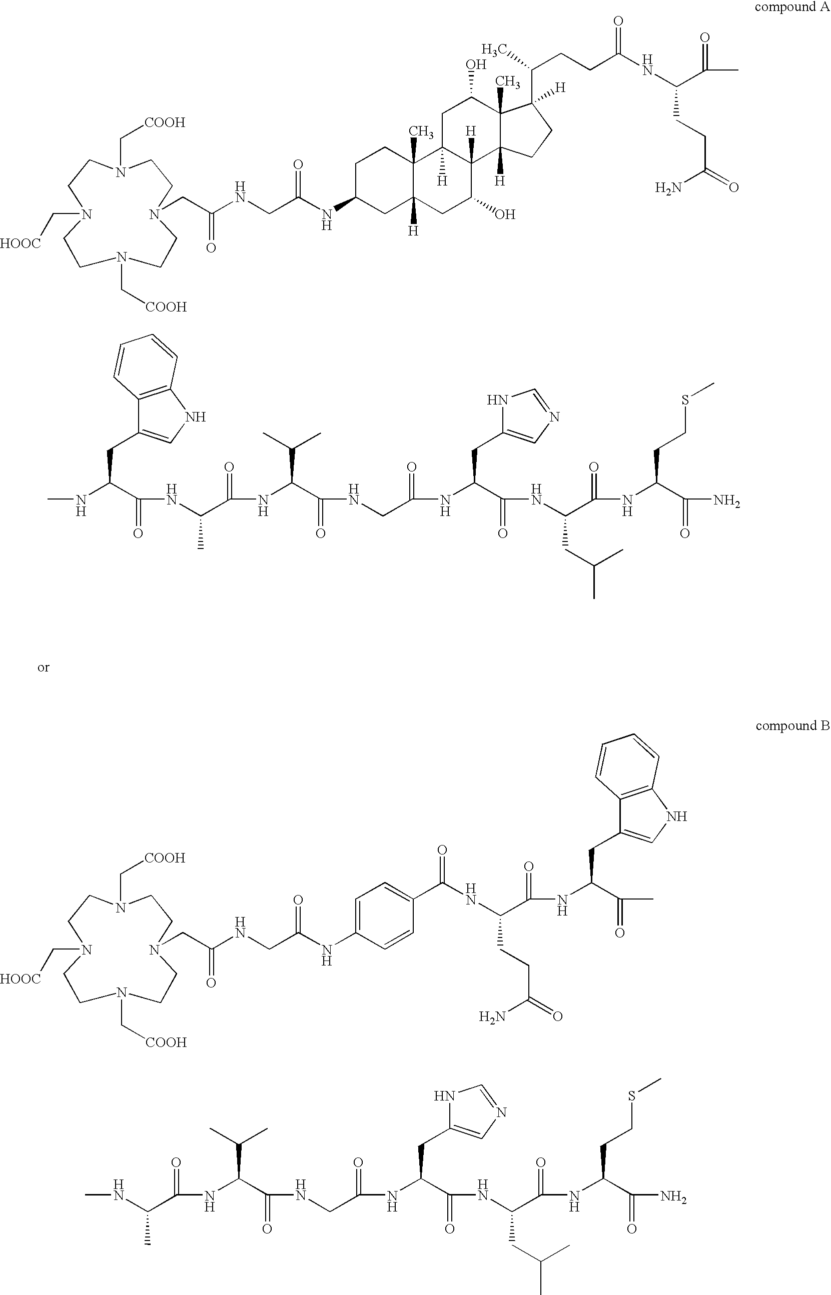
and a water-soluble organic compound containing selenium in the +2 oxidation state; and
(b) a second reagent which comprises ascorbic acid or a pharmaceutically salt thereof, sodium chloride, EDTA, and benzyl alcohol.

191. A kit according to claim 190, wherein the first reagent further comprises a radionuclide and wherein the compound containing selenium in the +2 oxidation state is selenomethionine.

192. A kit according to claim 191, wherein the radionuclide is selected from the group consisting of ¹⁷⁷Lu, ¹¹¹In, and ⁹⁰Y.

193. A method of increasing recovery of radioactivity from a reaction that produces a radiopharmaceutical composition either comprising:
adding benzyl alcohol to a reaction mixture that produces the radiopharmaceutical composition; or
reacting a radionuclide with a chelator, to form a radiolabeled chelate, and reacting the radiolabeled chelate with a stabilizer solution comprising benzyl alcohol.

194. A method according to claim 193, wherein the stabilizer solution further comprises ascorbic acid or a pharmaceutically acceptable salt thereof or EDTA.

195. A method of reducing one or more oxidized methionine residues in a radiopharmaceutical composition comprising reacting the radiopharmaceutical composition with cysteine, dithiolthreitol or mercaptoethanol.

196. A method according to claim 195, wherein the radiopharmaceutical composition comprises a compound having the formula of compound A or of compound B compound A

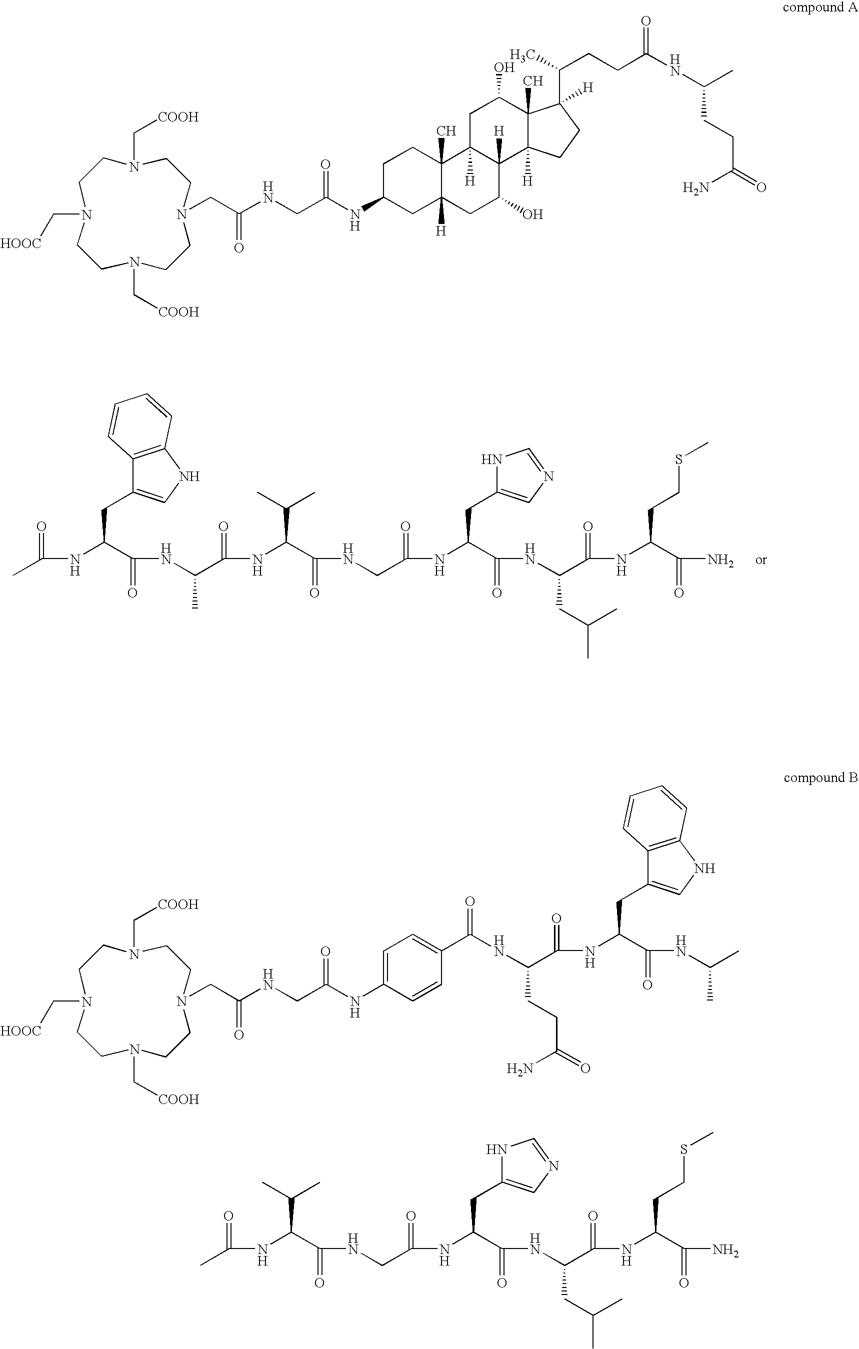

197. A method of reducing interference from metallic contaminants in a reaction mixture for the preparation of a radiopharmaceutical comprising reacting the mixture with a dithiocarbamate.

198. A method of improving yield of a desired radiopharmaceutical, comprising adding a dithiocarbamate to the reaction mixture that produces the radiopharmaceutical.

199. A method according to any one of claims 197 or 198, wherein the dithiocarbamate is 1-pyrrolidine dithiocarbamic acid ammonium salt (PDTC).
